(19) United States
(12) Patent Application Publication
(10) Pub. No.: US 2013/0029928 A1
(43) Pub. Date: Jan. 31, 2013

(54) COMPOSITIONS COMPRISING PERFLUOROOCTANOIC ACID

(71) Applicants: Clifford Roy Elcombe, Dundee (GB); Charles Roland Wolf, Dundee (GB); Anna Louise Westwood, Dundee (GB)

(72) Inventors: Clifford Roy Elcombe, Dundee (GB); Charles Roland Wolf, Dundee (GB); Anna Louise Westwood, Dundee (GB)

(21) Appl. No.: 13/579,774

(22) PCT Filed: Feb. 18, 2011
(86) PCT No.: PCT/GB2011/000232

§ 371 Date: Oct. 17, 2012

(30) Foreign Application Priority Data
Feb. 19, 2010 (GB) 1002861.1

(51) Int. Cl. A61K 31/20; A61K 31/704; A61K 31/7068; A61K 31/52; A61P 35/02; A61K 31/513; A61K 31/395; A61K 31/69; A61P 35/00; C07C 53/21; A61K 31/436

(52) U.S. Cl. 514/34; 554/226; 514/558; 514/49; 514/263.4; 514/291; 514/274; 514/183; 514/64

(57) ABSTRACT

There is provided compositions comprising perfluorooctanoic acid (PFOA) or a salt, derivative or variant thereof. There is also provided uses, methods therapeutic systems and combination therapies relating to PFOA.

The invention relates to compositions for treating cancer. In particular there is provided, doses, dosage regimes for the administration of Perfluorooctanoate (PFOA) and in particular, Ammonium Perfluorooctanoate (APFO) in the treatment of cancer.

Ammonium Perfluorooctanoate (APFO) has the molecular formula C₈F₁₅O₂.H₄N

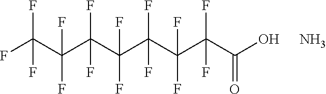

APFO is the ammonium salt of straight chain perfluorooctanoic acid (PFOA). Commercially available ammonium perfluorooctanoate (APFO) is a mixture of approximately 75% straight chain and 25% various branched isomers.

Preliminary experiments to evaluate mode of action were performed using this mixture (APFO). We have previously described (WO 2004/019927 WO 2002/66028) the use of perfluorinated carboxylic acids for the treatment of cancer.

Subsequently, the purified straight chain isomer was obtained, and the results obtained with APFO were verified with this isomer (CXR1002).

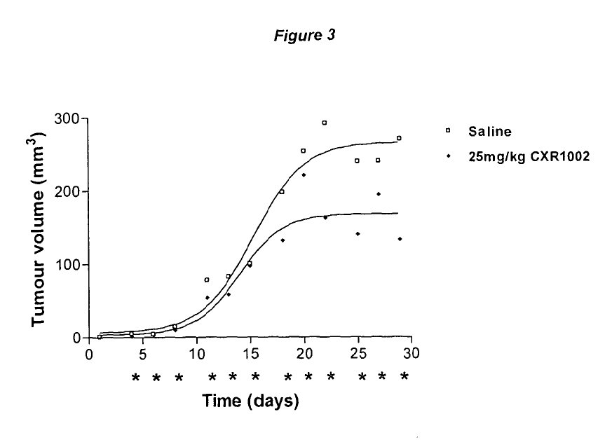

CXR1002 is a fatty acid mimetic in that it interacts with fatty acid homeostasis and/or a fatty acid mediated pathway. Both CXR1002 and APFO isomers and also perfluoroalkyls of different chain lengths possess these properties. This has been demonstrated in Vanden Heuvel (1996) where it was shown that different nuclear hormone receptors were activated by PFOA and how this compared to natural fatty acid activation of the same receptors. Wolf (2008) showed a dose response of various chain length perfluoroalkyls against PPAR alpha (FIG. 3 of Wolf (2008)) in a transiently transfected COS-1 cell model to compare the C4 to C9 chain lengths.

It has now been shown that APFO and the CXR1002 isomer has additional mechanisms of action accounting for some of its anti-tumour effects.

APFO has been shown to cause Endoplasmic Reticulum (ER) stress (see Example 8). Endoplasmic reticulum stress induction has been shown to have an anti-tumour effect, including in pancreatic cancer, myeloma and thyroid cancer. For example, sorafenib, bortezomib and Hsp90 cause cell death by induction of ER stress pathways and bortezomib is used clinically to treat multiple myeloma and mantle cell lymphoma. Review articles discussing the association with ER stress and Cancer are Healy (2009), Strasser (2008) and Moenner (2007).

APFO has also been shown to have activity against PIM kinases (see Example 9). PIM kinases are cytoplasmic serine/threonine kinases that are known to be involved in regulation of apoptosis and cellular metabolism. Certain PIM kinases have been shown to be upregulated in cancers and as such their inhibition represents a mechanism of action by which CXR1002 can have an anti-tumour effect in conditions such as leukaemia, lymphoma, prostate cancer, colon cancer and pancreatic cancer. The below studies have shown this link:
Liver cancer: Gong (2009), Fujii (2005) and Wu (2010) have shown PIM-2 to promote tumorigenesis and PIM-3 to accelerate hepatocellular carcinoma development when induced by hepatocarcinogen.
Gastric cancer: Zhen (2008) and Warnecke-Eberz (2009) have shown overexpression of PIM-1 in gastric glands to be associated with lymph node metastases.
Head and neck cancer: Beier (2007) has shown PIM-1 overexpression in head and neck squamous cell carcinomas.
Colon cancer: Popivanova (2007) has shown PIM-3 to be aberrantly expressed in human colon cancer cells but not normal colon mucosa.
Pancreatic cancer: Li (2006), Chen (2009) and Reiser-Erkan (2008) have shown PIM-3 expression occurs in human pancreatic cancer but not normal cells and PIM-1 blockage using siRNA resensitises pancreatic cancer cells to apoptosis and PIM-1 levels correlate to clinicopathological parameters in pancreatic cancer.
Leukaemia/lymphoma: Adam (2006), Hammerman (2005), Cohen (2004), Hogan (2008), Lin (2010), Kim (2005), Chen (2008) and Brault (2010) have shown PIM-2 expression is increased in leukaemia/lymphoma, expression of PIM-1 and PIM-2 is dependent on Abl kinase activity and PIM-1 mediates homing and migration of malignant haematopoietic cells.
Oral cancer: Chiang (2006) and Choi (2010) have shown PIM-1 expression to be high in squamous cell carcinoma.
Prostrate cancer: Chen (2005), Mumenthaler (2009), He (2007), Xu (2005), Dai (2005) and Roh (2008) have shown PIM-1 overexpression in prostatic carcinoma.
Breast cancer: Roh (2008) has shown PIM-1 overexpression to convert mammary epithelia cells to become tumourgenic.
Adipocyte tumours: Nga (2010) has shown benign and malignant adipocytic tumours to have strong PIM-1 expression.

PIM kinases are constitutively active and their activity as shown above and in Amaravadi (2005) and Shah (2008) supports in vitro and in vivo human cell growth and survival.

APFO is a perfluorinated carboxylic acid that exerts its anti-tumour effects via multiple mechanisms of action. Previously it had been know that APFO acts by one or more peroxisome proliferator activated receptor (PPAR)-mediated mechanisms. PPARs are members of the nuclear hormone receptor family of transcription factors. They modulate DNA transcription by binding to specific peroxisome proliferator-response elements (PPREs) on target genes.

CXR1002 is a white, odourless solid that is freely soluble in water. CXR1002 and its family of compounds are extremely stable.

The investigational medicinal product being made in the clinical trials described in the examples consists of Size 1 white opaque gelatin capsules containing the active substance, CXR 1002. There is no bulking agent. One strength of capsule has been manufactured with a target strength of 50 mg of CXR1002 per capsule.

Laboratory studies have indicated that CXR1002 can interact with cells in a number of different ways which could be associated with its pharmacological effectiveness as an anti-tumour agent. For example, CXR1002 is an agonist of PPARs and also induces ER-stress in tumour cells. CXR1002 has also been shown to have a range of biological effects probably related to its surfactant properties, including; alteration of cell membrane potential and cytostolic pH (Kleszczynski (2009)); induction of oxidative stress (Fernandez (2008)) that was closely linked to cell cycle arrest; dissipation of mitochondrial membrane potential (Hu (2009)) and dysregulation of gap-junctional intercellular communication (GJIC) and activation of extracellular receptor kinase (ERK) (Upham (2009)). CXR1002 is cytotoxic to tumour cells with an IC₅₀ ranging upwards from 273 μM.

The data presented demonstrate that CXR1002 has anti-tumour activity both in vitro and in xenograft models. The mechanism of action, involving agonism of PPARs α and γ in association with neutral or inhibitory action on PPARδ, is distinct from those of currently available chemotherapeutic agents. In addition CXR1002 induces ER-stress in some cancer cell lines; this may be an effect that is related to its effects on PPARs. Furthermore CXR1002 is an inhibitor of the PIM kinase family of serine/threonine kinases. CXR1002 could provide anticancer activity against a range of tumour types. Humans have already received environmental exposure to CXR1002 and workers involved in the manufacture of APFO have been recorded as having serum concentrations as high as 275 μM without reported adverse effects. Furthermore, patients in the ongoing CXR1002-001 study have exposure in the 200 μM to 800 μM range after a few weeks of dosing with CXR1002. This level of exposure to cells in vitro or to a xenografted tumour would be expected to have a biological effect.

As of February 2011, 43 patients with advanced cancers from one Phase I study have received CXR1002. CXR1002 is not metabolised and dosing is accumulative. It is presumed that CXR1002 will eventually reach a steady state level after a number of doses, in an analogous way to its accumulative exposure in monkeys. The lack of metabolism of CXR1002 provides an advantage over other chemotherapeutic agents such that inter-patient variability in exposure is low as metabolism of the active ingredient at different rates in different patients is not an issue for CXR1002.

Significant occupational exposure to PFOA and its salts, including APFO, has occurred over many years and APFO has been found in the blood of workers exposed in the workplace. The dogma derived from studies such as these is that CXR1002 has a long serum half-life in humans (range=109 to 1308 days). Data from the CXR1002-001 clinical trial, demonstrate that after a single dose of CXR1002, the plasma level of the drug is constant over the 6 week sampling period, indicating that the half life is >6 weeks.

However, patients in the phase I study receiving >100 mg weekly dose have higher exposure after 6 weeks of dosing than the maximal values recorded in occupationally exposed workers.

A large database of experimental studies on the potential health hazards of APFO is available, as are recent toxicology reviews (USEPA (2005)), (Kennedy (2004)). In addition to toxicology studies in laboratory animals, the potential association of APFO exposure with health effects in fluorochemical production workers has been studied since 1976 through medical monitoring and epidemiological investigations (Ubel (1980)), (Olsen (1998)), (Olsen (2000)).

The majority of studies reported in the literature have used APFO itself, although some studies using other salts have also been described. The biological effects of APFO are thought to be due to its dissociation to form perfluorooctanoate (PFOA), the anionic form of perfluorooctanoic acid. Perfluorooctanoic acid and its salts are soluble in water and readily dissociates to the carboxylate anion, perfluorooctanoate (PFOA) (Kennedy (2004)).

The consensus is that, since the active constituent of each of these compounds is the perfluorooctanoate anion, these studies are directly comparable. An extensive toxicology and occupational health database already exists for this compound. Several studies of relevance have been commissioned by commercial companies but the reports are not in the public domain. However, the field has been thoroughly reviewed by Kennedy et al. (2004) and the USEPA (2005). In addition, key studies have been published in the scientific literature or are available through the USEPA public docket.

Most commercial studies on APFO/PFOA have used a commercial material e.g. FC-143 FLUORAD, which comprises 93-97% APFO and the remaining consisting of a mixture of Ammonium perfluoropentanoate, Ammonium perfluoroheptanoate and Ammonium perfluorohexanaote.

Unlike most other anti-tumour agents, PFOA is efficiently absorbed following oral exposure. It is not metabolised and is eliminated intact. PFOA exhibits only moderate acute oral toxicity. Signs and symptoms of toxicity include body weight loss, liver weight increase and liver effects as demonstrated by increased serum transaminase activity and diffuse hepatocellular hypertrophy accompanied, at higher doses, by acidophilic degeneration and/or necrosis of the liver. PFOA exhibits no teratogenic or foetotoxic effects in rats at doses below those causing maternal toxicity and there is no evidence of any adverse effects on reproductive success in a two-generation reproduction study. Two year cancer bioassays in rats resulted in increased incidence of benign tumours (adenomas) of the liver, pancreas (acinar cell) and testes (Leydig cell) at 300 ppm in the diet, but not at 30 ppm. A battery of tests for genotoxicity demonstrated that PFOA does not cause either point mutations or chromosomal aberrations.

None of the toxicology studies give any indication of changes in cardiovascular, central nervous system, respiratory or renal function induced by PFOA. Studies in rats have revealed no clinical signs that suggested adverse pharmacological effects. Furthermore, there was no evidence of such effects in a 26-week toxicity study in male cynomolgus monkeys.

Although no specific studies have been carried out in humans on the potential unwanted pharmacological effects of PFOA, there are no significant toxicities reported in workers with significant occupational exposure.

PFOA is well absorbed following oral exposure. After a single oral dose of ¹⁴C-PFOA (11 mg/kg) to male rats at least 93% of total radioactivity was absorbed at 24 hrs (58). Following a single gavage administration to rats (25 mg/kg), peak blood levels were attained 1-2 hours after dosing (Kennedy (2004)). There was a clear sex difference in clearance. Blood levels in female rats showed >95% clearance 24 hrs after dosing, while blood levels in males remained relatively high throughout this period. The sex difference in clearance was even more marked 1 week after treatment, when blood levels in males remained relatively high and those in females had declined to very low levels.

Importantly, PFOA does not appear to accumulate in blood of female rats, since the blood profile of an oral dose of 25 mg/kg following 10 previous similar doses was quite similar to that observed after a single oral dose (Kennedy (2004)).

The amounts of PFOA deposited in the tissues of different species are inversely related to the species-specific rate of urinary excretion. In species which excrete PFOA slowly, the compound distributes primarily to the liver, plasma and the kidney and to a lesser extent other tissues of the body, including testis and ovary. For example, following 28 days gavage administration to male rats the major sites of deposition were the serum, liver and kidney. Little transfer to the brain occurs in adults. In female rats, the pattern of tissue deposition is dose-dependent. At 3 mg/kg more PFOA is deposited in the liver than the kidney whereas this is reversed at higher doses, suggesting the existence of a saturable renal excretory mechanism in the (female) rat (Kennedy (2004)).

There is no evidence that APFO is metabolised in mammals once dissociated to form perfluorooctanoate. However, analysis of five major drug metabolising cytochrome P450 (CYP) isozymes (CYP1A2, CYP2C9, CYP2C19, CYP2D6 and CYP3A4) indicated that CXR1002 is an inhibitor of CYP2C9, having an IC₅₀ of 0.76 μM under the conditions used (unpublished data). Similar results were obtained using APFO, which had an IC₅₀ of 0.78 μM towards CYP2C9.

The main factor determining the elimination rate of PFOA in different species is the rate of urinary excretion. In female rats, the extent of biliary excretion is <1.0% (Vanden (1991)).

The human renal clearance of PFOA has been evaluated in Japanese volunteers (Hasada (2005)). There were no significant differences in the renal clearance of PFOA with regard to sex, age group, medication, and medical or residential history.

To date, studies of PFOA have primarily related to the effects of the compound as a contaminant and occupational exposure in humans. Little is known regarding its safe and effective use as a therapeutic agent. Safe and efficacious dosages and therapeutic administration regimes have now been identified, specifically in relation to the treatment of cancer.

Furthermore, combinations of PFOA and other chemotherapeutic agents that are unexpectedly advantageous have also been identified as part of the invention.

APFO, and in particular the CXR1002 isomer has a large number of beneficial properties in comparison to existing chemotherapeutic agents. For example, CXR1002 is highly water soluble and as such is highly bioavailable. The high bioavailability is partially explained by CXR1002 possessing a long half life (shown to be greater than 6 weeks of half life in the clinical trials discussed in the examples). CXR1002 is now known not to be a substrate for human metabolism and as such dose and plasma concentration are closely linked and importantly variation between individuals is minimal (as there is no metabolism of CXR1002 there is no variability between individuals in metabolism). The slow clearance of CXR1002 means that a missed dose can be easily compensated for at a later date without an extensive loss of exposure to CXR1002. Due to the low variability of CXR1002 metabolism and clearance between individuals, dose strength and dose frequency required to achieve a desired plasma concentration is readily calculable by a skilled person because circulating plasma concentration can be reliably predicted from each dose taken.

CXR1002 has been shown in the clinical trials described in the examples to be orally bioavailable and this allows for simpler administration than current chemotherapeutic treatments (which are often given by intravenous administration), even to the point of allowing CXR1002 to be taken by patients outside of a hospital setting. In addition the CXR1002 capsule formulation has at least a 57 month shelf life that is commercially useful. The clinical trial work being conducted on CXR1002 has shown that CXR1002 is relatively non-toxic (at the doses examined to date CXR1002 does not cause toxicity commonly associated with anti-cancer drugs (no myelosuppresion, no anaemia, no transfusion requirement, no hair loss, mild or no effect on digestive system (individual variability apparent), no mouth ulcers, no skin problems, no lung effects, no heart effects, no neuropathy or nerve changes).

Although there is some reported nausea and vomiting with CXR1002, study subjects are not receiving concomitant anti-emetics, and these adverse events are of short duration.

Although CXR1002 causes liver enzyme changes in many toxicological test species (such as rats), the frequency of this in study subjects is low, with the predominant side effects being relatively mild including lethargy and mild gastrointestinal disturbance, nausea/vomiting and diarrhoea). The low toxicity of CXR1002 is supported by evaluation of pharmacodynamic markers in the clinical trials as discussed in the examples, which has shown there to be no significant changes.

The low toxicity profile and lack of metabolism allow CXR1002 to be used in combination with other therapeutic regimes with significant side-effects including cytotoxic chemotherapeutics and radiotherapy. Unlike other chemotherapeutics, CXR1002 can be used at the same time or prior to surgery with no wash out period required as CXR1002 would not exhibit the same side-effects as other chemotherapeutics on wound healing and immune response (due to the low toxicity of CXR1002).

Hence CXR1002 has been shown to possess significant advantages over other chemotherapeutics, these advantages allowing the specific compositions, dosage regimes and combination therapies to be identified and optimized as herein described.

In a first aspect of the invention there is provided a composition comprising between 10 mg and 2000 mg of an active ingredient per dosage unit, wherein the active ingredient is perfluorooctanoic acid (PFOA) or a derivative, salt or variant thereof.

By dosage unit we mean the unit of medicament administered to a patient at one time. For example, the dosage unit, or single dose may be administered by a single capsule/tablet, single injection, or single intravenous infusion, a single subcutaneous injection, or by a single procedure using other routes of administration, as discussed below. Alternatively, the single dose may be administered to the patient by two or more capsules/tablets or injections given simultaneously or sequentially to deliver the entire dose to the patient in the continuous, single and defined treatment period; by two or more intravenous infusions given simultaneously or sequentially to deliver the entire dose to the patient in the continuous, single and defined treatment; or by multiple procedures using other routes of administration as discussed below.

Alternatively, the single dose to be administered to the patient can be delivered by a combination of routes to deliver the entire dose to the patient in the continuous, single and defined treatment.

The dosage unit may then be repeated at intervals of time such as a few hours, days, weeks, or months later.

Dosage units can be administered to patients in such a way that the patient receives a loading dose followed by one or more maintenance doses. For example the loading dose may be a high dose in order to quickly reach a desired plasma concentration and then subsequent maintenance doses are a lower dose than the loading dose in order to maintain the required plasma concentration.

By active ingredient we mean the molecule having the desired effect. In this case of this invention we primarily mean PFOA and derivatives, salts or variants thereof.

By variants and derivatives we mean any molecules of substantially identical chemical structure but including minor modifications that do not alter activity but may offer improved or alternative properties for formulation, such as formation into a salt.

In human therapy, the PFOA containing composition, and medicaments of the invention can be administered alone but will generally be administered in admixture with a suitable pharmaceutical excipient, diluent or carrier selected with regard to the intended route of administration and standard pharmaceutical practice.

For example, the PFOA containing composition, and medicaments of the invention can be administered orally, buccally or sublingually in the form of tablets, capsules, ovules, elixirs, solutions or suspensions, which may contain flavouring or colouring agents, for immediate-, delayed- or controlled-release applications. The PFOA containing composition, and medicaments of the invention may also be administered via intracavernosal injection.

Such tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elixirs, the PFOA containing composition, medicaments and pharmaceutical compositions of the invention may be combined with various sweetening or flavouring agents, colouring matter or dyes, with emulsifying and/or suspending agents and with diluents such as water, ethanol, propylene glycol and glycerin, and combinations thereof.

The PFOA containing composition, and medicaments of the invention can also be administered parenterally, for example, intravenously, intra-arterially, intraperitoneally, intra-thecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Medicaments and pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The medicaments and pharmaceutical compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

The PFOA containing composition, and medicaments of the invention can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active agent, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a PFOA containing composition, of the invention and a suitable powder base such as lactose or starch.

Aerosol or dry powder formulations are preferably arranged so that each metered dose or “puff” contains an effective amount of an agent or polynucleotide of the invention for delivery to the patient. It will be appreciated that the overall daily dose with an aerosol will vary from patient to patient, and may be administered in a single dose or, more usually, in divided doses throughout the day.

Alternatively, the PFOA containing composition, and medicaments of the invention can be administered in the form of a suppository or pessary, or they may be applied topically in the form of a lotion, solution, cream, gel, ointment or dusting powder. The PFOA containing composition, and medicaments of the invention may also be transdermally administered, for example, by the use of a skin patch. They may also be administered by the ocular route, particularly for treating diseases of the eye.

For ophthalmic use, the PFOA containing composition, and medicaments of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the PFOA containing composition, and medicaments of the invention can be formulated as a suitable ointment containing the active agent suspended or dissolved in, for example, a mixture with one or more of the following: mineral oil, liquid petrolatum, white petrolatum, propylene glycol, polyoxyethylene polyoxypropylene agent, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Generally, in humans, oral or parenteral administration of the PFOA containing composition, medicaments and pharmaceutical compositions of the invention is the preferred route, being the most convenient.

For veterinary use, the PFOA containing composition, and medicaments of the invention are administered as a suitably acceptable formulation in accordance with normal veterinary practice and the veterinary surgeon will determine the dosing regimen and route of administration which will be most appropriate for a particular animal.

The PFOA containing composition, as defined herein may be formulated as described in the accompanying Examples.

Preferably the PFOA is ammonium perfluorooctanoic acid (APFO), the ammonium salt.

The composition may comprise any effective amount of active ingredient, this may be between 10 mg and 2000 mg of active ingredient per dosage unit, and preferably is between 50 mg and 1000 mg. Advantageously it is 1000 mg. Conveniently, the dosage unit contains an amount of active ingredient per dosage unit selected from 10 mg, 20 mg, 25 mg, 50 mg, 100 mg, 200 mg, 300 mg, 400 mg, 450 mg, 600 mg, 750 mg, 950 mg, 1000 mg and 1200 mg.

Alternatively, the composition may comprise between 10-50 mg, 10-75 mg, 10-100 mg, 10-200 mg, 10-300 mg, 10-400 mg, 10-600 mg, 10-750 mg, 10-950 mg, 10-1000 mg, 10-1200 mg, 50-75 mg, 50-100 mg, 50-200 mg, 50-300 mg, 50-450 mg, 50-600 mg, 50-750 mg, 50-950 mg, 50-1000 mg, 50-1200 mg, 75-100 mg, 75-200 mg, 75-300 mg, 75-450 mg, 75-600 mg, 75-750 mg, 75-950 mg, 75-1000 mg, 75-1200 mg, 100-200 mg, 100-300 mg, 100-450 mg, 100-600 mg, 100-750 mg, 100-950 mg, 100-1000 mg, 100-1200 mg, 200-300 mg, 200-450 mg, 200-600 mg, 200-750 mg, 200-950 mg, 200-1000 mg, 200-1200 mg, 300-450 mg, 300-600 mg, 300-750 mg, 300-950 mg, 300-1000 mg, 300-1200 mg, 400-600 mg, 400-750 mg, 400-950 mg, 400-1000 mg, 400-1200 mg, 450-600 mg, 450-750 mg, 450-950 mg, 450-1000 mg, 450-1200 mg, 600-750 mg, 600-950 mg, 600-1000 mg, 600-1200 mg, 700-950 mg, 700-1000 mg, 700-1200 mg, 950-1000 mg, 950-1200 mg and 1000-1200 mg

Preferably there is 400-600 mg of active ingredient. More preferably there is 400-1200 mg of active ingredient. Most preferably there is 1000 mg of active ingredient.

Conveniently, the composition is pharmaceutically acceptable, and may optionally contain a pharmaceutically acceptable excipient, diluent, carrier or filler.

In a second aspect of the invention there is provided a composition as defined in the first aspect of the invention for use as a medicine.

In a third aspect of the invention there is provided a composition as defined in the first aspect of the invention for use in the treatment of cancer.

By “treatment” we include the meanings that tumour size is reduced and/or further tumour growth is retarded and/or prevented and/or the tumour is killed. We also include the reduction of other symptoms associated with the cancer being treated such as (but not limited to) a reduction in pain, cachexia and metastasis. The treatment may incorporate multiple aspects including chemotherapy, surgery and radiotherapy. The composition of the invention may be used on its own as a chemotherapeutic or with any other treatment for cancer, including before, during and after any other treatment type.

By ‘treatment’ we include both therapeutic and prophylactic treatment of a subject/patient. The term ‘prophylactic’ is used to encompass the use of composition described herein which either prevents or reduces the likelihood of the occurrence or development of cancer in a patient or subject.

A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce or prevent a clinically significant deficit in the activity, function and response of the host. Alternatively, a therapeutically effective amount is sufficient to cause an improvement in a clinically significant condition in a host.

In a fourth aspect of the invention there is provided a use of a composition as defined in the first aspect of the invention in the manufacture of a medicament for the treatment of cancer.

In a fifth aspect of the invention there is provided a method of treating cancer comprising administering an effective amount of a composition as defined in the first aspect of the invention. Preferably the effective amount is between 10 and 2000 mg per dose, preferably between 50 and 600 mg per dose, and more preferably between 50 and 1200 mg per dose. Alternatively the effective amount is between 1 and 20 mg/kg, preferably between 1 and 7 mg/kg.

As is appreciated by those skilled in the art, the precise amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art.

In a particularly preferred embodiment, the amount of the active ingredient administered to a patient is approximately between: 0.02 mg/kg to 0.10 mg/kg; or 0.10 mg to 0.20 mg/kg; or 0.20 mg to 0.30 mg/kg; or 0.30 mg to 0.40 mg/kg; or 0.40 mg to 0.50 mg/kg; or 0.50 mg to 0.60 mg/kg; or 0.60 mg to 0.70 mg/kg; or 0.70 mg to 0.80 mg/kg; or 0.80 mg to 0.90 mg/kg; or 0.90 mg to 1.00 mg/kg; or 1.00 mg to 1.10 mg/kg; or 1.10 mg to 1.20 mg/kg; or 1.20 mg to 1.30 mg/kg; or 1.30 mg to 1.40 mg/kg; or 1.40 mg to 1.50 mg/kg; or 1.50 mg to 1.60 mg/kg; or 1.60 mg to 1.70 mg/kg; or 1.70 mg to 1.80 mg/kg; or 1.80 mg to 1.90 mg/kg; or 1.90 mg to 2.00 mg/kg; or 2.00 mg/kg to 2.10 mg/kg; or 2.10 mg to 2.20 mg/kg; or 2.20 mg to 2.30 mg/kg; or 2.30 mg to 2.40 mg/kg; or 2.40 mg to 2.50 mg/kg; or 2.50 mg to 2.60 mg/kg; or 2.60 mg to 2.70 mg/kg; or 2.70 mg to 2.80 mg/kg; or 2.80 mg to 2.90 mg/kg; or 2.90 mg to 3.00 mg/kg; or 3.00 mg to 3.10 mg/kg; or 3.10 mg to 3.20 mg/kg; or 3.20 mg to 3.30 mg/kg; or 3.30 mg to 3.40 mg/kg; or 3.40 mg to 3.50 mg/kg; or 3.50 mg to 3.60 mg/kg; or 3.60 mg to 3.70 mg/kg; or 3.70 mg to 3.80 mg/kg; or 3.80 mg to 3.90 mg/kg; or 3.90 mg to 4.00 mg/kg; or 4.00 mg to 4.10 mg/kg; or 4.10 mg to 4.20 mg/kg; or 4.20 mg to 4.30 mg/kg; or 4.30 mg to 4.40 mg/kg; or 4.40 mg to 4.50 mg/kg; or 4.50 mg to 4.60 mg/kg; or 4.60 mg to 4.70 mg/kg; or 4.70 mg to 4.80 mg/kg; or 4.80 mg to 4.90 mg/kg; or 4.90 mg to 5.00 mg/kg; or 5.00 mg/kg to 6.00 mg/kg; or 6.00 mg to 7.00 mg/kg; or 7.00 mg to 8.00 mg/kg; or 8.00 mg to 9.00 mg/kg; or 9.00 mg to 10.00 mg/kg; or 10.00 mg to 11.00 mg/kg; or 11.00 mg to 12.00 mg/kg; or 12.00 mg to 13.00 mg/kg; or 13.00 mg to 14.00 mg/kg; or 14.00 mg to 15.00 mg/kg; or 15.00 mg to 16.00 mg/kg; or 16.00 mg to 17.00 mg/kg; or 17.00 mg to 18.00 mg/kg; or 18.00 mg to 19.00 mg/kg; or 19.00 mg to 20.00 mg/kg.

A composition, use or method of any of the third to fifth aspects wherein the treatment comprises the step of administering to a patient in need thereof an effective amount of the composition, in a single dosage at a frequency of once or twice per week (weekly or semi-weekly). Conveniently, the single dosage is administered at a frequency of less than once per week, preferably fortnightly or once per six weeks or less.

The dosage may be administered as a higher loading dose followed by one or more lower maintenance doses.

In a sixth aspect of the invention there is provided a therapeutic system for the treatment of cancer comprising administration of a composition as defined in the first aspect in a single dosage of between 10 mg and 2000 mg at a frequency of once per week or less.

By therapeutic system we mean a system of administering compositions to a patient in an effective manner to treat a specific disease. The system may be characterised by the dosages to be administered, the intervals between dosages and the methods of administration, or combinations thereof. The system may also be interchangeably known as a dosage regime.

Preferably, the dosage is between 200 mg and 1200 mg. Conveniently, the dosage is selected from 10 mg, 50 mg, 100 mg, 200 mg, 300 mg, 450 mg, 600 mg, 750 mg, 950 mg, 1000 mg and 1200 mg.

Alternatively, the dosage is selected from 1 mg/kg to 7 mg/kg.

Preferably, the dosage frequency is once per six weeks or less.

In the third to sixth aspects of the invention, the cancer may be selected from pancreatic cancer, ovarian cancer, breast cancer, prostate cancer, liver cancer, chondrosarcoma, lung cancer, head and neck cancer, colon cancer, sarcoma, leukaemia, lymphoma, kidney cancer, thyroid cancer and brain cancers such as glioblastoma.

In a seventh aspect of the invention there is provided a composition comprising perfluorooctanoic acid (PFOA) or a salt, derivative or variant thereof; and a further chemotherapeutic agent. Alternatively, there is provided a composition comprising an active ingredient as defined in the first aspect and a further chemotherapeutic agent.

Preferably, the further chemotherapeutic is selected from Doxorubicin, Gemcitabine, Roscovitine, Rapamycin, 5-FU, PARP inhibitors, kinase inhibitors including PIM kinase inhibitors and MAP kinase inhibitors, Hsp90 inhibitors (including Geldanamycin), proteasome inhibitors (including Bortezomib) and HDAC inhibitors (including SAHA); and prodrugs thereof.

Preferably, the further chemotherapeutic is present in an individually effective dose.

By individually effective dose we mean the dose at which the further chemotherapeutic is known to be effective when administered on its own.

Alternatively, the further chemotherapeutic is present in a lower than individually effective dose.

By lower than individually effective dose we mean a dose which is lower than that which is known to be the effective dose when the further chemotherapeutic is administered on its own. In other words, a lower dose than normal is administered because the combination provides a synergistic effect. This has the effect of reducing the administration of chemotherapeutics with unpleasant or dangerous side effects.

In an eighth aspect of the invention there is provided a composition as defined in the seventh aspect for use as a medicine.

In a ninth aspect there is provided a composition as defined in the seventh aspect for use in the treatment of cancer.

In a tenth aspect there is provided a use of a composition as defined in the seventh aspect in the manufacture of a medicament for the treatment of cancer.

In an eleventh aspect there is provided a method of treating cancer comprising administering an effective amount of a composition as defined in the seventh aspect.

In a twelfth aspect there is provided a therapeutic system for the treatment of cancer comprising a combination of component (i) a composition as defined in the first aspect; and (ii) a further chemotherapeutic agent, the components (i) and (ii) being provided for the use in the treatment of cancer and wherein components (i) and (ii) are administered in combination with one another.

By “in combination with one another” regarding the PFOA and chemotherapeutic agent treatments we include the meaning not only that the PFOA and chemotherapeutic agents are administered simultaneously, but also that they are administered separately and sequentially.

In one embodiment, administration of component (i) precedes administration of component (ii). In an alternative embodiment, administration of component (ii) precedes administration of component (i). In a further alternative embodiment, administration of component (i) occurs at the same time as administration of component (ii).

It is envisaged that the components may be administered in any order depending on individual circumstances including, need, drug availability, administration routes used. Preferably the PFOA and chemotherapeutic agents are administered between 0 and 24 hours apart with either the PFOA or the chemotherapeutic being administered first.

Preferably, the further chemotherapeutic of the therapeutic system is selected from Doxorubicin, Gemcitabine, Roscovitine, Rapamycin, 5-FU, PARP inhibitors, kinase inhibitors including PIM kinase inhibitors and MAP kinase inhibitors, Hsp90 inhibitors (including Geldanamycin), proteasome inhibitors (including Bortezomib) and HDAC inhibitors (including SAHA); and prodrugs thereof.

In particular chemotherapeutics that enhance or complement the mechanisms of action of the composition of the invention (CXR1002) are preferred e.g. Hsp90 inhibitors, proteasome inhibitors and HDAC inhibitors.

Hsp90 inhibitors, including geldanamycin, target the chaperone Hsp90 and promote ubiquitin-dependent proteasomal degradation of proteins, leading to ER stress. Bortezomib, a proteasome inhibitor, also promotes the accumulation of aggregated, ubiquitinated proteins in the ER and therefore also cause ER stress. HDAC inhibitors have been shown to act synergistically with bortezomib, indicating that they may be id useful together with agents that induce ER stress (such as CXR1002). PIM kinase inhibition can restore sensitivity to FLT3 and BCR/ABL mutations that confer resistance to tyrosine kinase inhibitors.

In the ninth to twelfth aspects, the cancer may be selected from pancreatic cancer, ovarian cancer, breast cancer, prostate cancer, liver cancer, chondrosarcoma, lung cancer, head and neck cancer, colon cancer, sarcoma, leukaemia, lymphoma, kidney cancer, thyroid cancer and brain cancers such as glioblastoma.

In one embodiment when the cancer is pancreatic cancer, the further chemotherapeutic is selected from Doxorubicin, Gemcitabine, Geldanamycin and Roscovitine.

In an alternative embodiment, when the cancer is chondrosarcoma, the further chemotherapeutic is Gemcitabine.

In a further embodiment, when the cancer is ovarian cancer, the further chemotherapeutic is selected from Doxorubicin, Gemcitabine, Geldanamycin, Roscovitine, Rapamycin and 5-FU or pro-drugs thereof.

In a yet further embodiment, when the cancer is prostate cancer, the further chemotherapeutic is selected from Doxorubicin, Geldanamycin and Roscovitine

In another embodiment, when the cancer is breast cancer, the further chemotherapeutic is 5-FU or pro-drugs thereof.

In an alternative embodiment, when the cancer is liver cancer, the further chemotherapeutic is selected from Gemcitabine, Geldanamycin, Roscovitine and Rapamycin.

In a thirteenth aspect of the invention there is provided a kit of parts comprising:

(i) a composition as defined in the first embodiment; and
(ii) a further chemotherapeutic agent.

The kit may optionally comprise:

(iii) means of administering (i) and (ii) to a patient, wherein the administration may be at the same time or in succession.

Preferably the further chemotherapeutic agent of the kit is selected from Doxorubicin, Gemcitabine, Roscovitine, Rapamycin, 5-FU, PARP inhibitors, kinase inhibitors including PIM kinase inhibitors and MAP kinase inhibitors and Hsp90 inhibitors (including Geldanamycin), proteasome inhibitors (including Bortezomib) and HDAC inhibitors (including SAHA); prodrugs thereof.

The kit may also comprise instructions for use.

Examples embodying certain aspects of the invention will now be described with reference to the following figures in which:

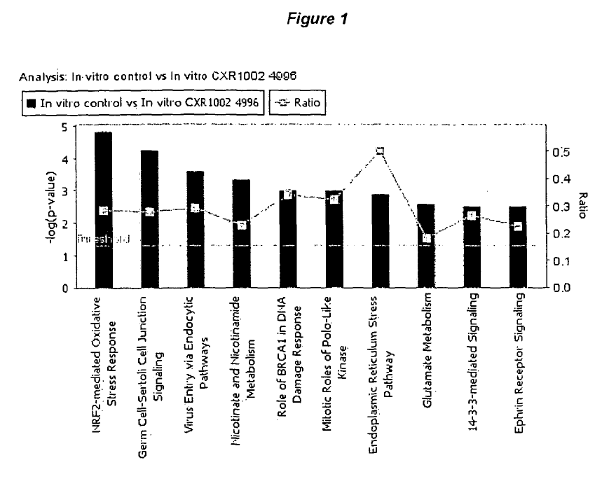

FIG. 1 shows the 10 canonical (classical) pathways that were most over-represented in the signature list of PANC-1 cells in vitro treated with CXR1002 for 24 hrs relative to representation of these genes in the Ingenuity Database. (Accessed using Ingenuity Pathway Analysis (IPA) software available from Ingenuity Systems, Inc. (Redwood City Calif., USA)). P− values represent the likelihood that the association between the canonical pathways and the genes in the signature lists is due to random chance. The P-value is calculated with a right-tailed Fisher's Exact Test. The ratio represents the number of genes in a canonical pathway that are found in the signature lists divided by the total number of genes in the pathway.

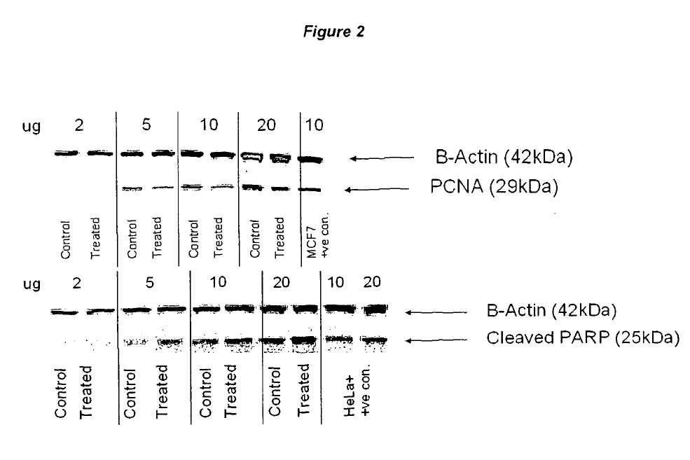

FIG. 2 shows the changes in protein levels for PCNA (top) and cleaved PARP (bottom) in CXR1002-treated PANC-1 cells. PCNA is a marker for cell proliferation and cleaved PARP is representative of caspase cleavage and apoptosis. PANC-1 cells were exposed to CXR1002 at 450 μM concentration (Treated) for 24 hrs or DMSO vehicle (Control). Western blot analysis was performed with increasing amounts of protein, ranging between 2 and 20 μg (lanes 1-8). Positive control protein was derived from MCF7 cells (PCNA blot, lane 9) or from HeLa cells treated with staurosporine for 3 hours (Cleaved PARP blot, lanes 9, 10). Levels of total β-Actin are shown as a control for protein loading. Treated cells show increased cleaved PARP and reduced PCNA levels, indicating increased apoptosis and reduced proliferation respectively.

FIG. 3 shows the effects of CXR1002 on HT29 xenografts. Filled diamonds represent mean tumour volumes for animals treated with 25 mg/kg CXR1002 over time compared to those for saline treated control animals (empty squares). Tumour volumes were plotted using Graph Pad Prism software.

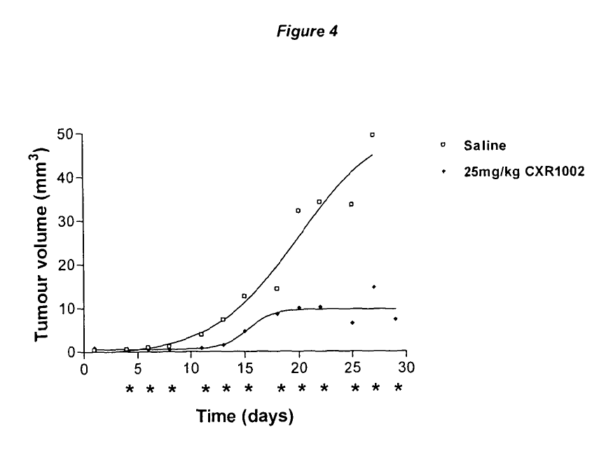

FIG. 4 shows the effects of CXR1002 on PC-3 xenografts. Filled diamonds represent mean tumour volumes for animals treated with 25 mg/kg CXR1002 over time compared to those for saline treated control animals (empty squares). Tumour volumes were plotted using Graph Pad Prism software.

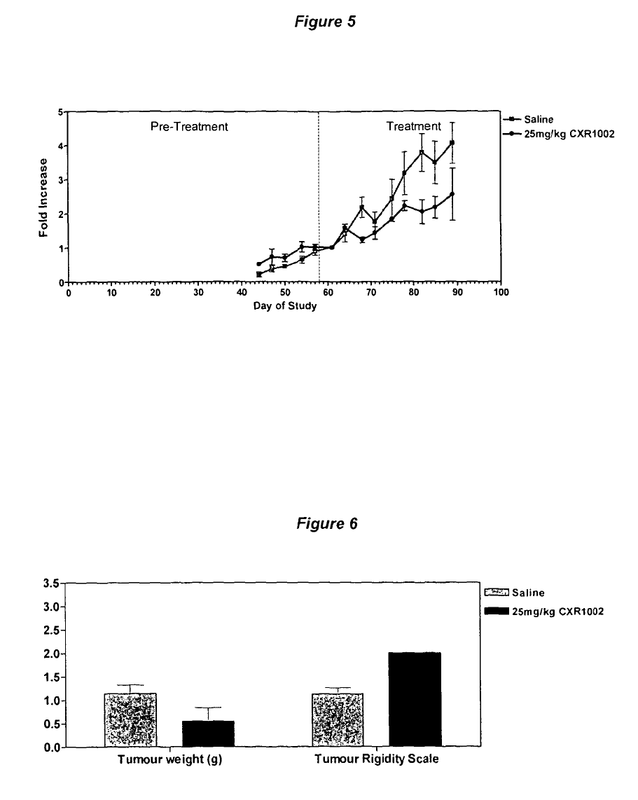

FIG. 5 shows the effects of CXR1002 on PANC-1 tumours relative to the first day of treatment. Black line indicates the fold increase in tumour size for animals treated with 25 mg/kg CXR1002 over time compared to those for saline treated control animals (grey line).

FIG. 6 shows the effects of CXR1002 on PANC-1 tumour weights and tumour rigidity.

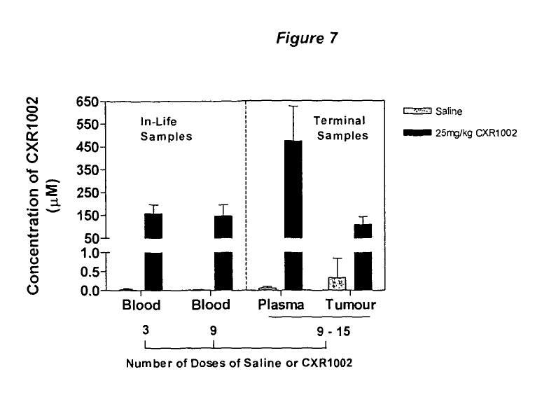

FIG. 7 shows the concentrations of CXR1002 in blood during the in-life stage of treatment, and in plasma and tumour tissue in terminal samples in treated (dark grey) versus control (light grey) animals.

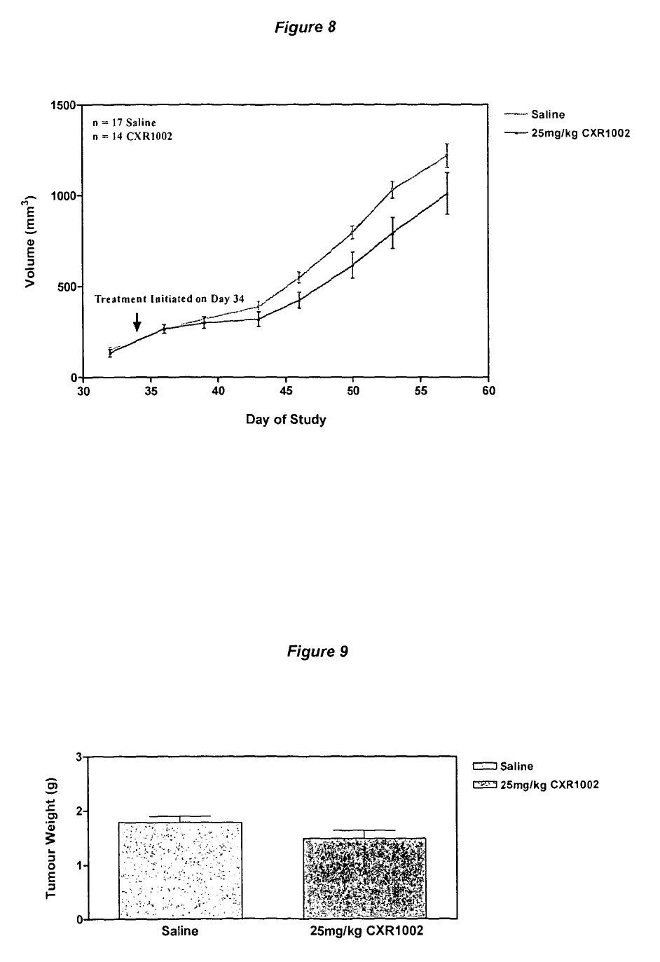

FIG. 8 shows the effects of CXR1002 on HepG2 xenografts. Dark grey represents mean tumour volumes for animals treated with 25 mg/kg CXR1002 over time compared to those for saline treated control animals (light grey).

FIG. 9 shows the effects on tumour weight of HepG2 xenografts. Dark grey represents combined tumour weights for animals treated with 25 mg/kg CXR1002 over time compared to those for saline treated control animals (light grey).

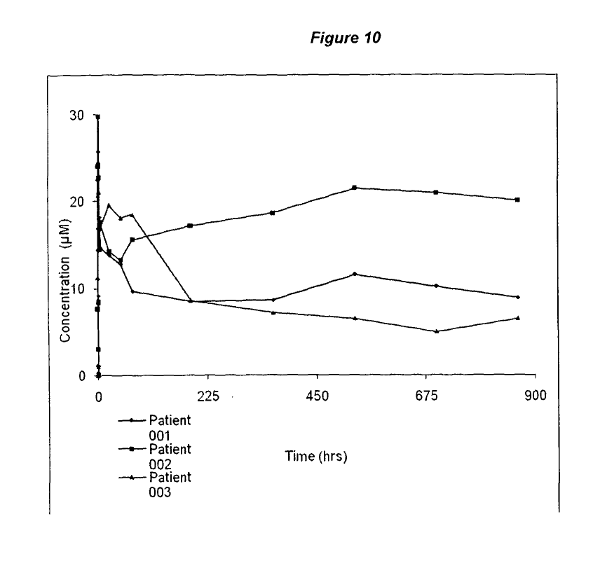

FIG. 10 shows the plasma levels of CXR1002 over 6 weeks in a cohort of 3 patients after a single 50 mg dose.

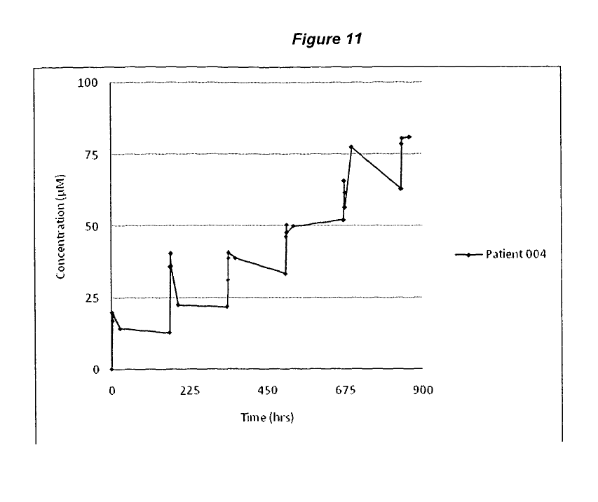

FIG. 11 shows accumulating levels of CXR1002 following a repeat weekly 50 mg dose to for 6 weeks in a single patient.

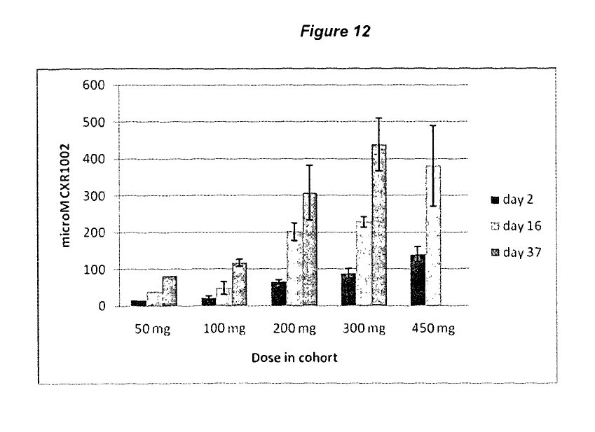

FIG. 12 shows the increase in exposure with increasing dose level (50-450 mg) and duration (2-37 days) of a repeat weekly dose of CXR1002.

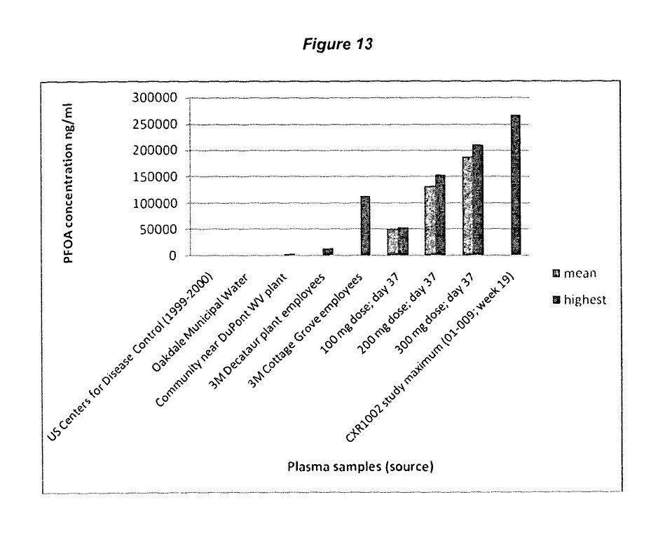

FIG. 13 shows a comparison of the exposure levels of PFOA in occupationally exposed workers compared to the exposure levels of CXR1002 in patients participating in the clinical trial.

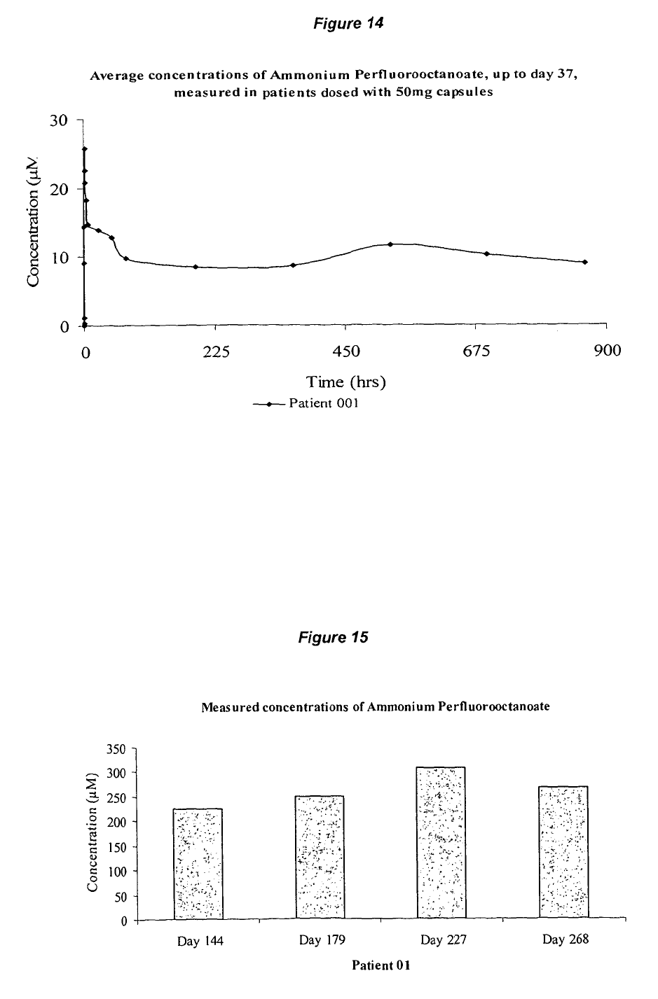

FIG. 14 shows the average concentrations of APFO measured over 37 days for 3 patients dosed with a single dose of 50 mg of CXR1002.

FIG. 15 shows measured concentrations of APFO in patient 1 at 4 time points (days 144, 179, 227, 268) following a single dose of 50 mg of CXR1002.

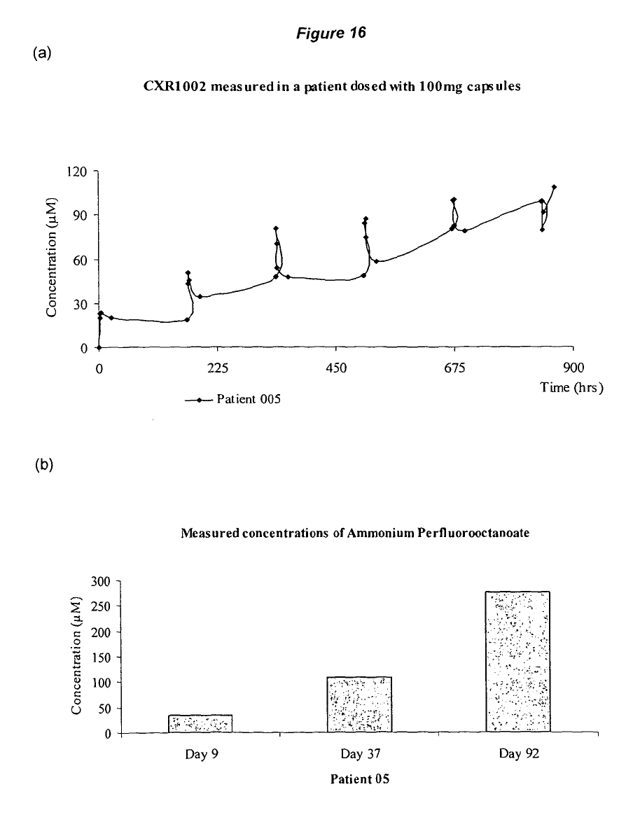

FIG. 16 shows (a) accumulating levels of CXR1002 following a repeat weekly 100 mg dose for 6 weeks in patient 005 and (b) measured concentrations of APFO at 3 specific time points.

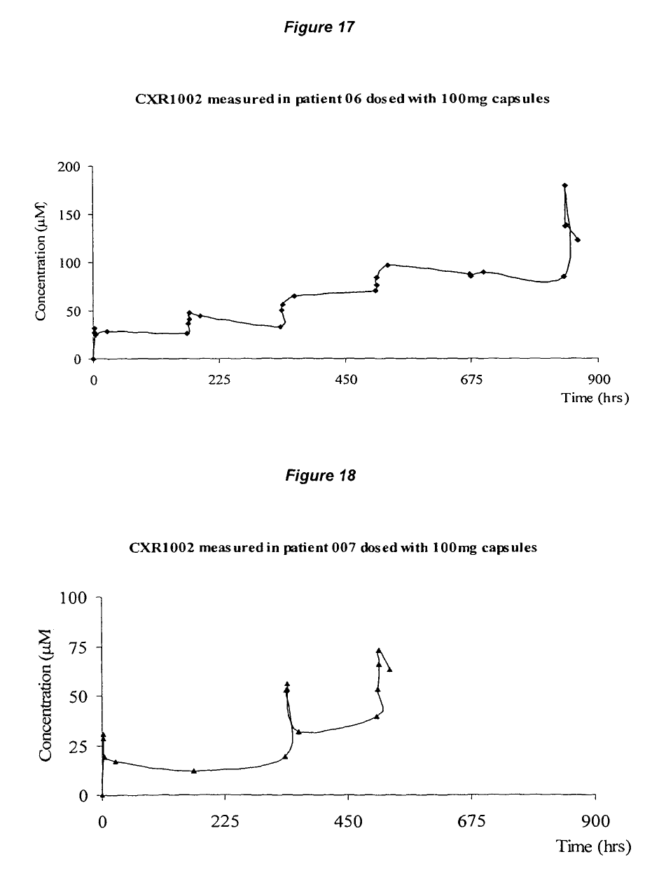

FIG. 17 shows accumulating levels of CXR1002 following a repeat weekly 100 mg dose for 6 weeks in patient 006.

FIG. 18 shows accumulating levels of CXR1002 following a repeat weekly 100 mg dose for 6 weeks in patient 007.

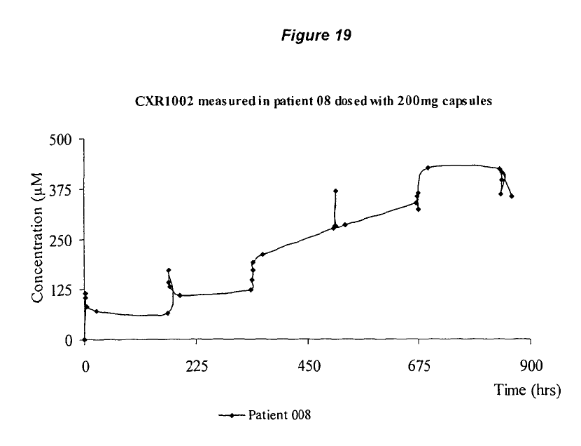

FIG. 19 shows accumulating levels of CXR1002 following a repeat weekly 200 mg dose for 6 weeks in patient 008.

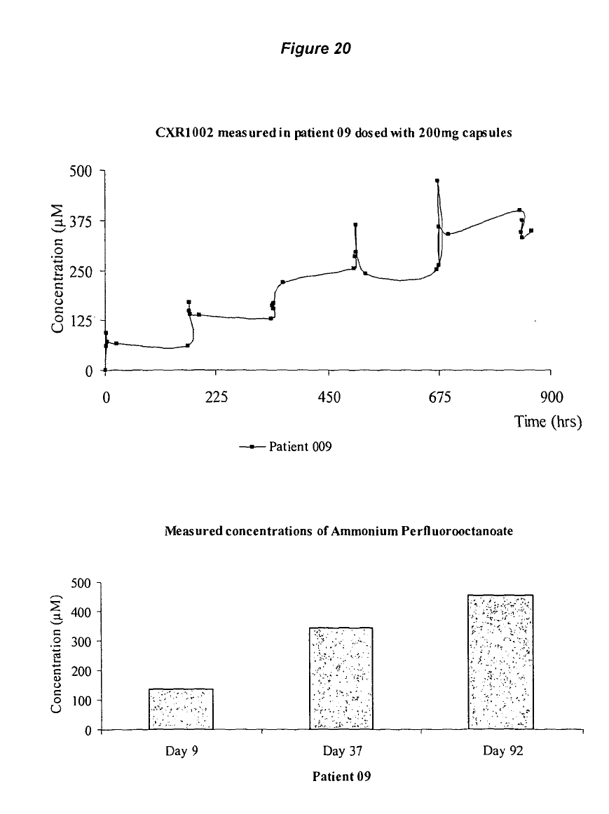

FIG. 20 shows (a) accumulating levels of CXR1002 following a repeat weekly 200 mg dose for 6 weeks in patient 009 and (b) measured concentrations of CXR1002 at 3 specific time points.

FIG. 21 shows accumulating levels of CXR1002 following a repeat weekly 200 mg dose for 6 weeks in patient 010.

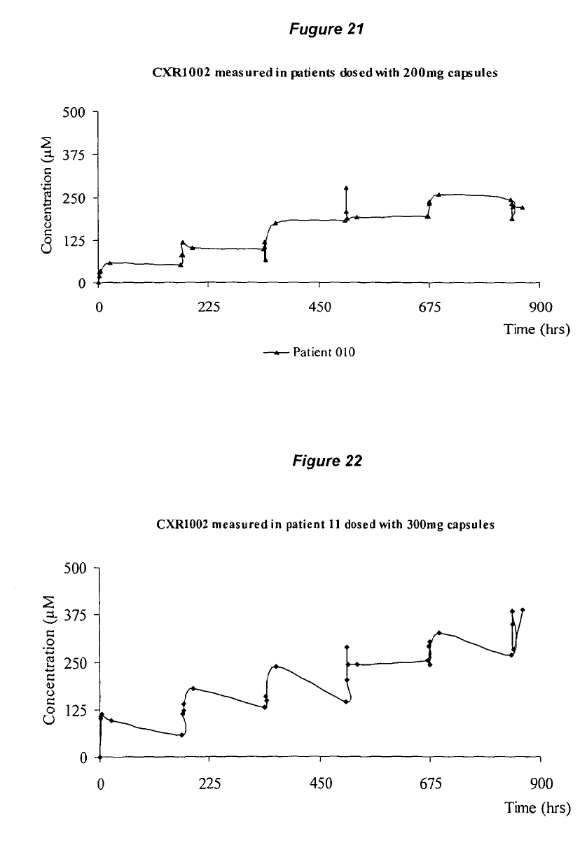

FIG. 22 shows accumulating levels of CXR1002 following a repeat weekly 300 mg dose for 6 weeks in patient 011.

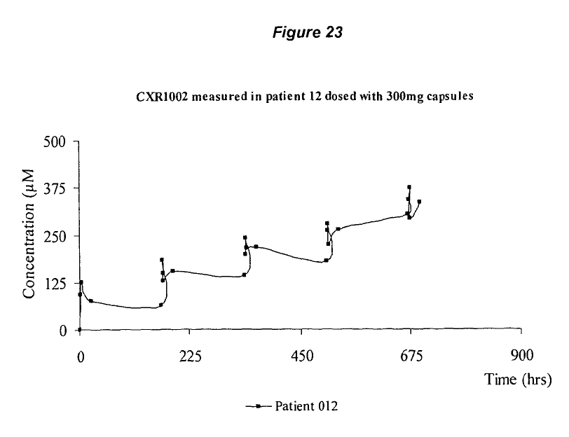

FIG. 23 shows accumulating levels of CXR1002 following a repeat weekly 300 mg dose for 6 weeks in patient 012.

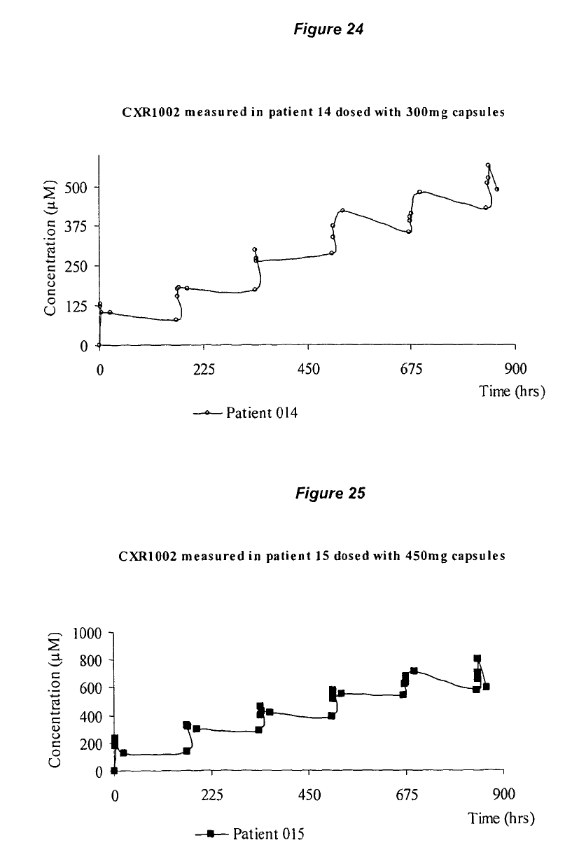

FIG. 24 shows accumulating levels of CXR1002 following a repeat weekly 450 mg dose for 6 weeks in patient 014.

FIG. 25 shows accumulating levels of CXR1002 following a repeat weekly 450 mg dose for 6 weeks in patient 015.

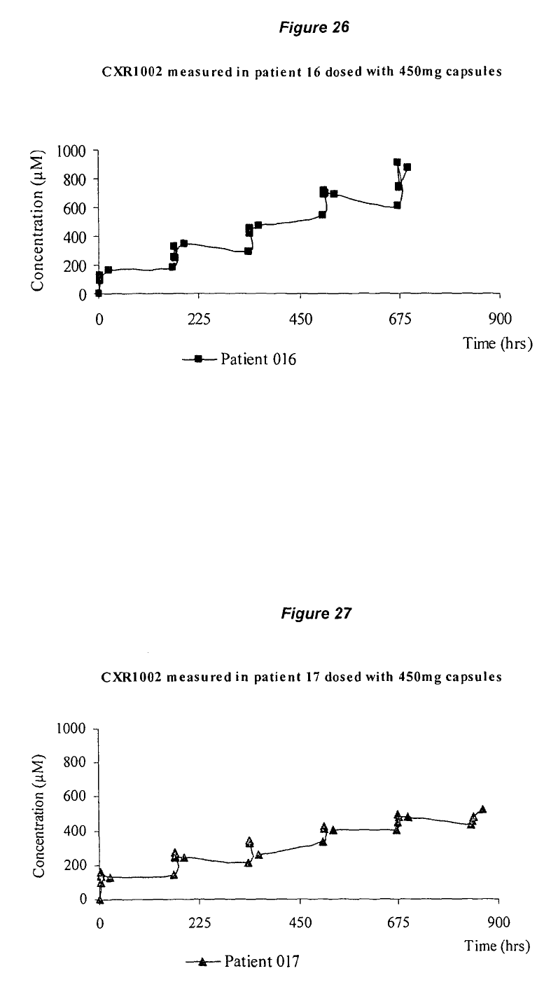

FIG. 26 shows accumulating levels of CXR1002 following a repeat weekly 450 mg dose for 6 weeks in patient 016.

FIG. 27 shows accumulating levels of CXR1002 following a repeat weekly 450 mg dose for 6 weeks in patient 017.

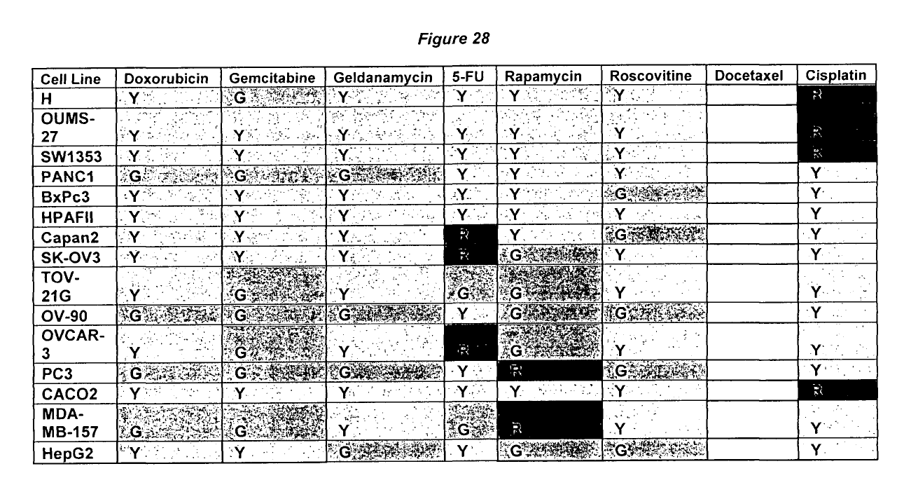

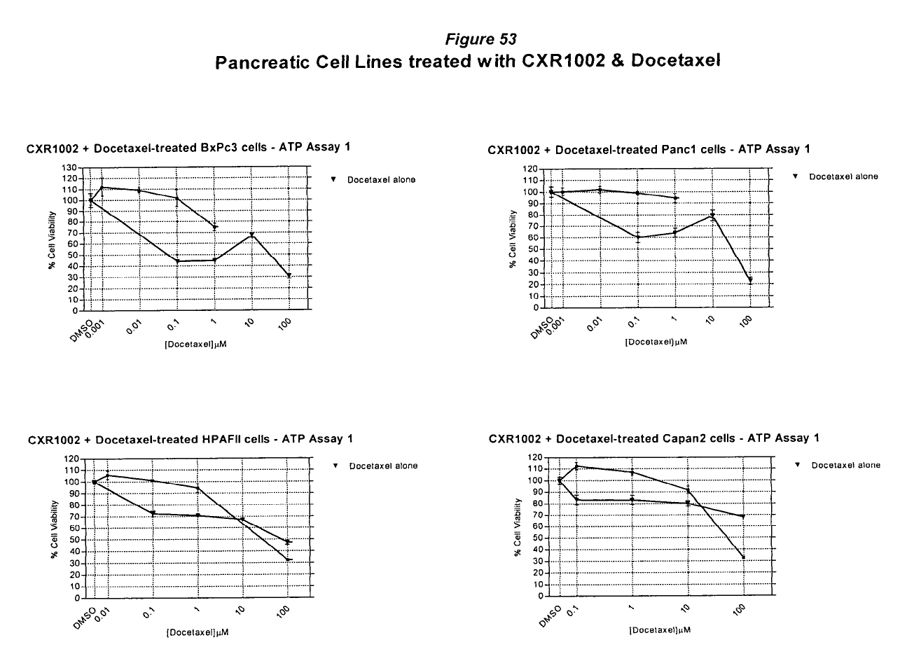

FIG. 28 shows a summary of the cytotoxicity assay results for test items combined with CXR1002 compared to treatment with test items alone. Medium grey (G)—more sensitive; light grey (y)—no change: dark grey (R)—possible decrease in sensitivity. Docetaxel when used alone in cytotoxicity assays gave unexpected results with most of the cell lines, as shown in the FIGS. 53-56. The same results were obtained when the assays were repeated (data not shown). When used in combination with CXR1002, curves more usually associated with cytotoxicity assays were obtained (plotted as squares in graphs in FIGS. 53-56).

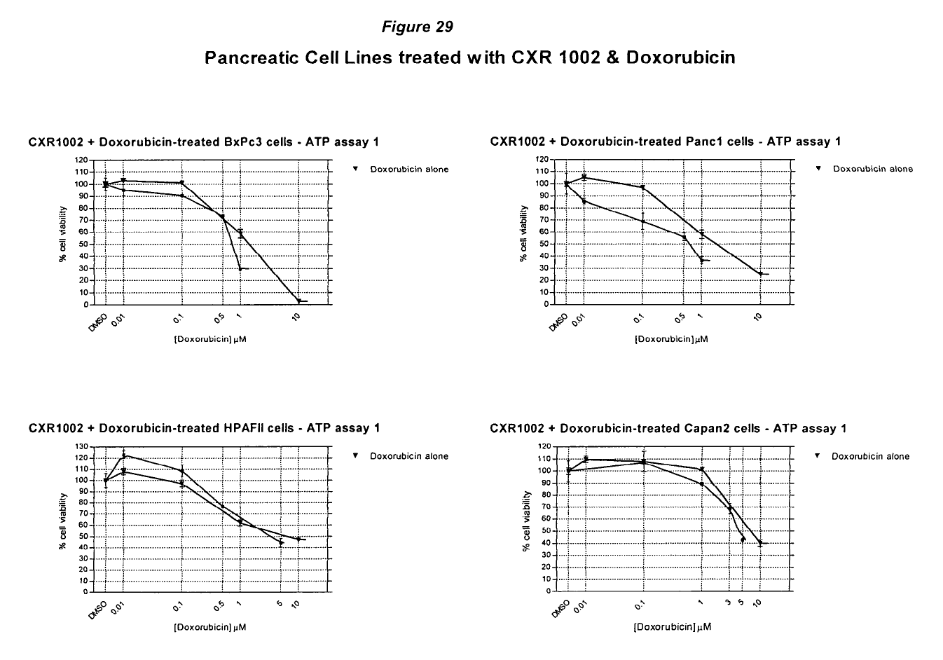

FIG. 29 shows cytotoxicity plots for pancreatic cell lines treated with CXR1002 and Doxorubicin. Plots show percentage cell viability of cells treated in combination (squares) compared to Doxorubicin alone (triangles).

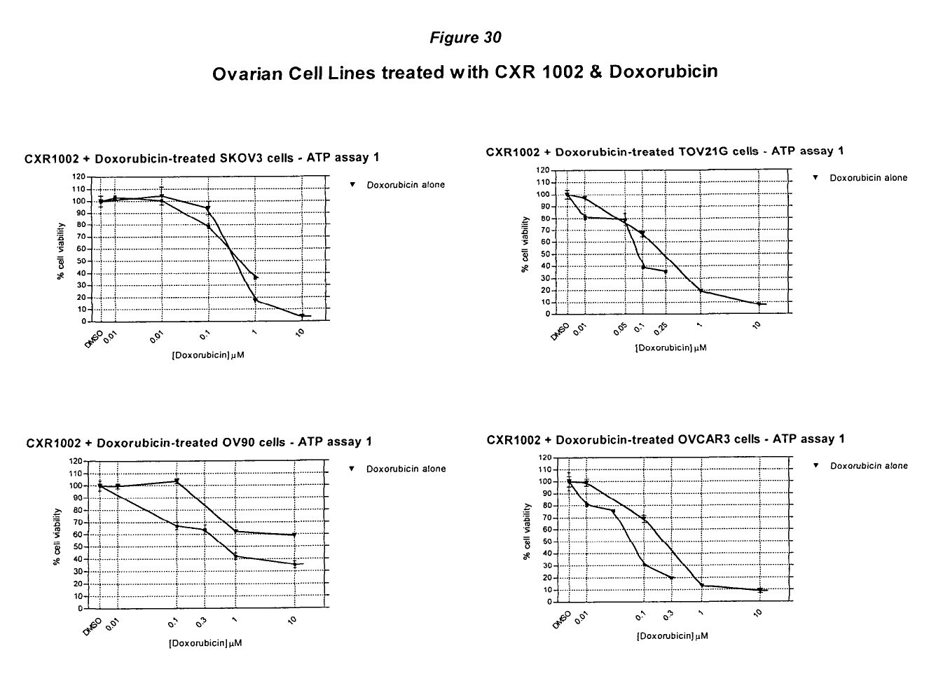

FIG. 30 shows cytotoxicity plots for ovarian cell lines treated with CXR1002 and Doxorubicin. Plots show percentage cell viability of cells treated in combination (squares) compared to Doxorubicin alone (triangles).

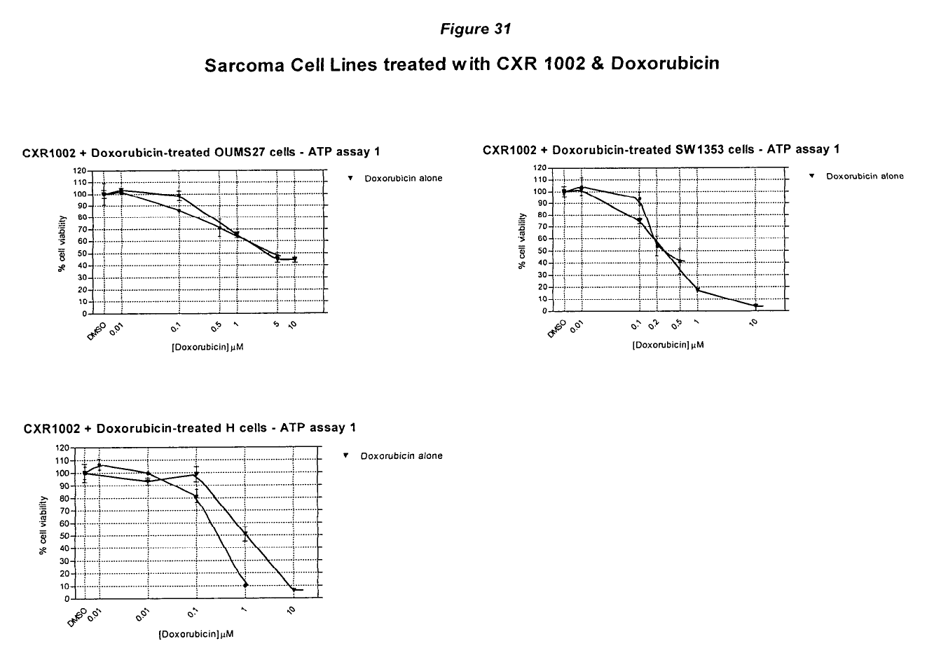

FIG. 31 shows cytotoxicity plots for sarcoma cell lines treated with CXR1002 and Doxorubicin. Plots show percentage cell viability of cells treated in combination (squares) compared to Doxorubicin alone (triangles).

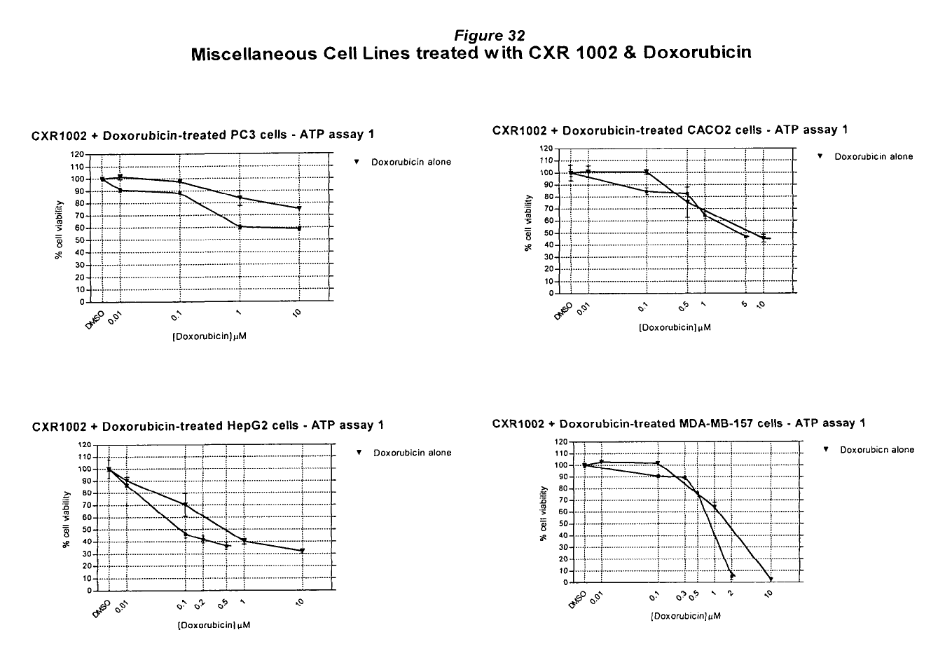

FIG. 32 shows cytotoxicity plots for further cell lines treated with CXR1002 and Doxorubicin. Plots show percentage cell viability of cells treated in combination (squares) compared to Doxorubicin alone (triangles).

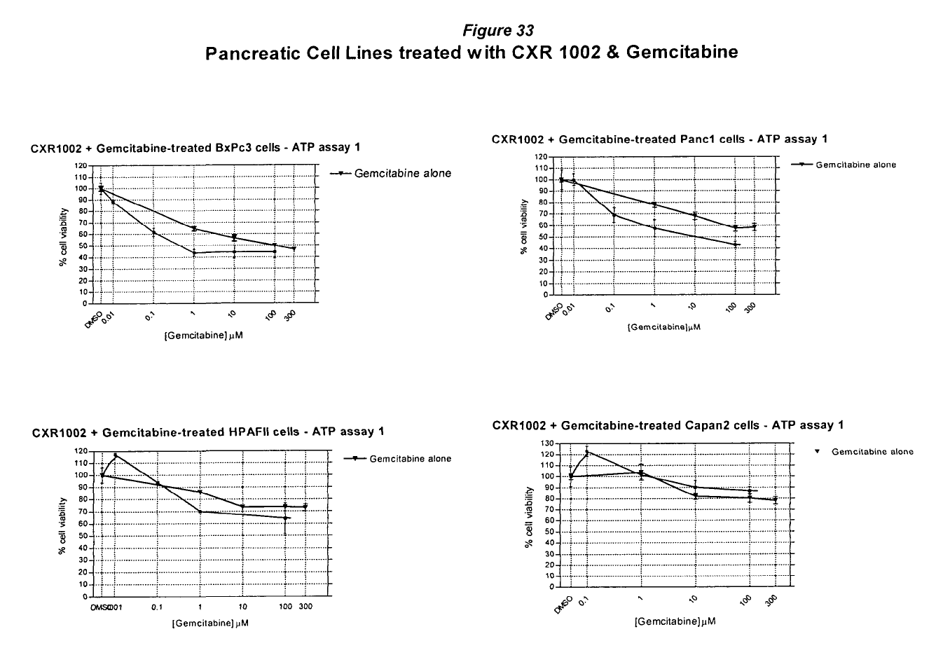

FIG. 33 shows cytotoxicity plots for pancreatic cell lines treated with CXR1002 and Gemcitabine. Plots show percentage cell viability of cells treated in combination (squares) compared to Gemcitabine alone (triangles).

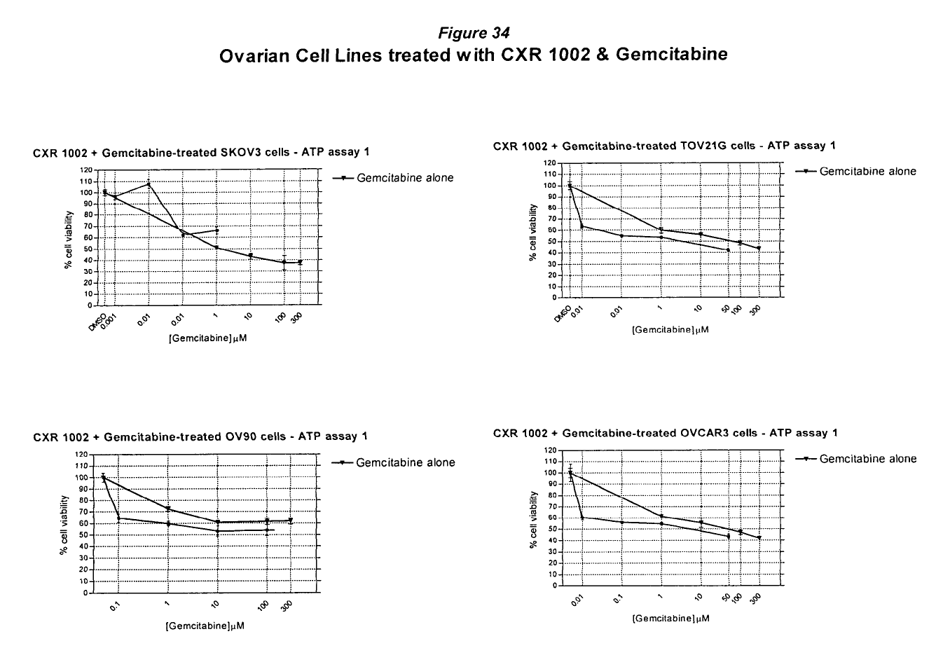

FIG. 34 shows cytotoxicity plots for ovarian cell lines treated with CXR1002 and Gemcitabine. Plots show percentage cell viability of cells treated in combination (squares) compared to Gemcitabine alone (triangles).

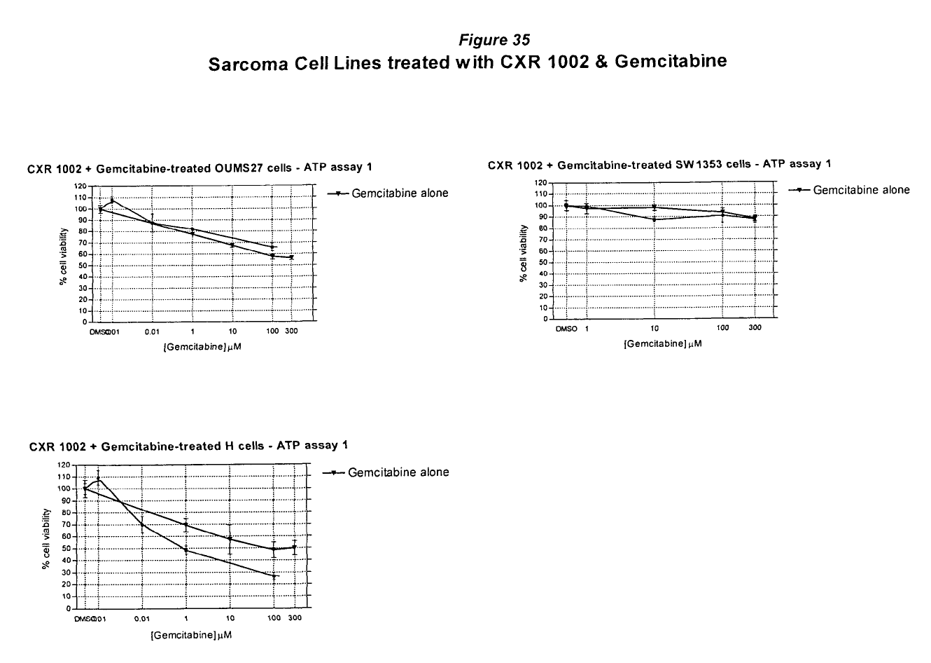

FIG. 35 shows cytotoxicity plots for sarcoma cell lines treated with CXR1002 and Gemcitabine. Plots show percentage cell viability of cells treated in combination (squares) compared to Gemcitabine alone (triangles).

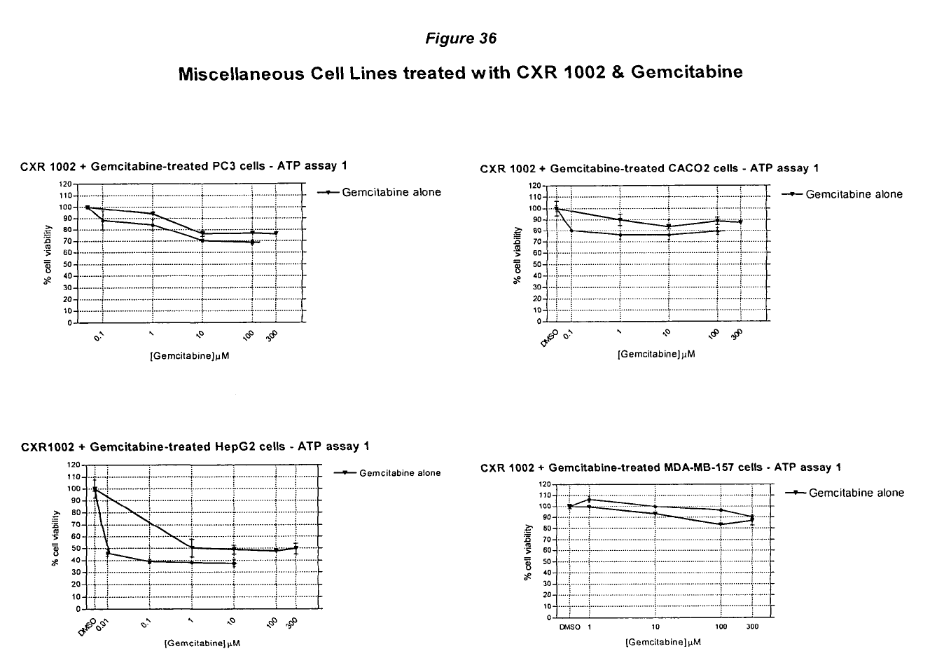

FIG. 36 shows cytotoxicity plots for further cell lines treated with CXR1002 and Gemcitabine, Plots show percentage cell viability of cells treated in combination (squares) compared to Gemcitabine alone (triangles).

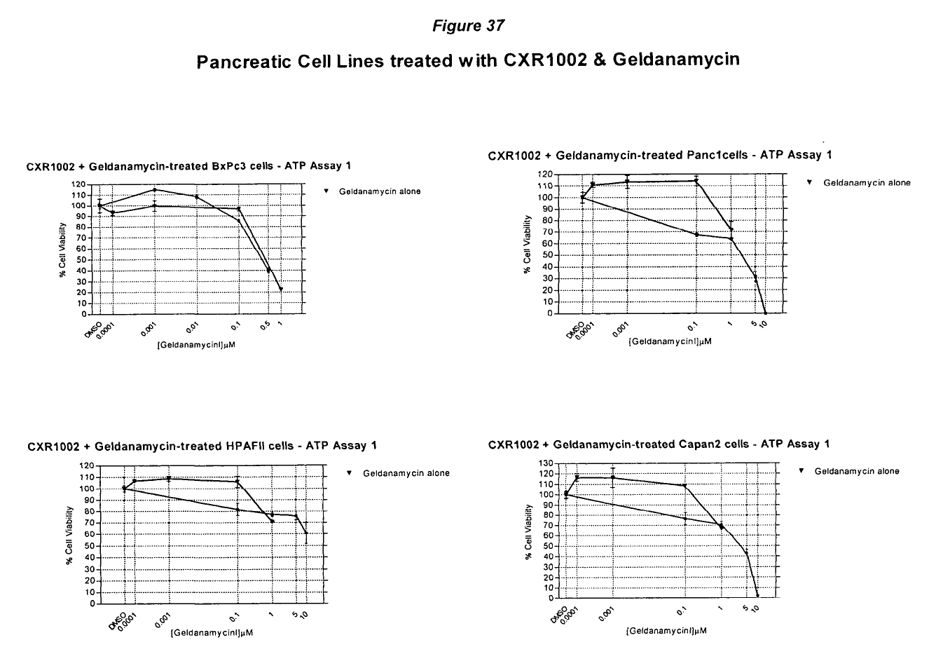

FIG. 37 shows cytotoxicity plots for pancreatic cell lines treated with CXR1002 and Geldanamycin. Plots show percentage cell viability of cells treated in combination (squares) compared to Geldanamycin alone (triangles).

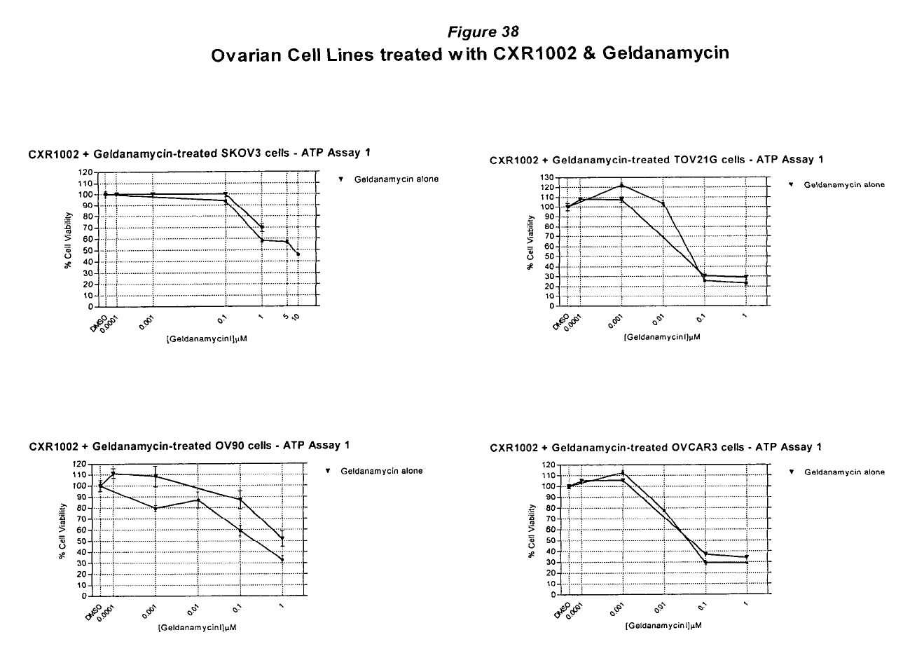

FIG. 38 shows cytotoxicity plots for ovarian cell lines treated with CXR1002 and Geldanamycin. Plots show percentage cell viability of cells treated in combination (squares) compared to Geldanamycin alone (triangles).

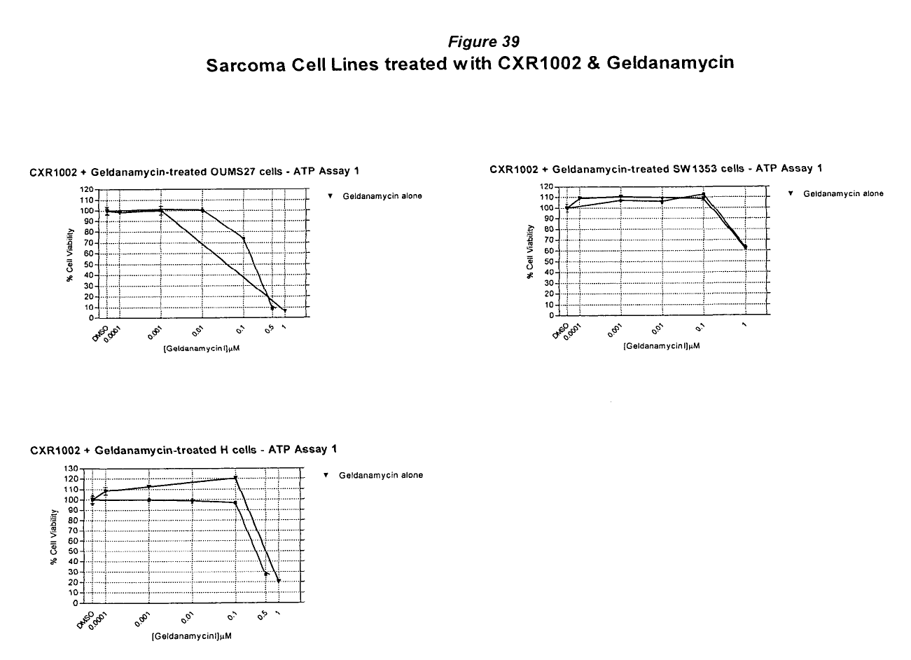

FIG. 39 shows cytotoxicity plots for sarcoma cell lines treated with CXR1002 and Geldanamycin. Plots show percentage cell viability of cells treated in combination (squares) compared to Geldanamycin alone (triangles).

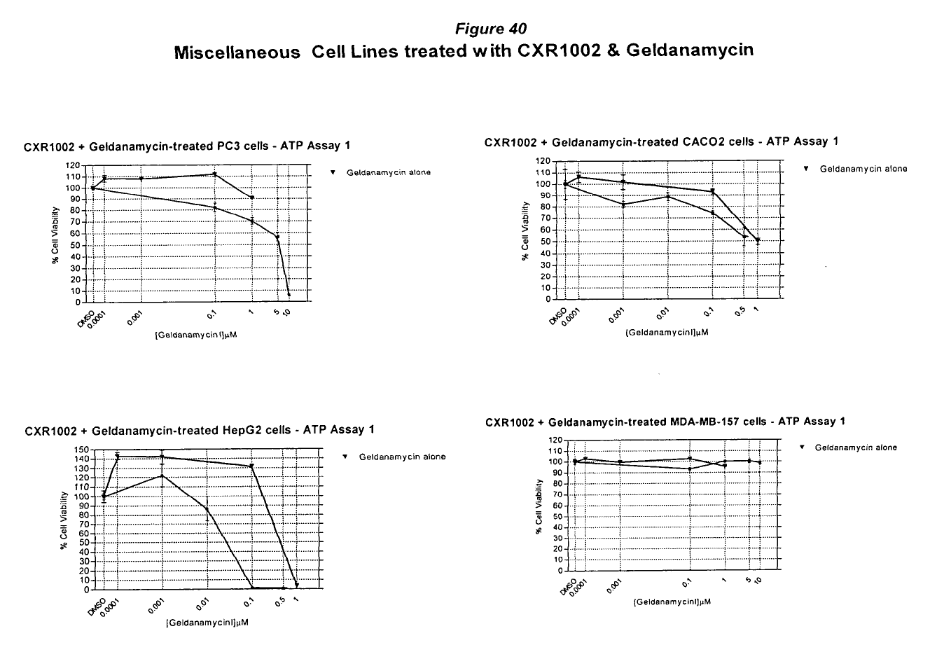

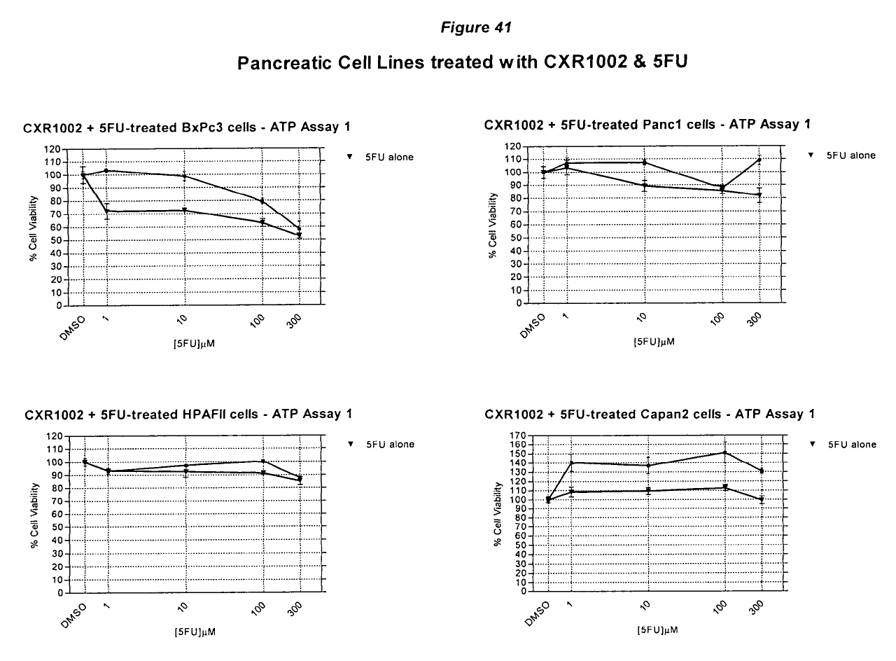

FIG. 40 shows cytotoxicity plots for further cell lines treated with CXR1002 and Geldanamycin. Plots show percentage cell viability of cells treated in combination (squares) compared to Geldanamycin alone (triangles).

FIG. 41 shows cytotoxicity plots for pancreatic cell lines treated with CXR1002 and 5FU. Plots show percentage cell viability of cells treated in combination (squares) compared to 5FU alone (triangles).

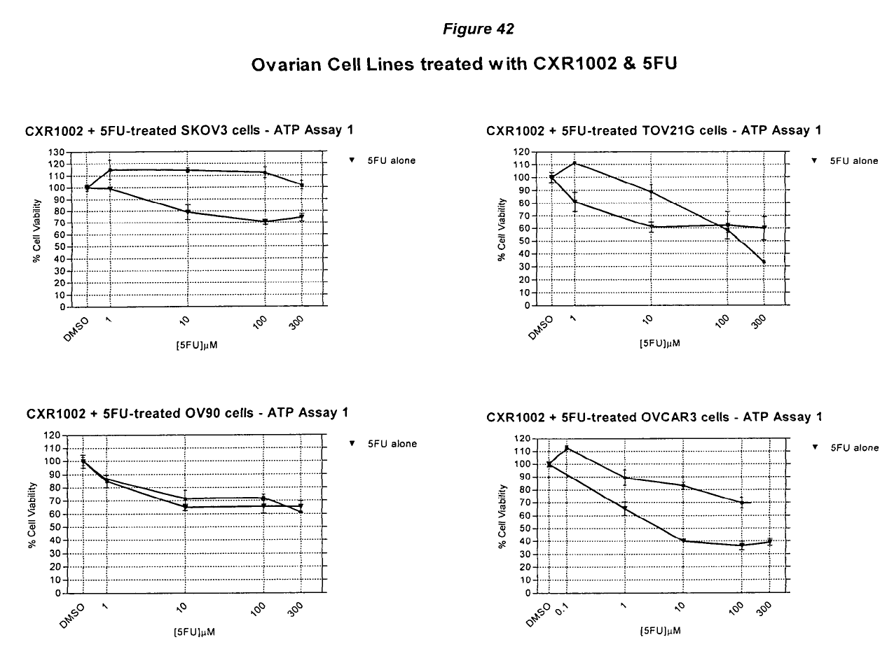

FIG. 42 shows cytotoxicity plots for ovarian cell lines treated with CXR1002 and 5FU. Plots show percentage cell viability of cells treated in combination (squares) compared to 5FU alone (triangles).

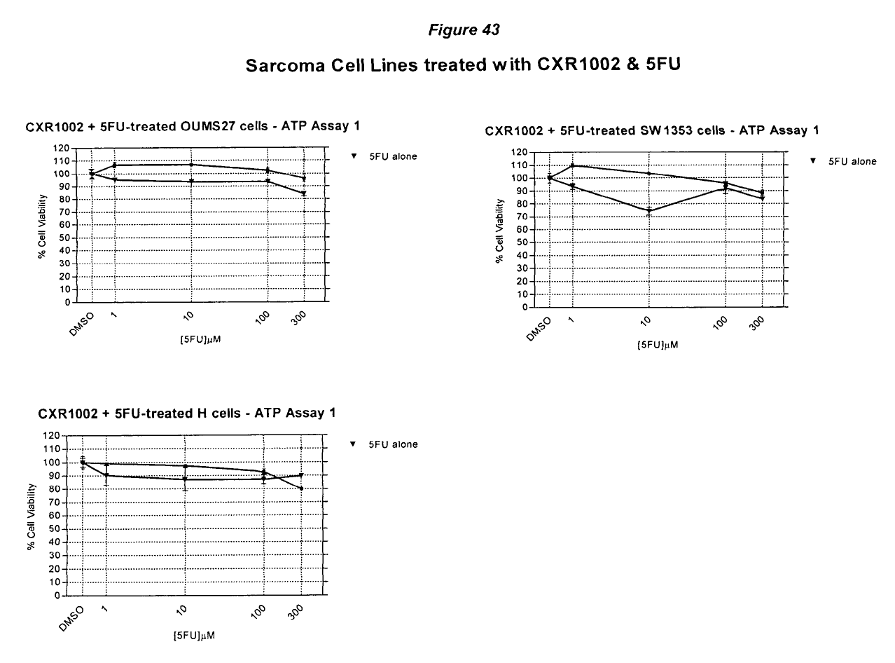

FIG. 43 shows cytotoxicity plots for sarcoma cell lines treated with CXR1002 and 5FU. Plots show percentage cell viability of cells treated in combination (squares) compared to 5FU alone (triangles).

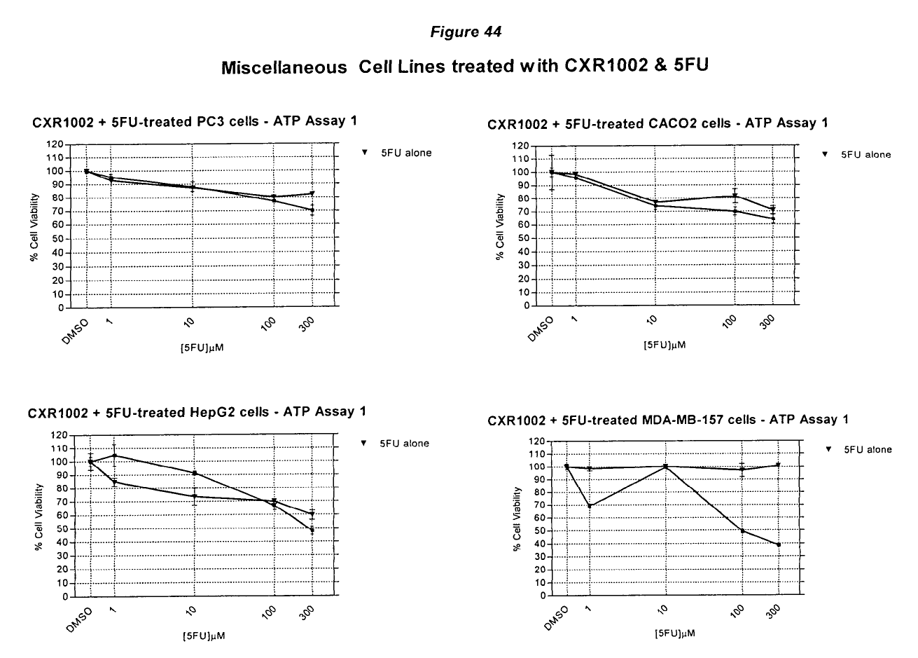

FIG. 44 shows cytotoxicity plots for further cell lines treated with CXR1002 and 5FU. Plots show percentage cell viability of cells treated in combination (squares) compared to 5FU alone (triangles).

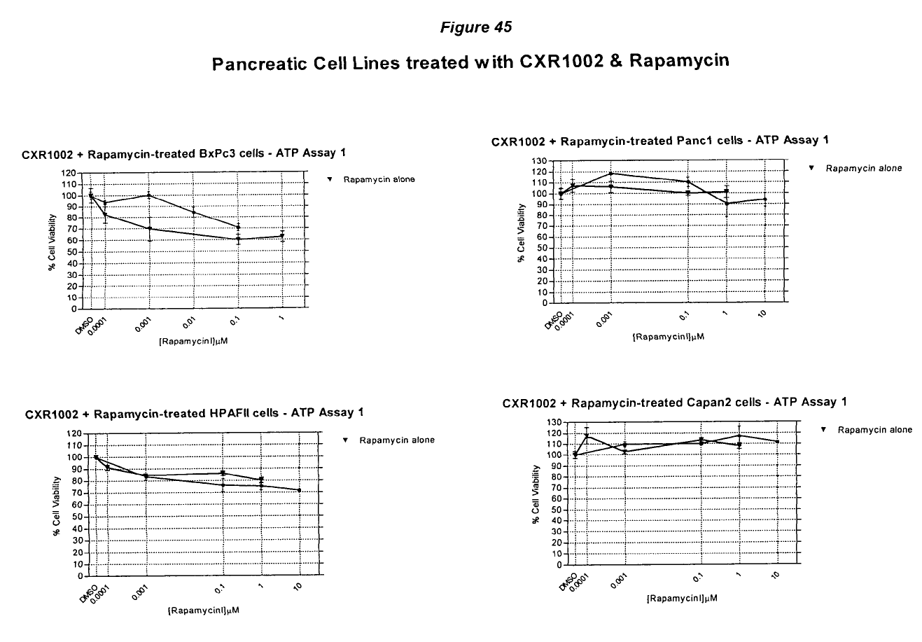

FIG. 45 shows cytotoxicity plots for pancreatic cell lines treated with CXR1002 and Rapamycin. Plots show percentage cell viability of cells treated in combination (squares) compared to Rapamycin alone (triangles).

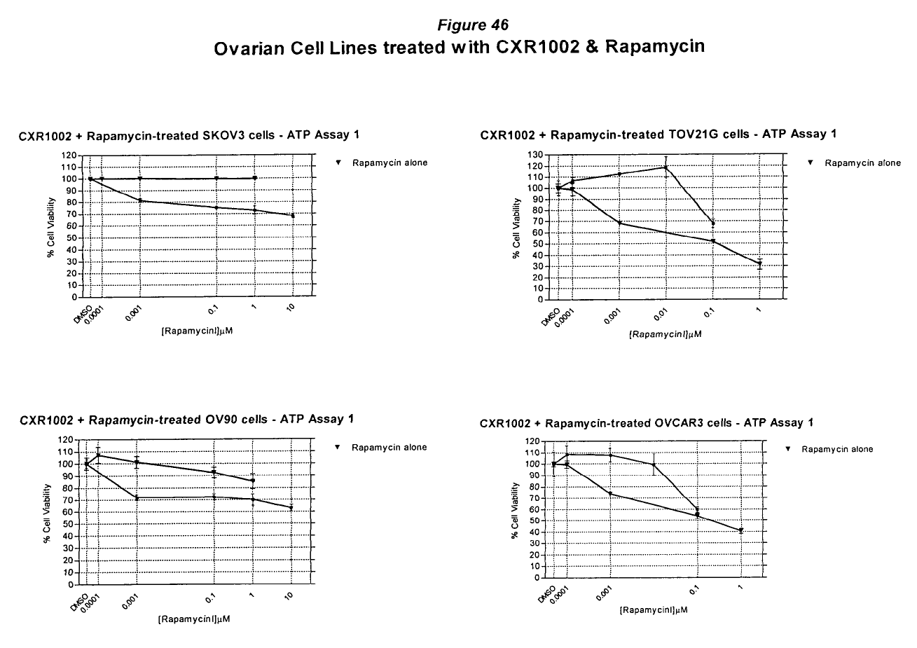

FIG. 46 shows cytotoxicity plots for ovarian cell lines treated with CXR1002 and Rapamycin. Plots show percentage cell viability of cells treated in combination (squares) compared to Rapamycin alone (triangles).

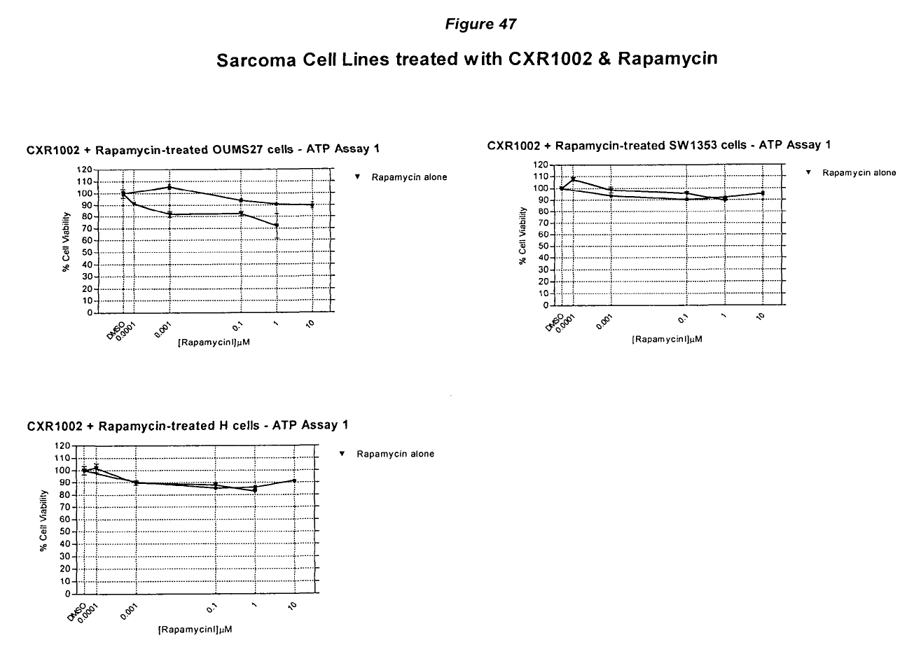

FIG. 47 shows cytotoxicity plots for sarcoma cell lines treated with CXR1002 and Rapamycin. Plots show percentage cell viability of cells treated in combination (squares) compared to Rapamycin alone (triangles).

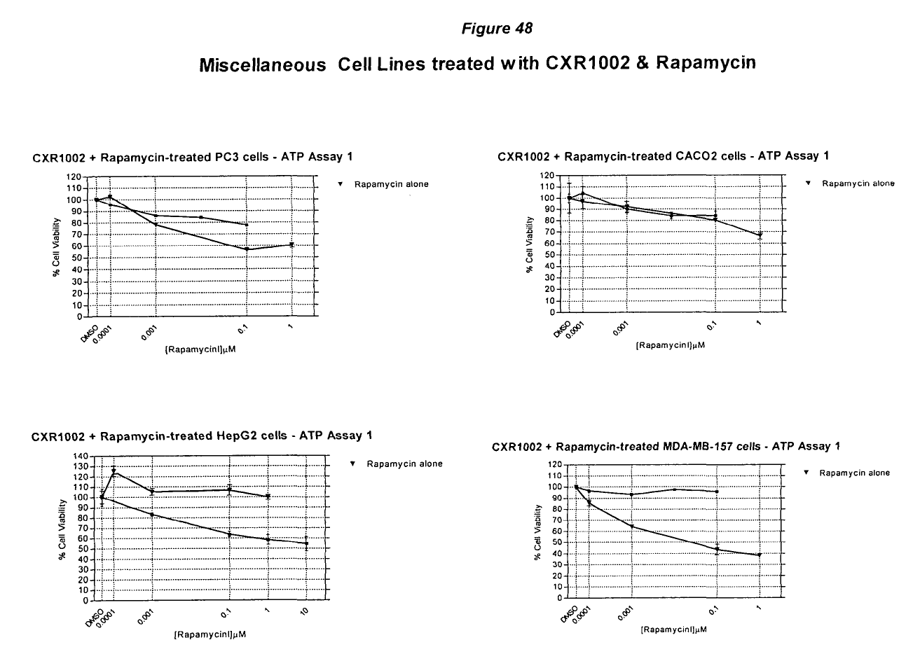

FIG. 48 shows cytotoxicity plots for further cell lines treated with CXR1002 and Rapamycin. Plots show percentage cell viability of cells treated in combination (squares) compared to Rapamycin alone (triangles).

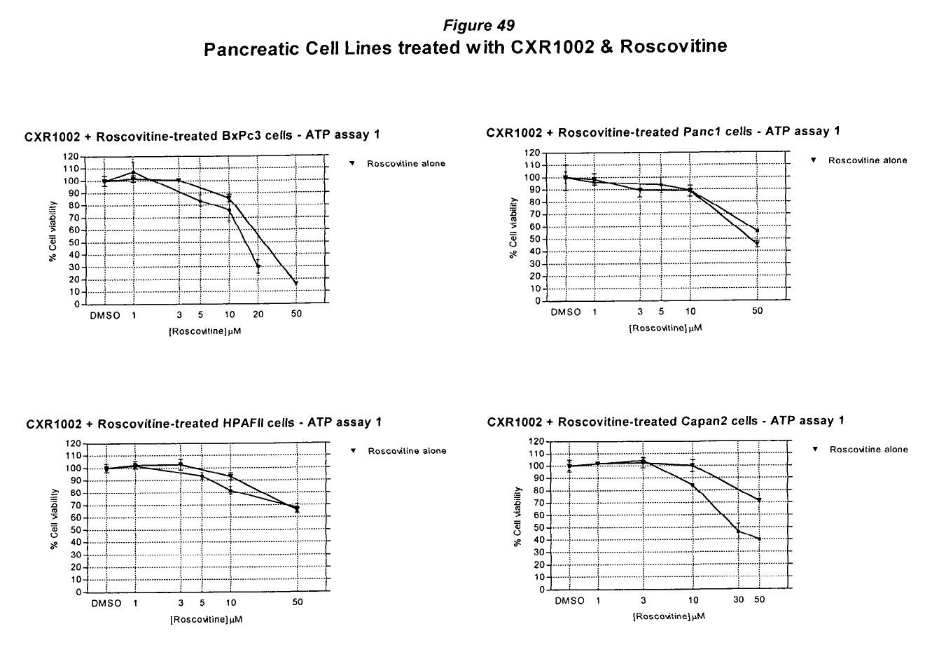

FIG. 49 shows cytotoxicity plots for pancreatic cell lines treated with CXR1002 and Roscovitine. Plots show percentage cell viability of cells treated in combination (squares) compared to Roscovitine alone (triangles).

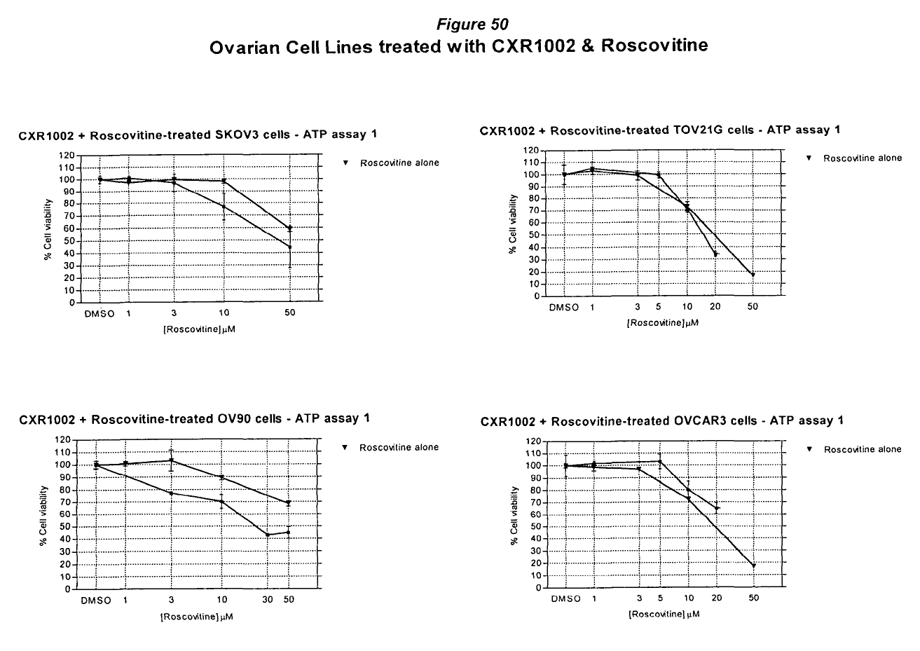

FIG. 50 shows cytotoxicity plots for ovarian cell lines treated with CXR1002 and Roscovitine. Plots show percentage cell viability of cells treated in combination (squares) compared to Roscovitine alone (triangles).

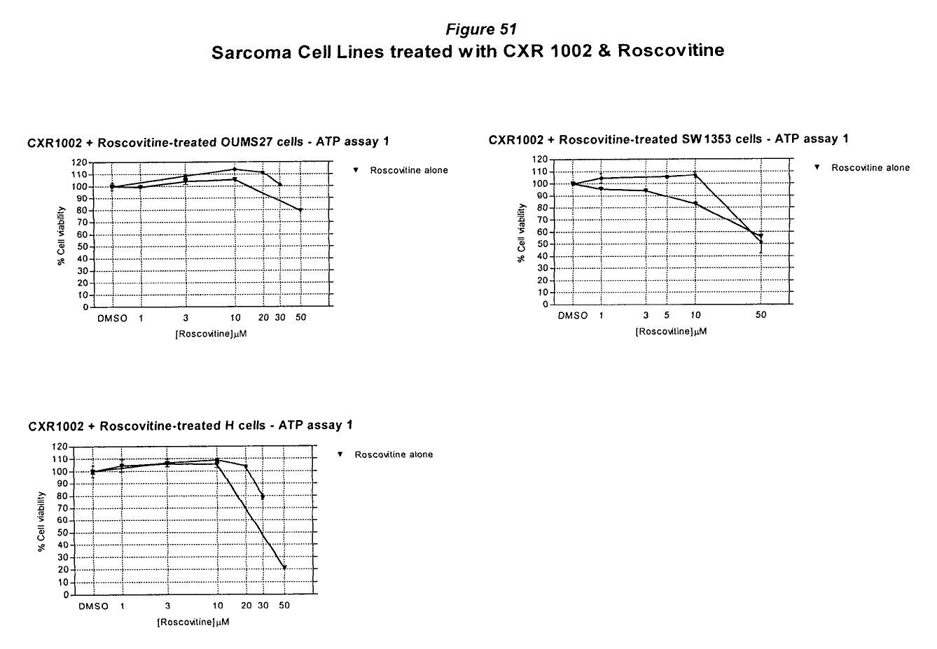

FIG. 51 shows cytotoxicity plots for sarcoma cell lines treated with CXR1002 and Roscovitine. Plots show percentage cell viability of cells treated in combination (squares) compared to Roscovitine alone (triangles).

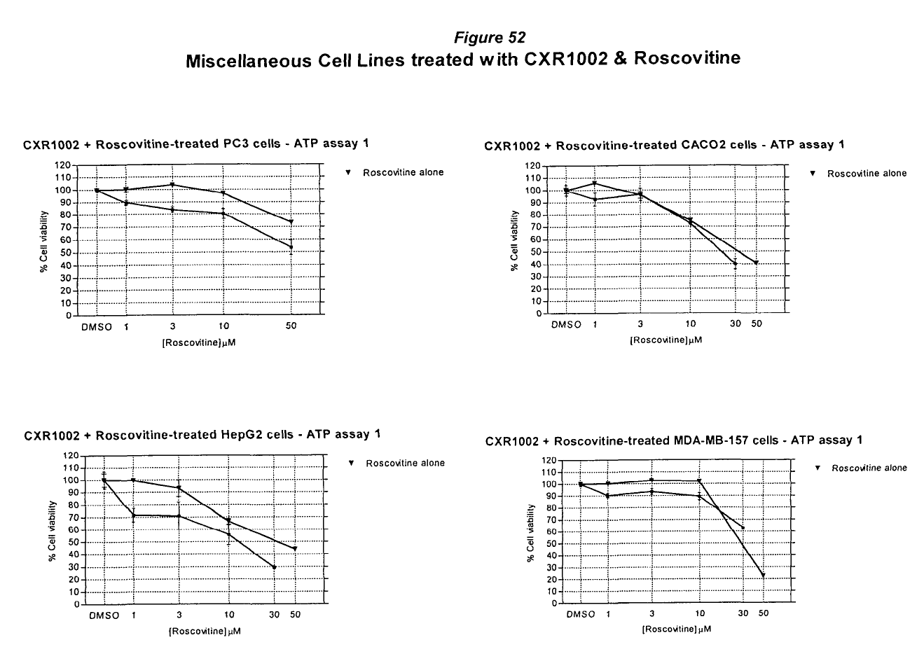

FIG. 52 shows cytotoxicity plots for further cell lines treated with CXR1002 and Roscovitine. Plots show percentage cell viability of cells treated in combination (squares) compared to Roscovitine alone (triangles).

FIG. 53 shows cytotoxicity plots for pancreatic cell lines treated with CXR1002 and Docetaxel. Plots show percentage cell viability of cells treated in combination (squares) compared to Docetaxel alone (triangles).

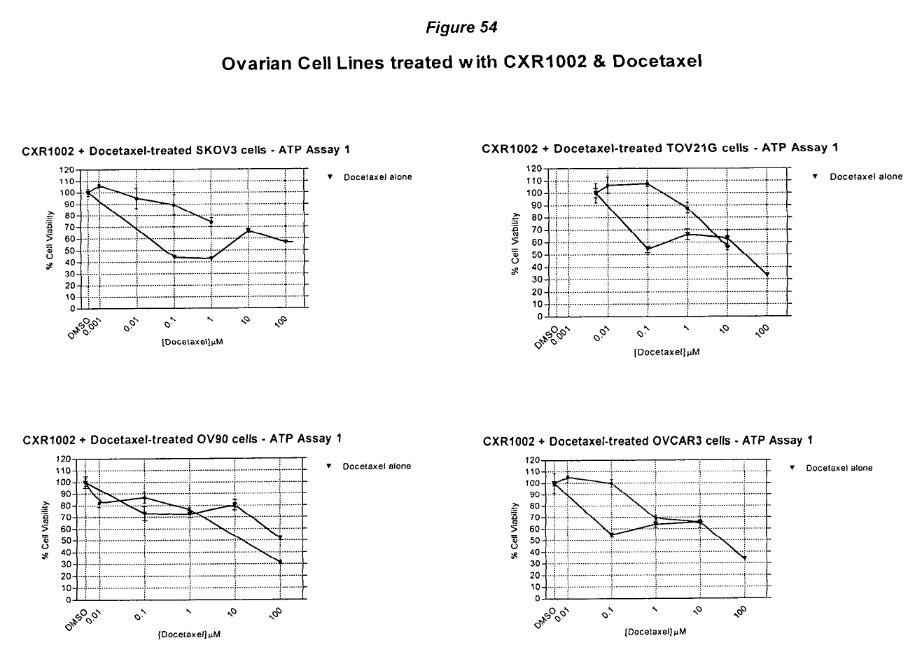

FIG. 54 shows cytotoxicity plots for ovarian cell lines treated with CXR1002 and Docetaxel. Plots show percentage cell viability of cells treated in combination (squares) compared to Docetaxel alone (triangles).

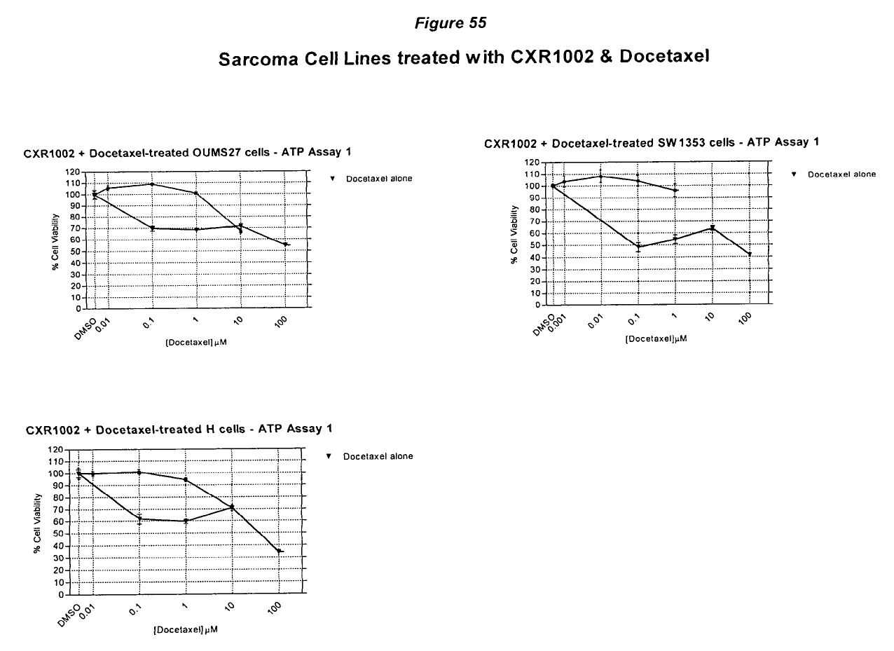

FIG. 55 shows cytotoxicity plots for sarcoma cell lines treated with CXR1002 and Docetaxel. Plots show percentage cell viability of cells treated in combination (squares) compared to Docetaxel alone (triangles).

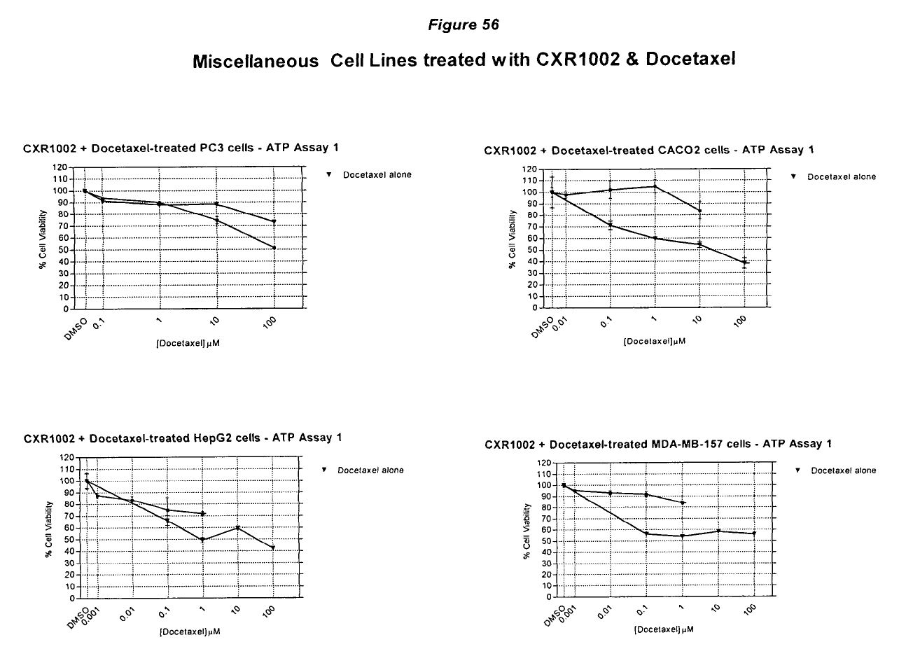

FIG. 56 shows cytotoxicity plots for further cell lines treated with CXR1002 and Docetaxel. Plots show percentage cell viability of cells treated in combination (squares) compared to Docetaxel alone (triangles).

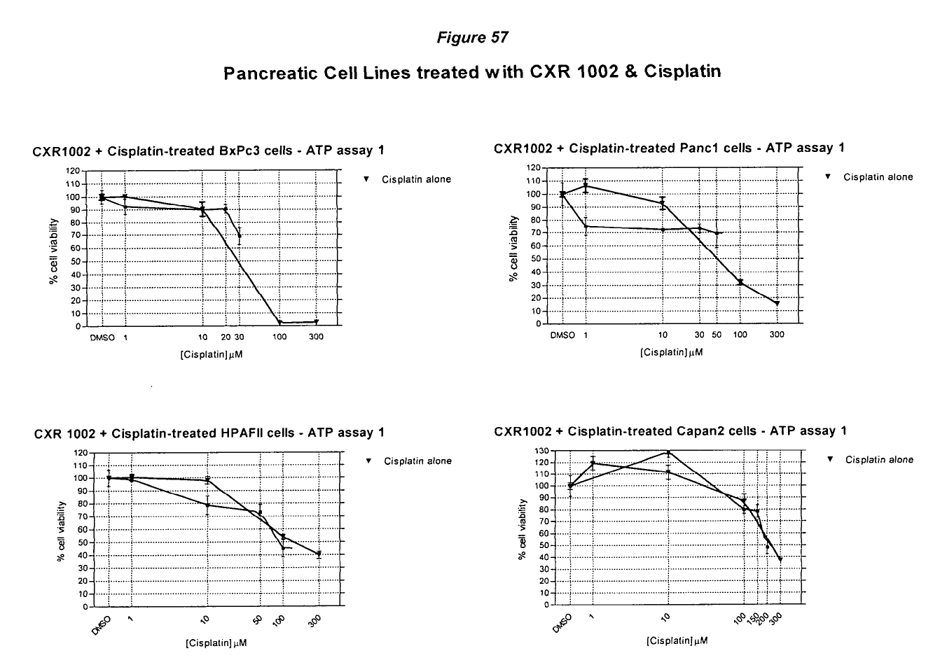

FIG. 57 shows cytotoxicity plots for pancreatic cell lines treated with CXR1002 and Cisplatin. Plots show percentage cell viability of cells treated in combination (squares) compared to Cisplatin alone (triangles).

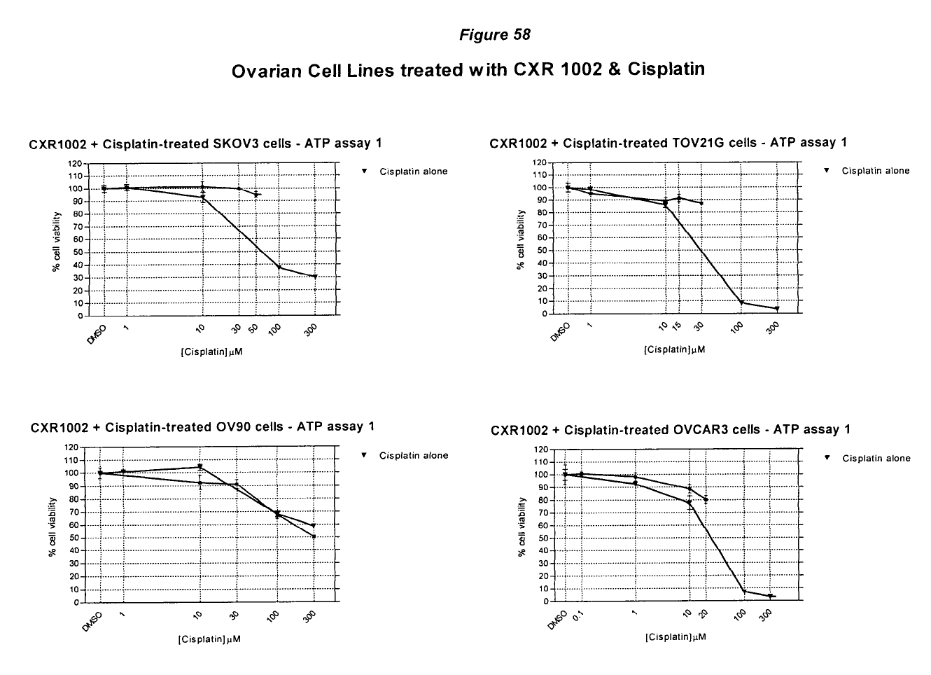

FIG. 58 shows cytotoxicity plots for ovarian cell lines treated with CXR1002 and Cisplatin. Plots show percentage cell viability of cells treated in combination (squares) compared to Cisplatin alone (triangles).

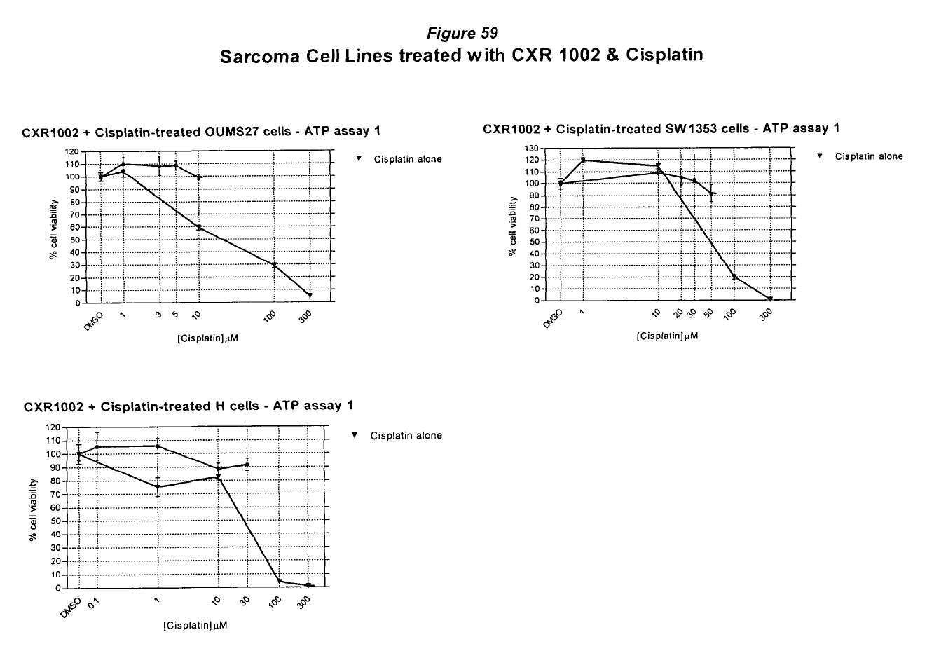

FIG. 59 shows cytotoxicity plots for sarcoma cell lines treated with CXR1002 and Cisplatin. Plots show percentage cell viability of cells treated in combination (squares) compared to Cisplatin alone (triangles).

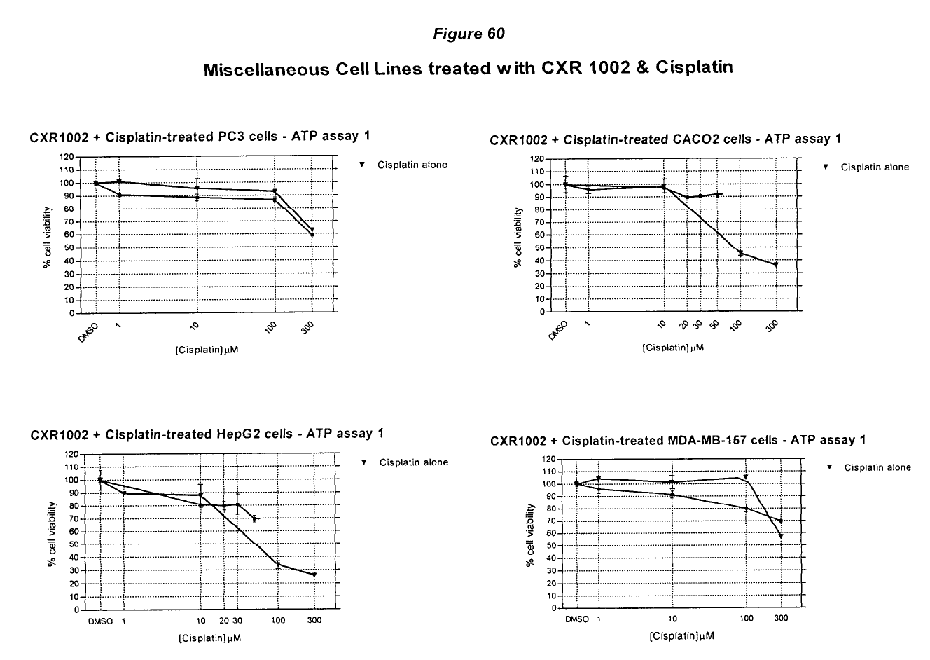

FIG. 60 shows cytotoxicity plots for further cell lines treated with CXR1002 and Cisplatin. Plots show percentage cell viability of cells treated in combination (squares) compared to Cisplatin alone (triangles).

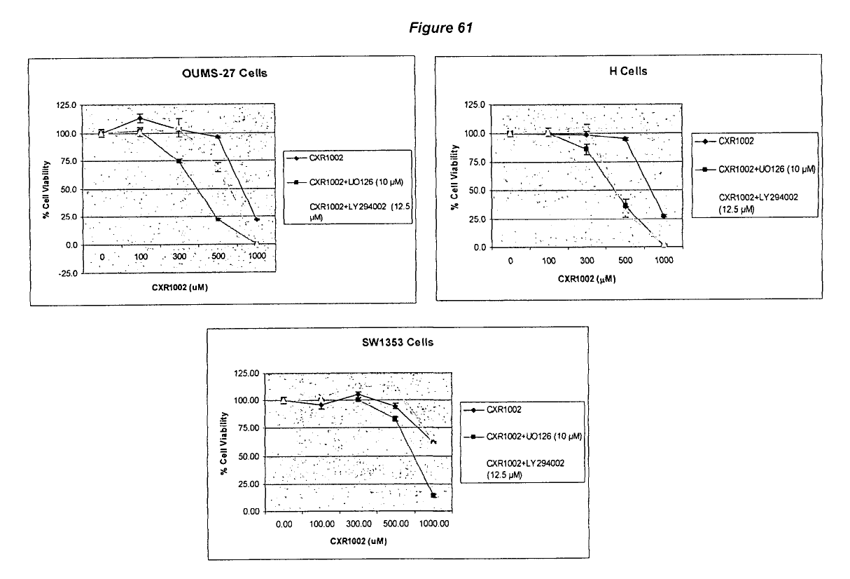

FIG. 61 shows cytotoxicity plots for OMUS-27, H and SW1353 cells treated with CXR1002 alone (diamonds), in combination with UO126 (squares) and in combination with LY294002 (triangles).

FIG. 62 shows cytotoxicity plots for PANC1, BxPC3, HPAFII and Capan2 cells treated with CXR1002 alone (diamonds), in combination with UO126 (squares) and in combination with LY294002 (triangles).

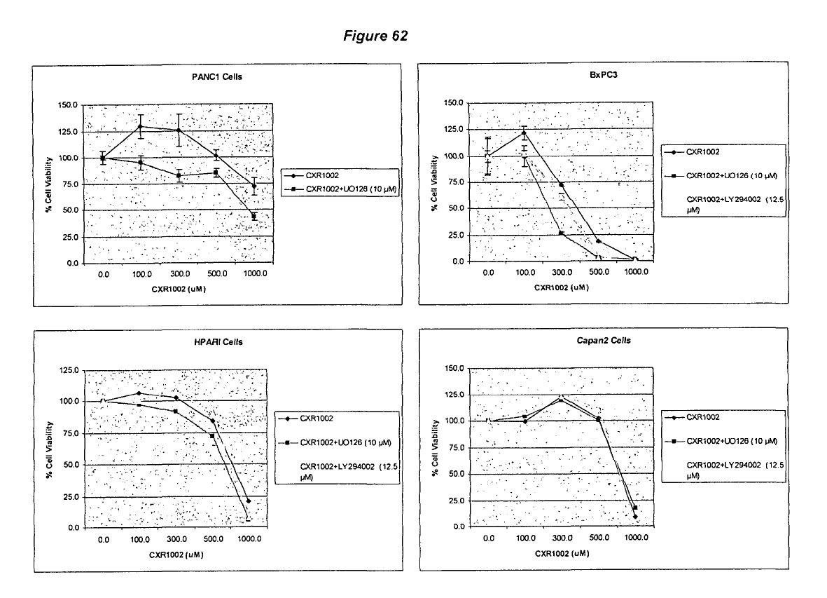

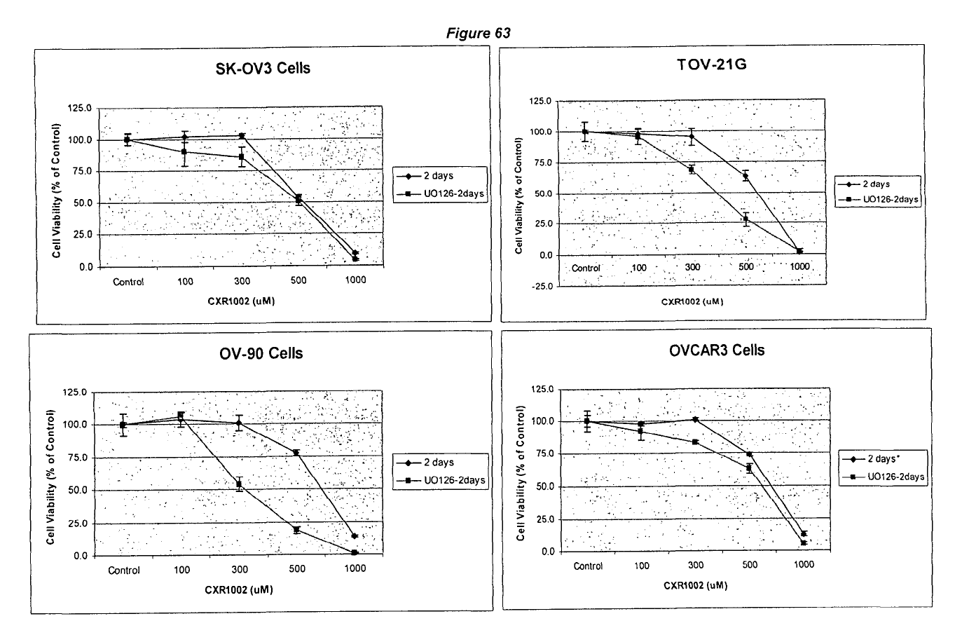

FIG. 63 shows cytotoxicity plots for SK-OV3, TOV-21G, OV-90 and OVCAR3 cells treated with CXR1002 alone (diamonds) or in combination with UO126 (squares).

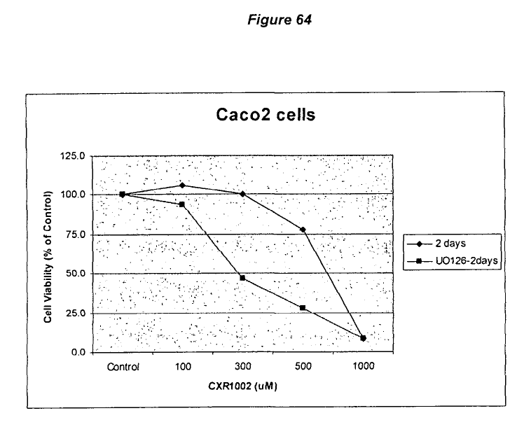

FIG. 64 shows a cytotoxicity plot Caco2 cells treated with CXR1002 alone (diamonds) or in combination with UO126 (squares).

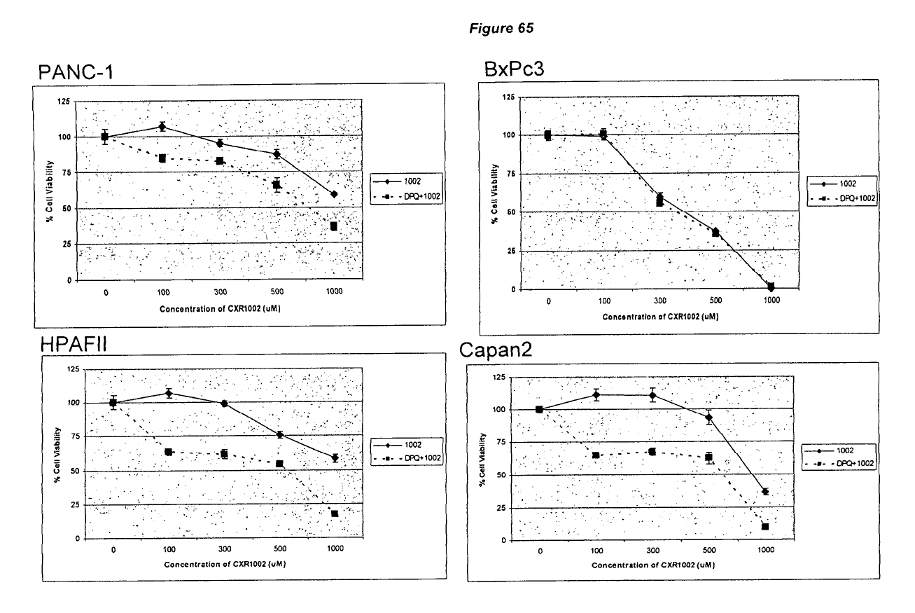

FIG. 65 shows cytotoxicity plots for PANC-1, BxPc3, HPAFII and Capan2 cells treated with CXR1002 alone (diamonds) or in combination with DPQ (squares).

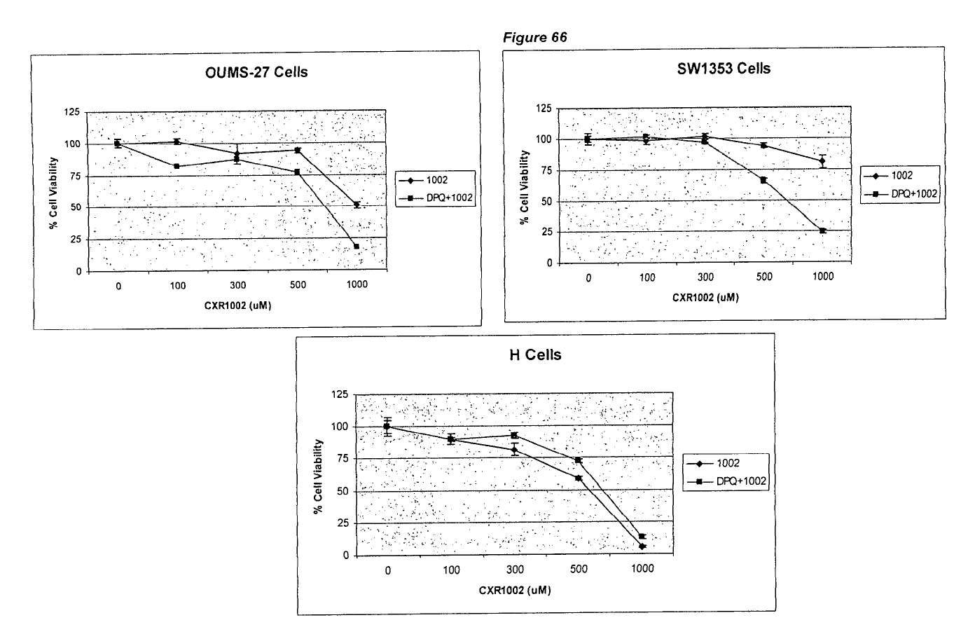

FIG. 66 shows cytotoxicity plots for OUMS-27, SW1353 and H cells treated with CXR1002 alone (diamonds) or in combination with DPQ (squares).

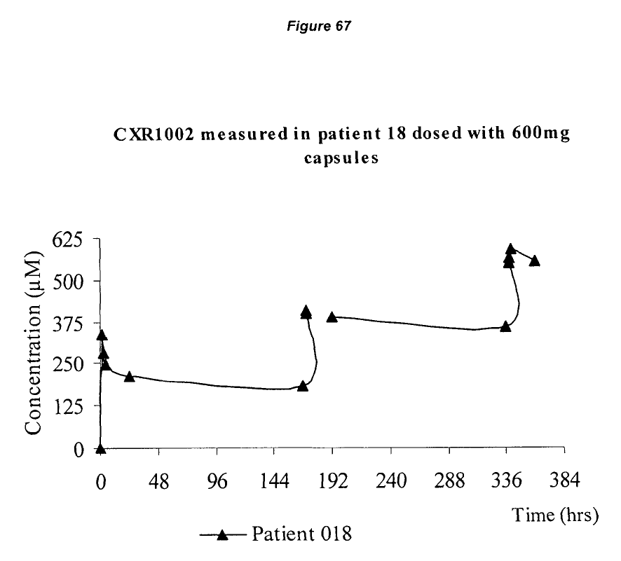

FIG. 67 shows accumulating levels of CXR1002 following a repeat weekly 600 mg dose for patient 18.

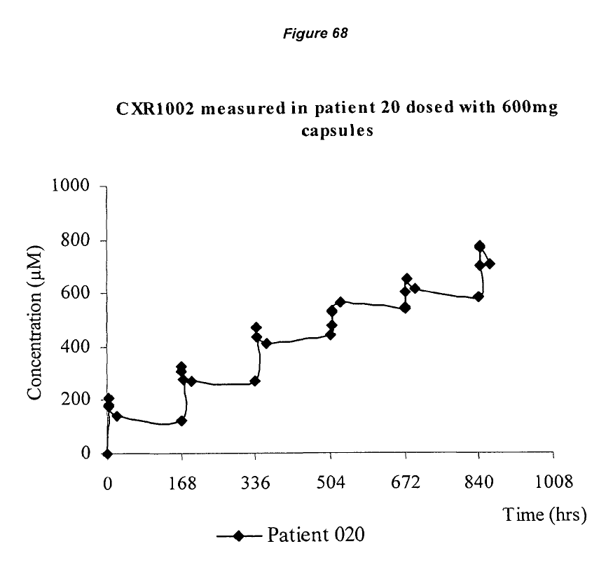

FIG. 68 shows accumulating levels of CXR1002 following a repeat weekly 600 mg dose for patient 20.

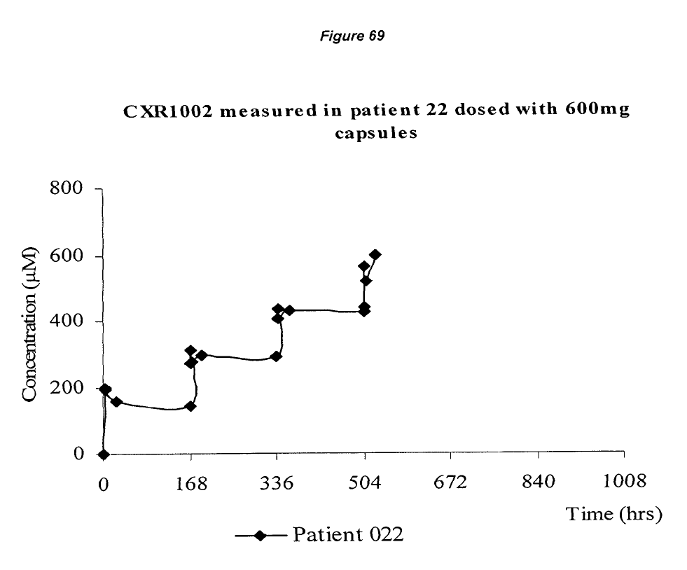

FIG. 69 shows accumulating levels of CXR1002 following a repeat weekly 600 mg does for patient 22.

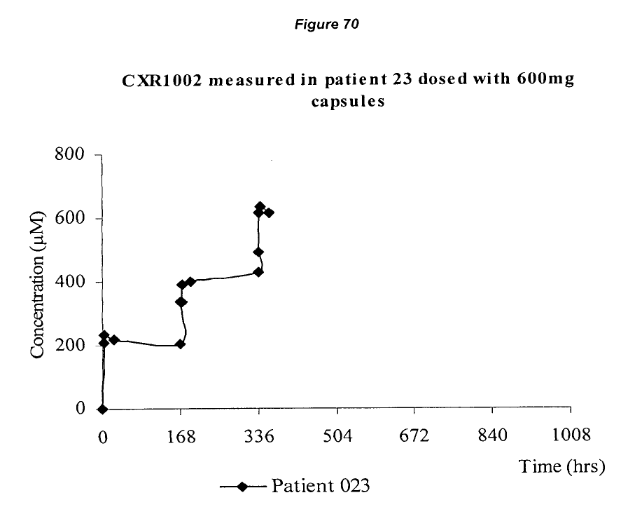

FIG. 70 shows accumulating levels of CXR1002 following a repeat weekly 600 mg dose for patient 23.

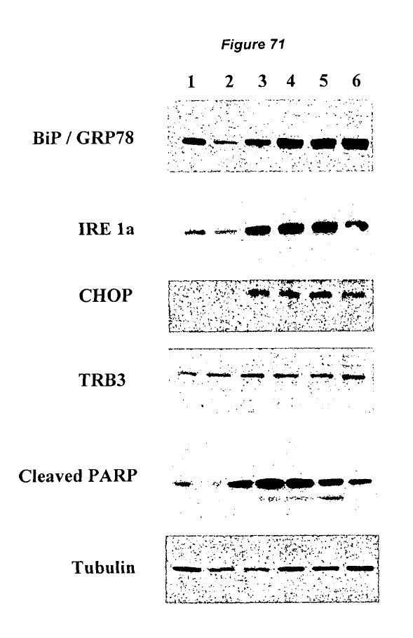

FIG. 71 shows the effect of CXR1002 treatment or induction of expression of ER stress-regulated proteins. Lane designations are given in Example 8.

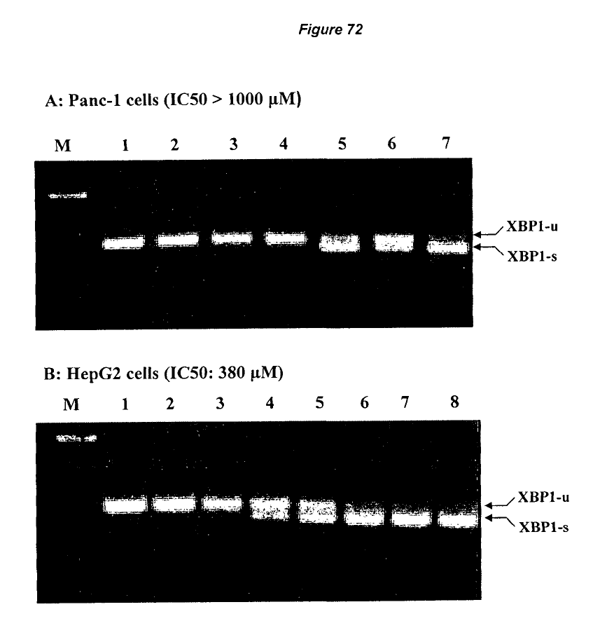

FIG. 72 shows splicing of XBPI mRNA induced in relation to CXR1002 induced ER stress.

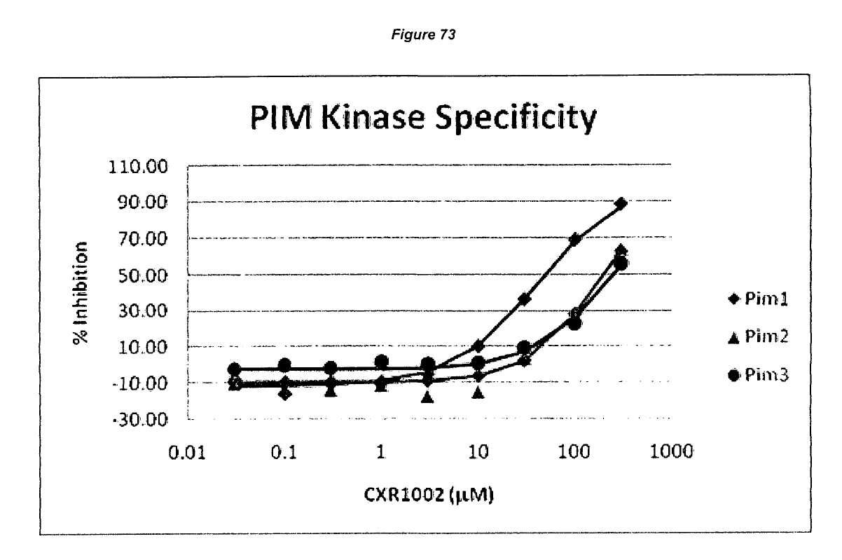

FIG. 73 shows the percentage inhibition of PIM 1, PIM 2 and PIM 3 kinases as a dose response to CXR1002 exposure.

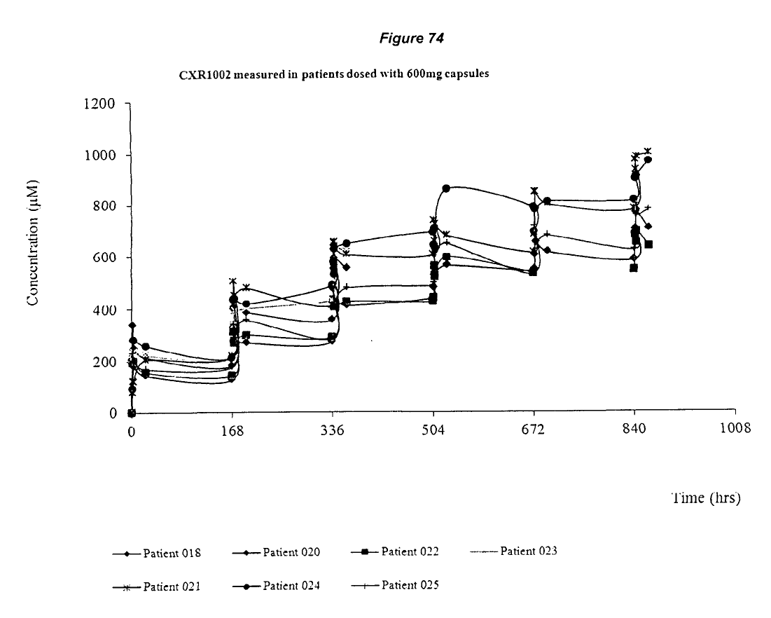

FIG. 74 shows CXR1002 plasma concentrations for a cohort of 6 patients after a repeat weekly 600 mg dose.

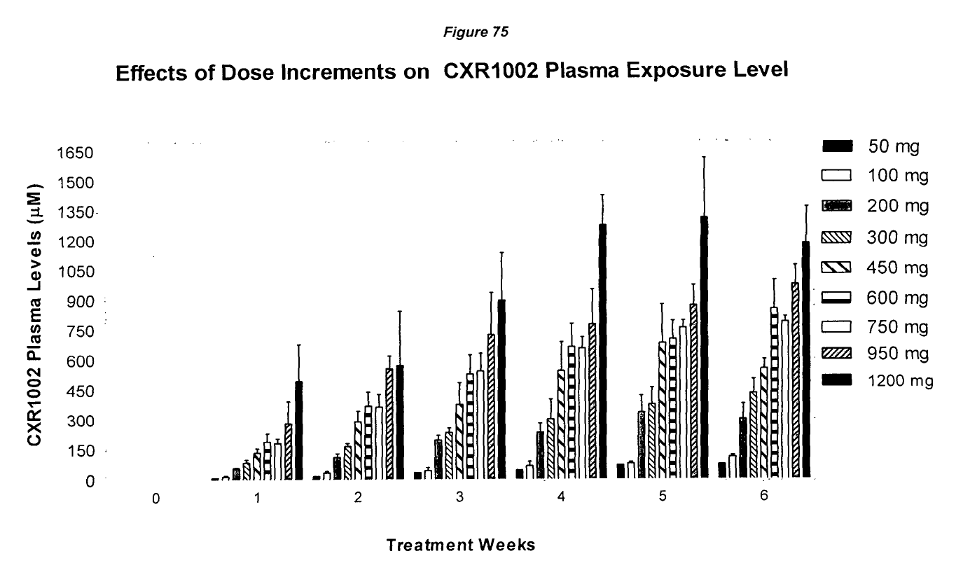

FIG. 75 shows the effects of dose increments on CXR1002 plasma exposure level over 6 weeks.

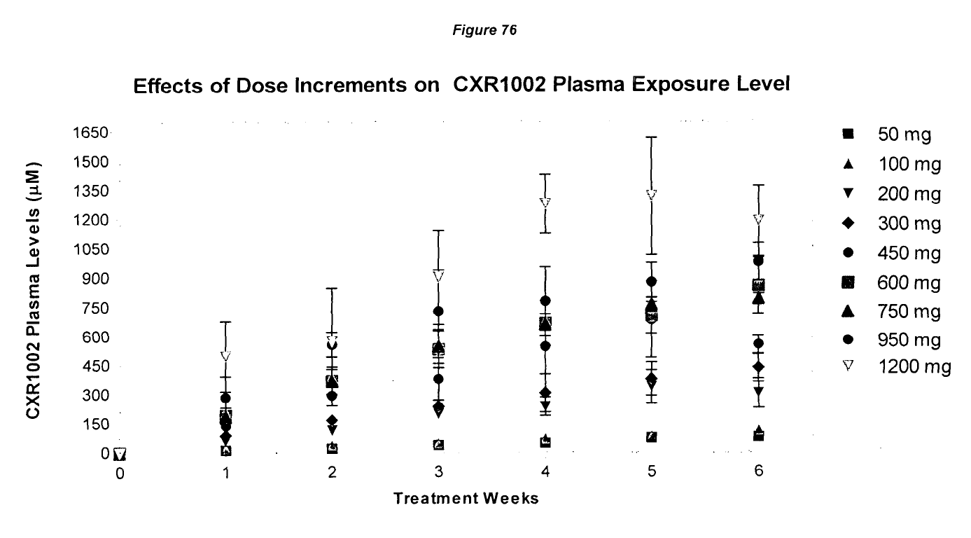

FIG. 76 shows the effects of dose increments on CXR1002 plasma exposure level over 6 weeks. Time points shown refer to pre-dose (TO) and thereafter (weekly) 24 hours post dose.

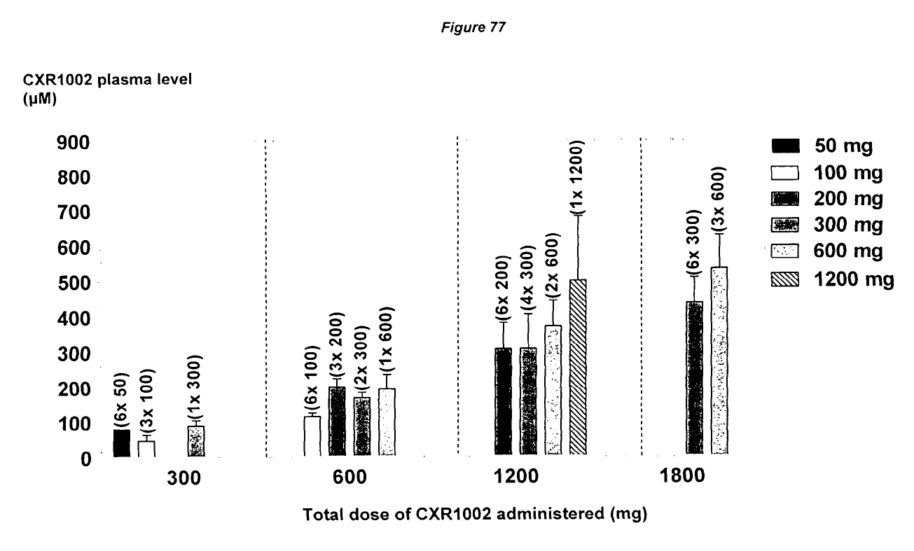

FIG. 77 shows the effect of dose increment on CXR1002 pharmacokinetics.

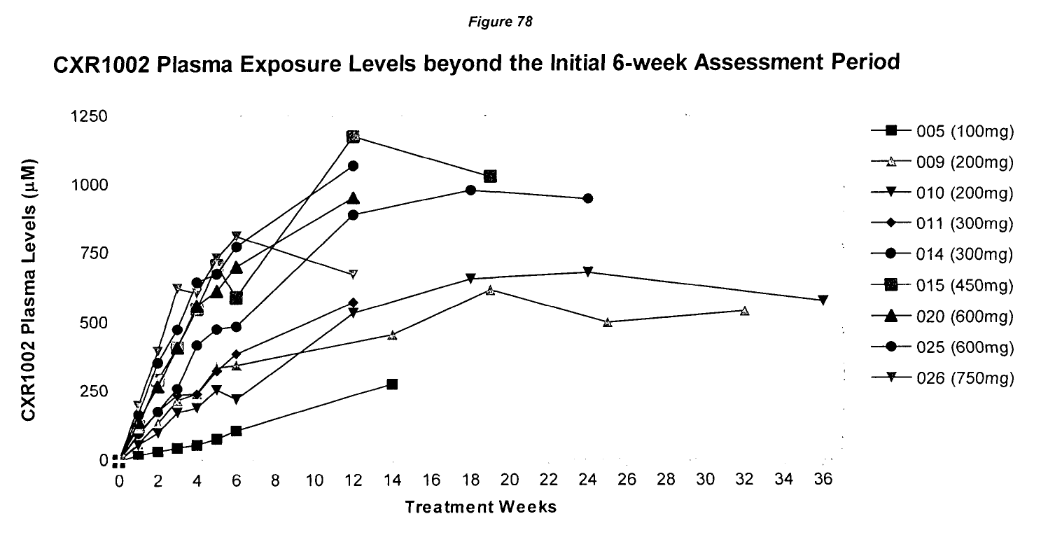

FIG. 78 shows the effect of dose increment on CXR1002 plasma exposure levels beyond the initial 6 week assessment period.

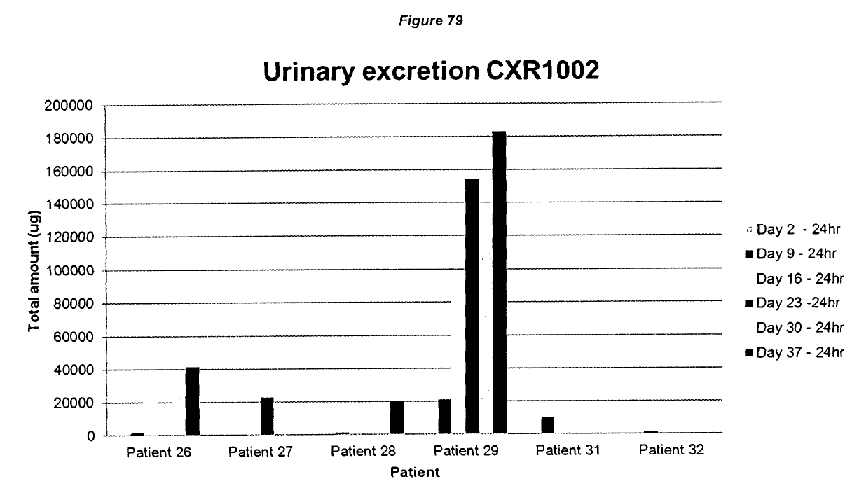

FIG. 79 shows the increase in urinary excretion of CXR1002 with duration of dosing.

FIG. 80 shows that the excretion of CXR1002 is reflected in the pharmacokinetic profile of a patient with high levels of urinary excretion.

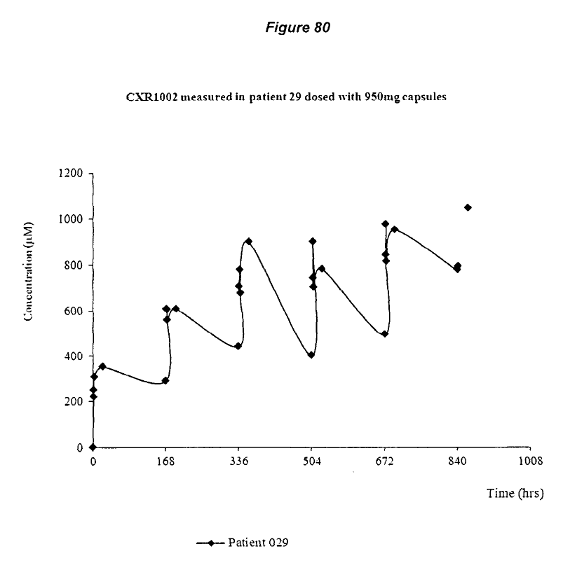

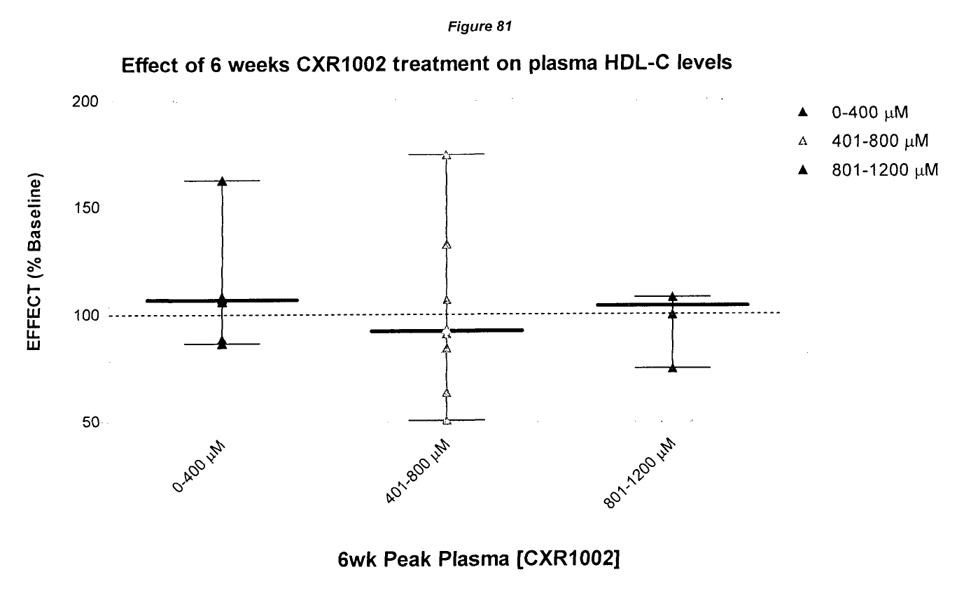

FIG. 81 shows the effect of 6 weeks of CXR1002 treatment on plasma HDL-C levels.

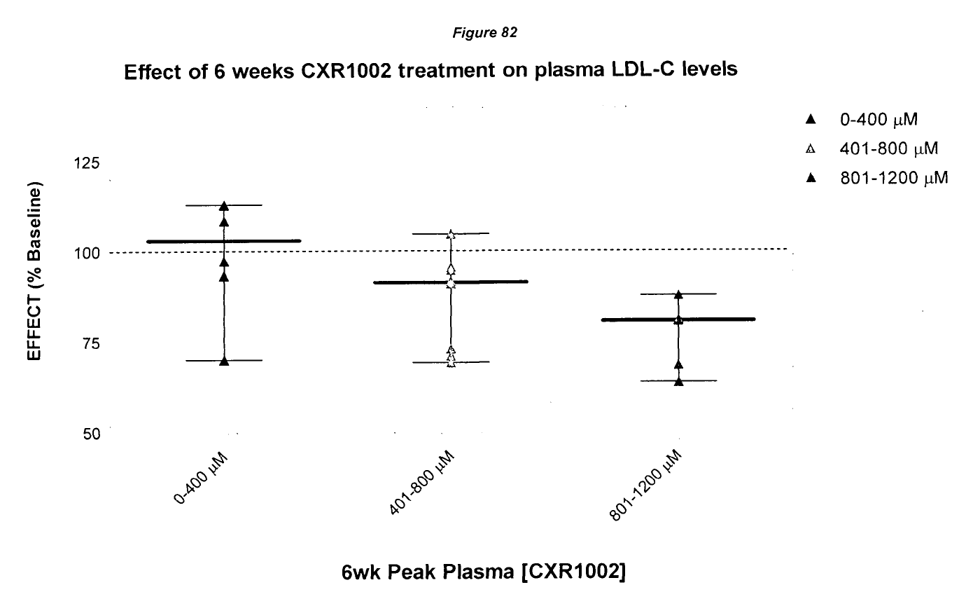

FIG. 82 shows the effect of 6 weeks of CXR1002 treatment on plasma LDL-C levels.

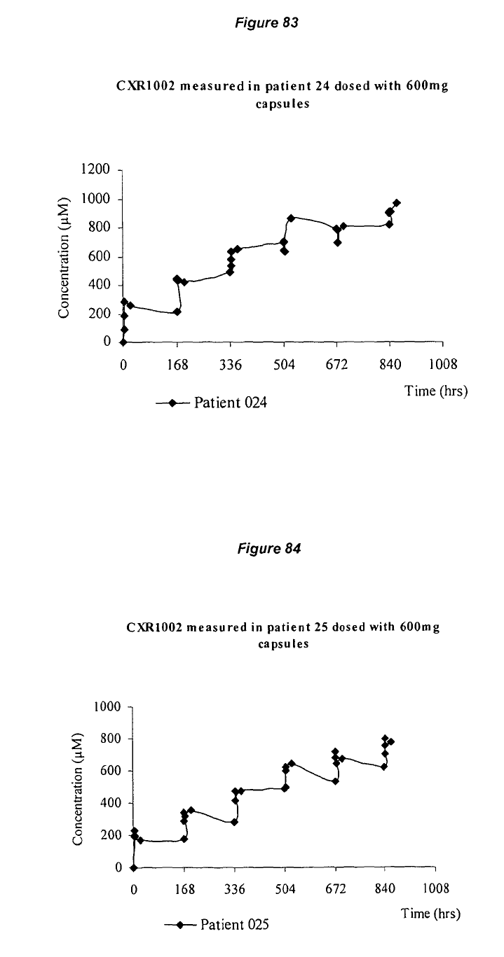

FIG. 83 shows accumulating levels of CXR1002 following a repeat weekly 600 mg dose for 6 weeks in patient 024.

FIG. 84 shows accumulating levels of CXR1002 following a repeat weekly 600 mg dose for 6 weeks in patient 025.

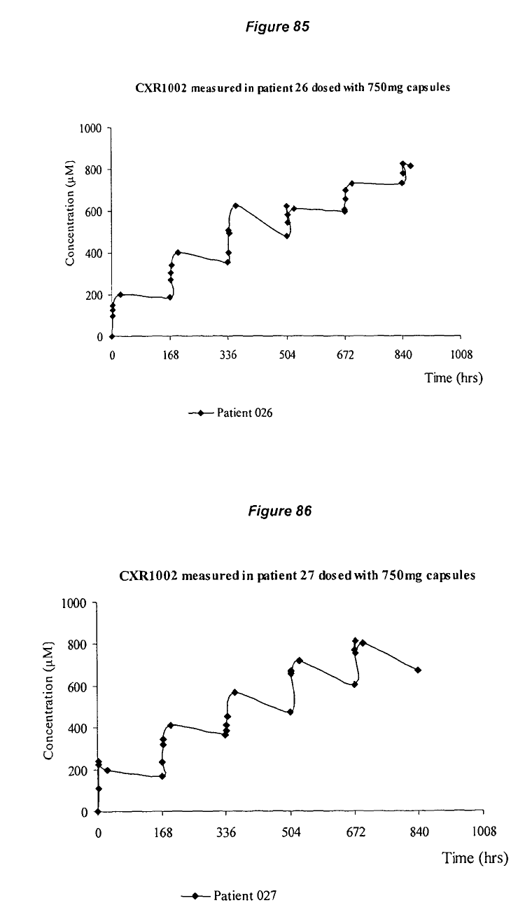

FIG. 85 shows accumulating levels of CXR1002 following a repeat weekly 750 mg dose for 6 weeks in patient 026.

FIG. 86 shows accumulating levels of CXR1002 following a repeat weekly 750 mg dose for 6 weeks in patient 027.

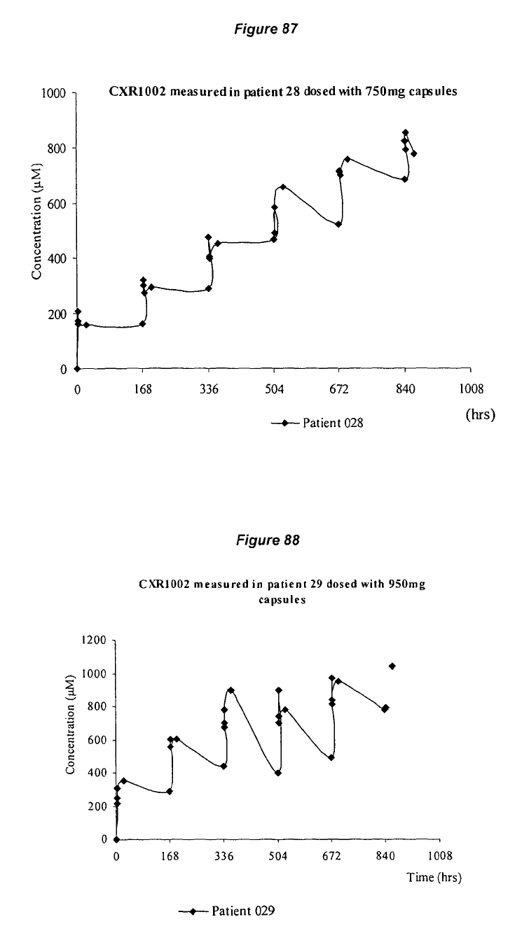

FIG. 87 shows accumulating levels of CXR1002 following a repeat weekly 750 mg dose for 6 weeks in patient 028.

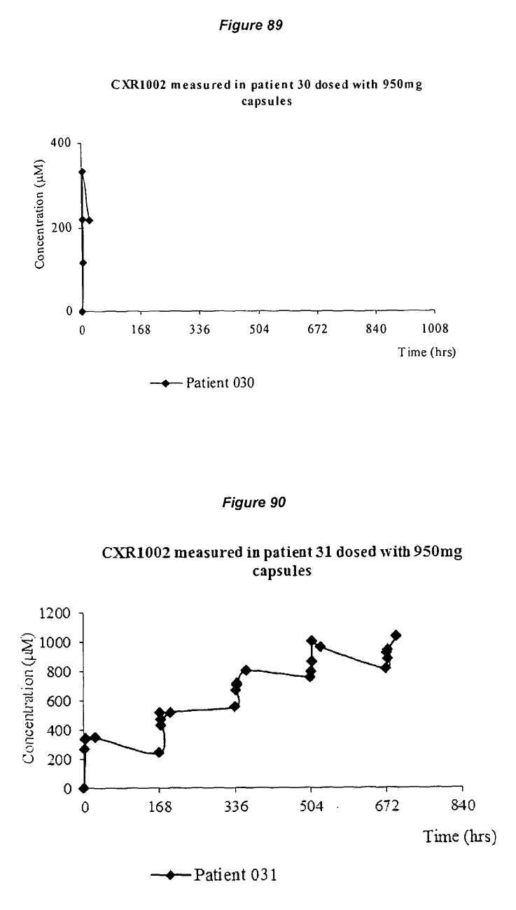

FIG. 88 shows accumulating levels of CXR1002 following a repeat weekly 950 mg dose for 6 weeks in patient 029.

FIG. 89 shows accumulating levels of CXR1002 following a repeat weekly 950 mg dose for 6 weeks in patient 030.

FIG. 90 shows accumulating levels of CXR1002 following a repeat weekly 950 mg dose for 6 weeks in patient 031.

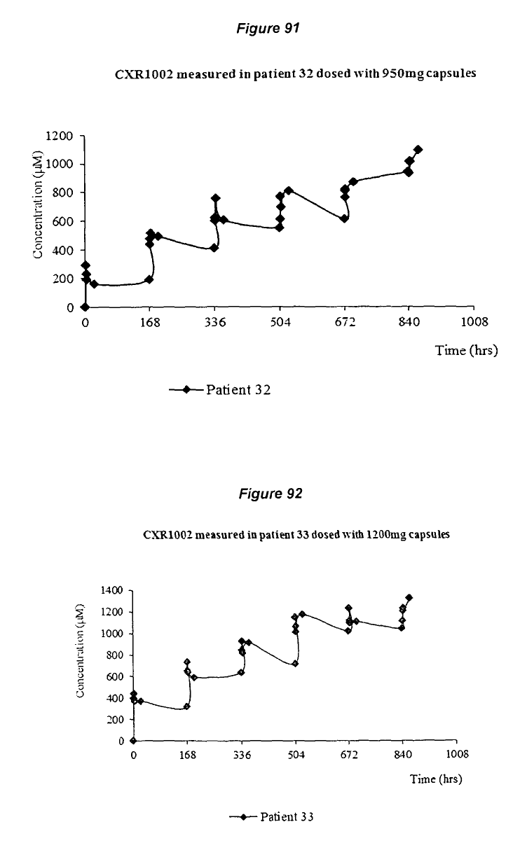

FIG. 91 shows accumulating levels of CXR1002 following a repeat weekly 950 mg dose for 6 weeks in patient 032.

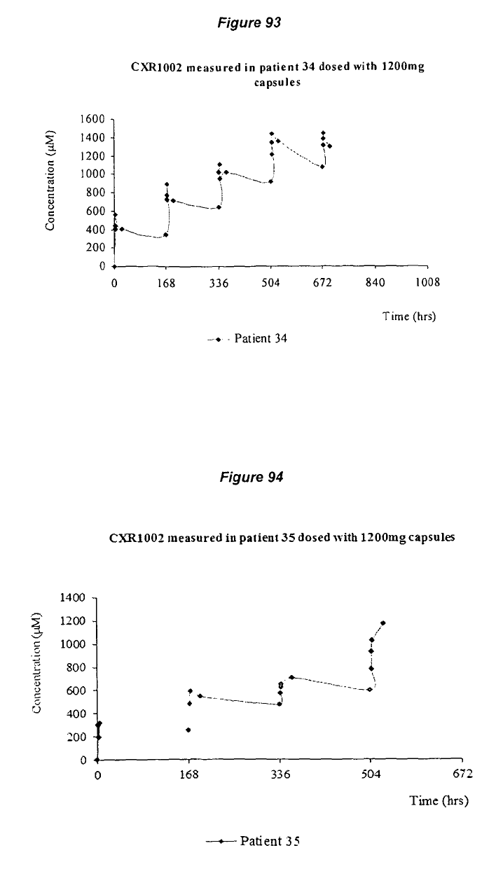

FIG. 93 shows accumulating levels of CXR1002 following a repeat weekly 1200 mg dose for 6 weeks in patient 033.

FIG. 94 shows accumulating levels of CXR1002 following a repeat weekly 1200 mg dose for 6 weeks in patient 034.

FIG. 94 shows accumulating levels of CXR1002 following a repeat weekly 1200 mg dose for 6 weeks in patient 035.

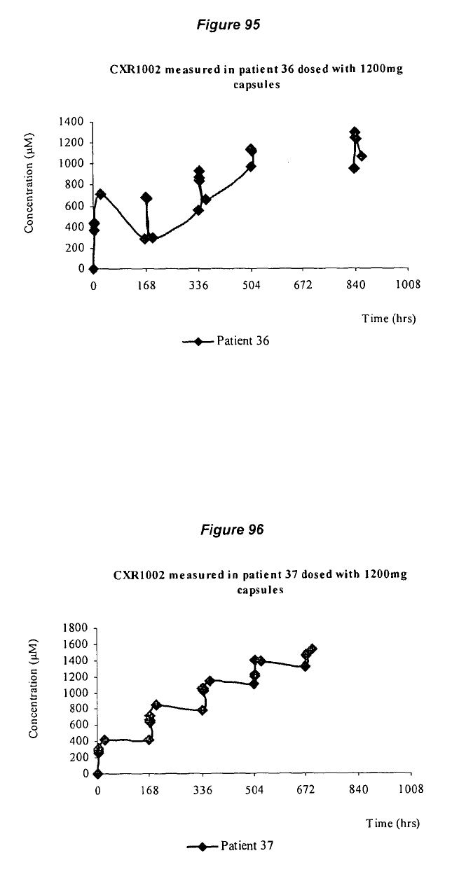

FIG. 95 shows accumulating levels of CXR1002 following a repeat weekly 1200 mg dose for 6 weeks in patient 036.

FIG. 96 shows accumulating levels of CXR1002 following a repeat weekly 1200 mg dose for 6 weeks in patient 037.

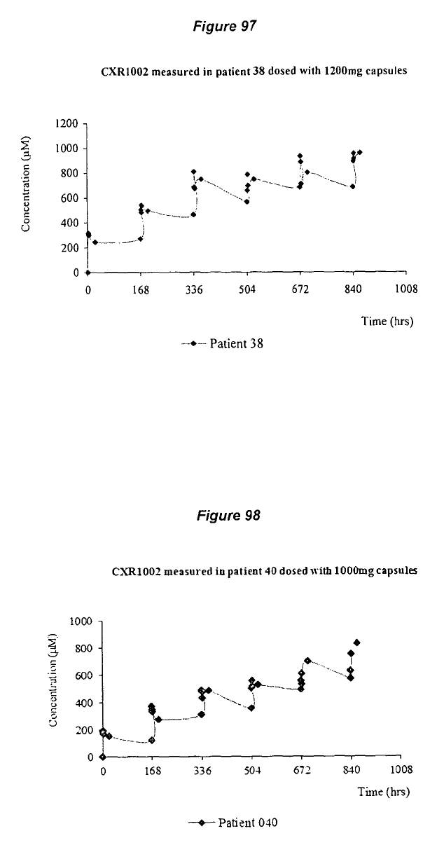

FIG. 97 shows accumulating levels of CXR1002 following a repeat weekly 1200 mg dose for 6 weeks in patient 038.

FIG. 98 shows accumulating levels of CXR1002 following a repeat weekly 1000 mg dose for 6 weeks in patient 040.

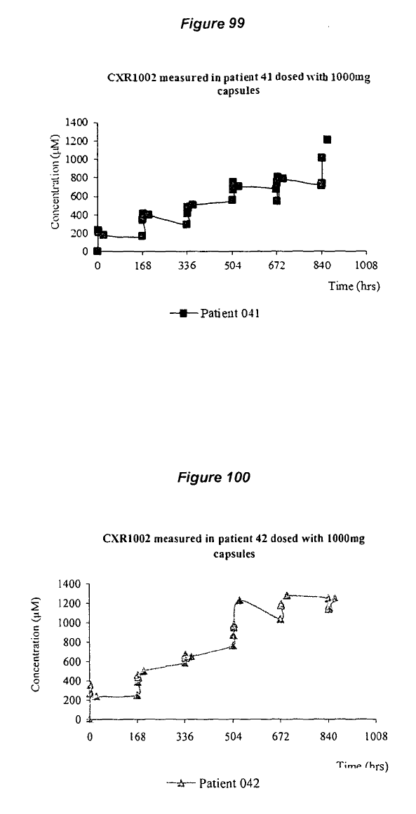

FIG. 99 shows accumulating levels of CXR1002 following a repeat weekly 1000 mg dose for 6 weeks in patient 041.

FIG. 100 shows accumulating levels of CXR1002 following a repeat weekly 1000 mg dose for 6 weeks in patient 042.

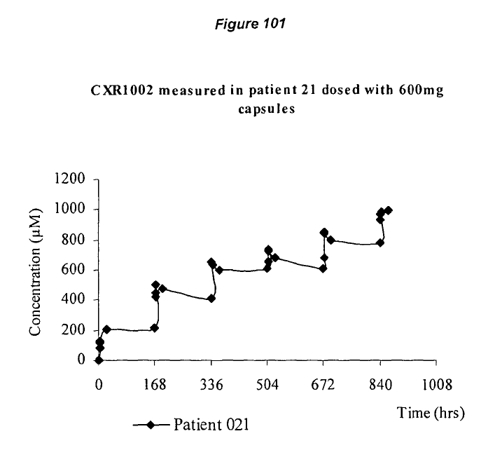

FIG. 101 shows accumulating levels of CXR1002 following a repeat weekly 600 mg dose for 6 weeks in patient 021.

### PREFERRED EMBODIMENTS

### Example 1

### Induction of Peroxisome Proliferation

The earliest recognised characteristic of PPARα agonists was their ability to induce peroxisome proliferation in hepatocytes. The PPARα response is reflected in the increased transcription of mitochondrial and peroxisomal lipid metabolism, sterol, and bile acid biosynthesis and retinol metabolism genes (Andersen (2008)). Administration of APFO to rats led to hepatic peroxisome proliferation as measured by the induction of the peroxisomal marker activity cyanide-insensitive palmitoyl CoA oxidation (unpublished data).

Peroxisome proliferation occurs as a result of the interaction of a chemical with PPARα. This leads to an increase in the synthesis of peroxisomal and lipid-metabolising enzymes and, consequently, an increase in size and number of peroxisomes. Cyanide-insensitive palmitoyl CoA oxidation is an accepted marker of peroxisome proliferation, and was used to highlight PPARα activation in vitro and in vivo.

In vivo, APFO exhibits aspects of pharmacology typical of both PPARα and γ agonism. Male Sprague Dawley rats (n=6) were administered APFO (300 ppm) in powdered diet daily while control animals received powdered diet only. Rats were sacrificed at 7, 14, 28 and 84 days. Blood from each study animal was taken by cardiac puncture into lithium/heparin-coated tubes for separation of plasma. Plasma was analysed for glucose, triglycerides, cholesterol, AST and ALT (unpublished data).

Administration of APFO resulted in decreases in the plasma concentrations of triglycerides (PPARα-mediated) and glucose (PPARγ); plasma cholesterol levels were also reduced at all time points (Table 1). No adverse clinical observations were noted even after one year of continuous dietary dosing, although at early time points (1-2 weeks) slight elevations in plasma aspartate and alanine aminotransferase (AST and ALT) levels were observed. At this dietary dose level (300 ppm), plasma concentrations of APFO were 157.00±77.80 μM at week 2 and 256.96±38.93 μM at week 4.

TABLE 1
Effects of APFO on nutritional homeostasis in the
rat. Data shown are mean ± standard deviation.
Nutritional parameters

| | Glucose | | Triglycerides | | Cholesterol | |
|---|---|---|---|---|---|---|
| Week | Control | APFO | Control | APFO | Control | APFO |
| 1 | 19.00 ± 2.28 | 14.31 ± 1.88*** | 1.42 ± 0.40 | 0.41 ± 0.12*** | 2.18 ± 0.22 | 1.27 ± 0.41*** |
| 2 | 24.53 ± 5.53 | 16.98 ± 3.21** | 1.58 ± 0.31 | 0.62 ± 0.13*** | 1.96 ± 0.34 | 1.55 ± 0.27** |
| 4 | 25.19 ± 6.92 | 15.34 ± 2.64** | 1.80 ± 0.79 | 0.57 ± 0.13*** | 2.30 ± 0.22 | 1.70 ± 0.29*** |
| 12 | 17.12 ± 2.36 | 13.12 ± 1.23*** | 1.69 ± 0.55 | 0.63 ± 0.15*** | 2.17 ± 0.26 | 1.75 ± 0.33* |

Indicators of liver toxicity

| | AST | | ALT | |
|---|---|---|---|---|
| Days | Control | APFO | Control | APFO |
| 1 | 107.80 ± 10.12 | 128.88 ± 6.21*** | 98.60 ± 9.01 | 101.90 ± 12.19 |
| 2 | 100.63 ± 4.63 | 120.00 ± 16.47** | 81.60 ± 8.10 | 119.45 ± 19.27*** |
| 4 | 97.13 ± 13.56 | 101.80 ± 19.17 | 87.93 ± 11.54 | 98.99 ± 24.44 |
| 12 | 79.50 ± 9.86 | 92.63 ± 12.19* | 71.67 ± 6.8 | 91.89 ± 14.88** |

Statistical significance:
*p ≦ 0.05;
**p ≦ 0.01;
***p ≦ 0.001

Interaction of CXR1002 with PPARs

Activation of PPARs is a transcriptional signature for PFOA in rats and mice, as well as common carp and zebrafish (Andersen (2008)). The effects of APFO and CXR1002 on the three PPAR isoforms in Cos-1 cells using a GAL4 binding assay and a transactivation assay using full length PPAR reporter gene constructs have been conducted using truncated PPAR constructs. The transactivation assay was performed in both agonist and antagonist mode (unpublished data). In antagonist mode for PPAR the finding from earlier assays suggesting reduced reporter expression, was confirmed by observation of direct antagonism activity for CXR1002. These findings are in keeping with those reported in independent studies by Vanden Heuvel et al., (2006) and Takacs & Abbott (2007), and are summarized together in Table 2.

### Effects of CXR1002 on Other Nuclear Receptors

The effects of CXR1002 are not limited to PPARs. The non-selective pan-activation of numerous nuclear receptors is apparent not only by the transcriptional activation of many genes in PPARα-null mice (Rosen (2008)), but also by the scope of metabolic and regenerative pathways elicited by CXR1002 exposure. In particular, constitutive androstane receptor (CAR) and pregnenolone X receptor (PXR) are activated (Ren (2009)), although this appears to be on a species-specific basis. Further studies are needed, particularly on the human genes, to determine the significance of this in humans. Neither liver X receptor β (LXRβ) nor the common heterodimerization partner retinoid X receptor α (RXRα) are activated by PFOA (14).

TABLE 2
PPAR isoform agonism and antagonism reported using various assay
systems.

| | Dose CXR1002 or | | | |
|---|---|---|---|---|
| | PFOA (μM) | | | |
| Assay | 30 | 100 | 300 | Reference |

PPARα

| | | | | |
|---|---|---|---|---|
| Human PPARα ligand binding | − | + | + | (12) |
| Human PPARα transactivation - | − | − | ++ | (12) |
| full length in Cos-1 cells | | | | |
| Human PPARα transctivation - | − | + | ND | (13) |
| truncated in HEK293 T cells | | | | |
| (agonist mode) | | | | |
| Human PPARα transctivation - | − | − | ND | (13) |
| truncated in HEK293 T cells | | | | |
| (antagonist mode using 10 μM | | | | |
| ciprofibrate) | | | | |
| Human PPARα transactivation | + | ND | ND | (15) |
| in Cos-1 cells | | | | |
| Human PPARα transactivation | ND | ++ | ND | (14) |
| in 3T3-L1 cells | | | | |

PPARγ

| | | | | |
|---|---|---|---|---|
| Human PPARγ ligand binding | − | − | ++ | (12) |
| Human PPARγ transactivation - | − | − | + | (12) |
| full length in Cos-1 cells | | | | |
| Human PPARγ transctivation - | − | − | − | (13) |
| truncated in HEK293 T cells | | | | |
| (agonist mode) | | | | |
| Human PPARγ transctivation - | − | − | + | (13) |
| truncated in HEK293 T cells | | | | |
| (antagonist mode using 1 μM | | | | |
| rosiglitazone) | | | | |
| Human PPARγ transactivation | − | − | ND | (15) |
| in Cos-1 cells | | | | |
| Human PPARγ transactivation | ND | − | ND | (14) |
| in 3T3-L1 cells | | | | |

PPARδ

| | | | | |
|---|---|---|---|---|
| Human PPARδ ligand binding | − | − | − | (12) |
| Human PPARδ transactivation - | − | − | − | (12) |
| full length in Cos-1 cells | | | | |
| Human PPARδ transctivation - | − | − | ND | (13) |
| truncated in HEK293 T cells | | | | |
| (agonist mode) | | | | |
| Human PPARδ transctivation - | + | ++ | ND | (13) |
| truncated in HEK293 T cells | | | | |
| (antagonist mode using 100 μM | | | | |
| bezafibrate) | | | | |
| Human PPARδ transactivation | − | ND | ND | (15) |
| in Cos-1 cells | | | | |
| Human PPARδ transactivation | ND | − | ND | (14) |
| in 3T3-L1 cells | | | | |

ND = not done

### Example 2

### CXR1002 Induces ER Stress in Human Tumour Cells

To investigate the anti-tumour effects of CXR1002 in a non-biased manner, transcription profiling analysis was performed using the human pancreatic carcinoma cell line PANC-1 cultured in vitro. Gene expression changes observed in the normal pancreas are different from those in the liver, and suggest possible effects on gluconeogenesis and glutamine metabolism (Anderson (2008)). PANC-1 cells were treated with CXR1002 for 24 hrs at a concentration that has been found to cause 15% inhibition of cell growth (IC₁₅) and RNA was subsequently extracted. Analysis of the transcription profiles was made using pathways analysis in the Ingenuity system (unpublished data).

A list of 4996 genes was generated that showed changes in the treated samples compared to the untreated samples. Representation analysis of the in vitro 4996 signature list identified a number of pathways that were over-represented. In particular, genes in the endoplasmic reticulum (ER) stress pathway were over-represented in the signature list, FIG. 1; Table 3. This included the ATF family of transcription factors (ATF3, ATF4 and ATF6) which are responsible for inducing ER stress and the unfolded protein response (UPR) (Szegezdi (2006)). ATF3 (induced-3 fold) was identified as a key transcription factor and pivotal component of the ER stress pathway.

The endoplasmic reticulum (ER) serves two major functions in the cell. It facilitates the proper folding of newly synthesised proteins destined for secretion and it provides the cell with a calcium reservoir. ER stress occurs in various physiological and pathological conditions where the capacity of the ER to fold proteins becomes saturated. Examples of these situations include calcium flux, glucose starvation, hypoxia or defective protein secretion, modification or degradation.

TABLE 3
Gene changes connected to ER stress in CXR1002-treated PANC-1 cells, as determined using
Ingenuity Pathways Analysis software.

| | | | Gene | Fold | p- |
|---|---|---|---|---|---|
| Symbol | Synonyms | Entrez Gene Name | Function | change | Value* |
| ATF4 | C/ATF, CREB- | Activating | transcription | 1.818 | 2.47E−13 |
| | 2, MGC96460, | transcription factor 4 | regulator | | |
| | TAXREB67, | (tax-responsive | | | |
| | TXREB | enhancer element | | | |
| | | B67) | | | |
| ATF6 | ATF6 ALPHA, | Activating | transcription | 1.763 | 2.62E−10 |
| | ATF6A, | transcription factor 6 | regulator | | |
| | ESTM49 | | | | |
| CASP9 | CASPASE-9, | Caspase 9, | peptidase | 1.291 | 2.40E−04 |
| | CASPASE-9c | apoptosis-related | | | |
| | | cysteine peptidase | | | |
| EIF2AK3 | PERK, WRS | Eukaryotic | kinase | −1.266 | 4.62E−03 |
| | | translation initiation | | | |
| | | factor 2-alpha kinase 3 | | | |
| HSPA5 | GRP78, HEAT | Heat shock 70 kDa | other | 2.414 | 7.23E−35 |
| | SHOCK 70 KDA | protein 5 (glucose- | | | |
| | PROTEIN5 | regulated protein, 78 kDa) | | | |
| MAPK8 | C-JUN N- | Mitogen-activated | kinase | 1.097 | 3.14E−03 |
| | TERMINAL | protein kinase 8 | | | |
| | KINASE1, JNK, | | | | |
| | JNK1 | | | | |
| MBTPS1 | PCSK8 | Membrane-bound | peptidase | −1.170 | 7.60E−03 |
| | | transcription factor | | | |
| | | peptidase, site 1 | | | |
| TAOK3 | JIK, MAP3K18 | TAO kinase 3 | kinase | 1.274 | 3.70E−04 |
| XBP1 | HTF, Sxbp-1, | X-box binding protein 1 | transcription | 1.689 | 3.41E−10 |
| | TREB-5, XBP2 | | regulator | | |

*The p value calculated by Fishers test represents the probability that the association between genes in the signature list and the cannonical pathway (in this case ER stress) occurred by chance alone.

Cells respond to the accumulation of unfolded proteins in the ER by a rescue process called the unfolded protein response (UPR). However, if the unfolded protein accumulation is persistent and the stress cannot be relieved, UPR signalling switches from prosurvival to proapoptotic (Kim (2006)), (Szegezdi (2006)), usually involving processing of caspases (Chang (2006)). Consistent with this hypothesis, CXR1002-treated PANC-1 cells show reduced proliferation and cleavage of the caspase substrate poly-ADP ribose polymerase (PARP) (FIG. 2) in PANC-1 cells.

Disruption of the UPR is particularly significant in certain tissues or organs, particularly those dedicated to extracellular protein synthesis e.g. glandular tissues such as the pancreas and thyroid. The pancreatic β-cell is particularly dependent on efficient UPR signalling due to the constantly varying demands for insulin synthesis (Marciniak (2006)).

Chemical toxicants such as tunicamycin and thapsigargin cause an accumulation of unfolded protein aggregates in the ER lumen (Schroder (2008)), (Harding (2002)), (Zhang (2008)). Whilst it is fair to say that many chemicals, drugs and toxicants induce ER stress, not all do. Microarray data from a previous unpublished study requires further analysis, but superficially at least seems to indicate that the ER stress effect may be specific to the pancreatic cancer cell line PANC-1 and not a feature of normal pancreas tissue since the ER stress response is not seen in normal pancreas treated with APFO (28 day study in rat). The phthalate DEHP and the PPARα agonist WY14,643 were also studied. No evidence of ER stress response was detected with either of these compounds.

In studies of primary rat hepatocytes, PFOA concentrations of 30 μM and above caused increased expression of DNA damage-inducible transcript 3 (DDIT3/CHOP/GADD153), suggesting ER stress (Bjork (2009)).

ER stress can be caused by the induction of oxidative enzymes and the CXR1002 PANC-1 microarray signatures showed some mRNA level induction of enzymes involved in redox homeostasis. Altered genes included glutamate-cysteine ligase modifier subunit (GCLM), glutamate-cysteine ligase catalytic subunit (GCLC), heme oxygenase (HO-1), glutathione reductase (GSR) and thioredoxin reductase (TRXR1) which are reflected by the over-representation of genes in the NRF2 signalling pathway. This is an indication that the PANG-1 cells are undergoing an oxidative stress response. The mechanism of this is unclear, however the induction of DNA damage response genes such as Growth arrest and DNA damage alpha (GADD45α), DDIT3, p21 and p53 suggest that oxidative stress may result in DNA damage. However, in follow-up experiments CXR1002 did not activate transcription of p21^{WAF1}, when examined using β-human chorionic gonadotrophin (hCG) excretion from reporter cell line A2780/p21^{WAF1} exposed to CXR1002 for 24 hrs (unpublished data).

### Discussion of Mechanism of Action/Target

The above data demonstrate that CXR1002 activates both PPARα and PPARγ at similar concentrations, potentially conferring the benefits of both receptors, including growth inhibition, induction of apoptosis and induction of terminal differentiation. Furthermore, CXR1002 may inhibit PPARδ. Given that PPARδ is able to oppose the effects of PPARα and PPARγ (Vosper (2001)) via repression of transcription mediated by competition for DNA binding (Shi (2002)), there may be a benefit to PPAR α/γ agonist which is inhibitory or neutral at the PPARδ receptor. CXR1002 may have effects on other nuclear receptors, such as CAR and PXR.

Induction of ER stress in tumour cells is a mechanistically important mode of action for a variety of anti-cancer drugs including bortezomib (Velcade) (Healy (2009)). It has also been shown to occur in mechanistic studies of PPAR agonists, such as the dual agonist thiazolidinedione TZD18 (Zang (2009)) and PPARγ ligands such as prostaglandin J2 (Weber 2004)), (Chamber (2007)). A direct correlation between ER stress and PPAR effect remains to be determined for CXR1002.

Overloading the UPR to induce cell death is a possible anticancer strategy (Healy (2009)). Recently, the UPR has been linked to hepatic lipid metabolism (Lee (2009)), and the finding that the transcription factor XBP1, best known as a key regulator of the UPR, is required for de novo fatty acid synthesis in the liver suggests this gene or gene pathway to be a key link (Lee (2008)).

### Example 3

### In Vitro Cytotoxicity of APFO and CXR1002

The Sulphorhodamine B (SRB) assay was used to determine the in vitro cytotoxicity of APFO (CXR1001) and CXR1002 towards a panel of human tumour-derived cell lines in a 48 hr assay. The SRB assay was performed according to the method specified by the NIC/NIH. The results for ten cell lines using the SRB assay are summarised in Table 4. The lowest IC₅₀ values (˜160 μM) were seen with HepG2 cells and the highest (˜740 μM) were seen with CaCo-2 cells. In every case the cytotoxic effects of APFO and CXR1002 were similar. In subsequent experiments with CXR1002 an ATP cytotoxicity assay was used on a panel of 18 tumour cell lines. The effects of CXR1002 were assessed after 48 hr treatment. Assay replicates were independent in time and up to 4 replicates were performed per cell line. In this study, some cell lines were resistant to CXR1002, or produced dose response curves which did not allow for IC₅₀ determination. A 48 hr assay may not produce optimal cytotoxicity; recent data shows that a 7 day endpoint gives lower cytotoxicity IC₅₀ values (data not shown).

TABLE 4
In vitro cytotoxicity of APFO and CXR1002 using the SRB assay (48 hrs).

| | | IC₅₀ Values (μM) | |
|---|---|---|---|
| Cell | | After 48 Hrs Exposure | |
| Line | Tissue Type | APFO (CXR1001) | CXR1002 |
| HT-29 | Colon Adenocarcinoma | 283.3 ± 5.8 | 341.7 ± 10.6 |
| CaCo-2 | Colon Adenocarcinoma | 736.7 ± 244.8 | 708.3 ± 159.1 |
| MCF-7 | Mammary | 388.3 ± 227.3 | 275.7 ± 33.2 |
| | Adenocarcinoma | | |
| MDA- | Mammary Carcinoma | 511.7 ± 194.5 | 463.3 ± 167.7 |
| MB-157 | | | |
| HepG2 | Hepatoblastoma | 167.3 ± 97.1 | 161.7 ± 31.8 |
| Hep3B | Hepatocarcinoma | 273.3 ± 80.2 | 215.0 ± 28.3 |
| PC3 | Prostate | 316.7 ± 59.2 | 271.7 ± 40.7 |
| | Adenocarcinoma | | |
| A549 | Non-Small Cell Lung | 223.3 ± 35.4 | 230.0 ± 21.8 |
| | Carcinoma | | |
| A2780 | Ovarian Carcinoma | 260.0 ± 28.3 | 237.7 ± 7.5 |
| A375 | Malignant Melanoma | 230.0 ± 10.0 | 221.7 ± 21.2 |

TABLE 5
In vitro cytotoxicity of CXR1002 using the ATP depletion cytotoxicity assay (48 hrs).

| Cell Line | Tissue type | IC₅₀ 1 | IC₅₀ 2 | IC₅₀ 3 | IC₅₀ 4 | Mean | SD |
|---|---|---|---|---|---|---|---|
| H | Chondrosarcoma | 550 | 555 | 800 | | 635.00 | 142.92 |
| OUMS-27 | Chondrosarcoma | 1000 | 1000 | 800 | 800* | 900.00 | 115.47 |
| SW1353 | Chondrosarcoma | >1000 | >1000 | >1000 | >1000* | >1000 | |
| PANC-1 | Pancreatic | >1000 | >1000 | >1000 | 880** | >1000 | |
| | Epithelioid | | | | | | |
| | carcinoma | | | | | | |
| BxPc3 | Pancreatic | 370 | 350 | 400 | | 373.33 | 25.17 |
| | Adenocarcinoma | | | | | | |
| HPAFII | Pancreatic | >1000 | >1000 | 750 | | >1000 | |
| | Adenocarcinoma | | | | | | |
| Capan2 | Pancreatic | 850 | 850 | 750 | | 816.67 | 57.74 |
| | Adenocarcinoma | | | | | | |
| SK-OV3 | Ovarian | 550 | 535 | 520 | | 535.00 | 15.00 |
| | Adenocarcinoma | | | | | | |
| TOV-21G | Ovarian | 610 | 600 | 700 | | 636.67 | 55.08 |
| | Adenocarcinoma | | | | | | |
| OV-90 | Ovarian | 650 | 650 | 670 | | 656.67 | 11.55 |
| | Adenocarcinoma | | | | | | |
| OVCAR-3 | Ovarian | 650 | 650 | | | 650.00 | 0.00 |
| | Adenocarcinoma | | | | | | |
| PC3 | Prostate | 650 | 650 | 625 | | 641.67 | 14.43 |
| | Adenocarcinoma | | | | | | |
| CaCo-2 | Colon | 680 | 690 | 670 | | 680.00 | 10.00 |
| | Adenocarcinoma | | | | | | |
| MDA-MB-157 | Mammary medullary | 670 | 675 | 650 | | 665.00 | 13.23 |
| | tumour | | | | | | |
| HepG2 | Hepatoblastoma | 240 | 230 | 350 | | 273.33 | 66.58 |
| U2OS | Osteosarcoma | >1000* | | | | | |
| MES-SA | Uterine sarcoma | >1000* | | | | | |
| HT1080 | Fibrosarcoma | >1000* | | | | | |
| Canine | Hepatocyte | 240* | | | | | |
| Hepatocytes | | | | | | | |

*Study CXR0798;
**Study CXR0786;
All other data: Study CXR0859

The mechanism of cytotoxicity of APFO and CXR1002 was evaluated using bromodeoxyuridine (BrdU) incorporation to quantify cell proliferation and Hoechst 3342 staining to identify apoptotic cells. Significant suppression of BrdU incorporation was observed in all but one of the cell lines used in the SRB cytotoxicity assay following treatment with 300 μM APFO or CXR1002 for 48 hrs; in five cell lines, no proliferating cells were detectable at this concentration. No marked effects were observed at 10 μM, whereas the response to 30 μM was variable. The concentration dependence of induction of apoptosis was similar, with marked induction of apoptosis at 300 μM, little effect at 10 μM and variable responses at 30 μM.

### Example 4

### In Vivo Activity of CXR1002

CXR1002 has been examined in a small number of xenograft models, using both intra-peritoneal (i.p) and oral dosing (p.o). The effect of PFOA on HT-29 (colon adenocarcinoma) tumours was assessed in nude mouse xenografts, initially using APFO and subsequently using CXR1002. Animals were inoculated with a tumour cell suspension on each flank and the tumours were allowed to grow for 16 days. CXR1002 was administered intra-peritoneally three times per week for 28 days; results were graphed using a curve-fitting programme (FIG. 3). At 25 mg/kg, CXR1002 had an anti-tumour effect on HT-29 tumour volumes. No significant compound-dependent effects on body weight were detected (results not shown), but an increase in liver weight (up to 2.5 fold) was observed. The maximum plasma concentration of CXR1002 detected was 277 μM following this dosing regimen.

A parallel experiment was carried out using the prostate tumour cell line PC3. Xenograft tumours derived from PC3 cells grew much more slowly than HT-29 xenografts; nevertheless, CXR1002 had a marked anti-tumour effect in this model. The effects of different doses of CXR1002 (5, 15 and 25 mg/kg given by the i.p route) were very similar in this experiment, but for simplification, only data from the 25 mg/kg group is shown (FIG. 4). No marked effects on body weight were detected, but again an increase in liver weight was observed. The maximum plasma concentration of CXR1002 detected was 281 μM in mice treated with 25 mg/kg three times weekly.

In both the HT-29 and PC-3 xenograft experiments, slight reductions in plasma glucose and triglyceride levels were detected following CXR1002 treatment of tumour-bearing nude mice, consistent with activation of the PPARγ and PPARα receptors, respectively. Slight increases (up to 3.5 fold) in plasma AST occurred in response to CXR1002 in mice bearing either HT-29 or PC3 cell xenografts. Plasma ALT levels were only slightly increased in PC3-tumour bearing mice (up to 1.8 fold) and were actually decreased in mice bearing HT-29 xenografts. These effects are consistent with a transient effect on the liver associated with mild toxicity and reversible liver enlargement. In rodents, this type of effect is usually due to hepatic PPARα activation associated with peroxisome proliferation.

A further xenograft model was performed using the human pancreatic cell line PANC-1. This tumour is slow growing in vivo. Female nude mice were implanted with PANG-1 cells and once the tumours reached a pre-determined size the animals were dosed with CXR1002 at 25 mg/kg, 3 times per week. For various reasons, animals were lost during the study and the final group sizes were small. Nevertheless, the CXR1002 treated animals showed substantially delayed tumour growth and the weights and rigidity of the tumours were also different between the vehicle treated and untreated animals (FIG. 5, FIG. 6). This experiment is currently being repeated to try to obtain larger group sizes at experimental completion.

In-life and terminal blood samples taken from the mice were analysed for CXR1002 levels using a validated analytical method. In-life samples averaged 146 μM and terminal blood samples (24 hours post final dose) averaged 474 μM (FIG. 7). Plasma values were higher than the whole blood values. This may be attributed to the duration of dosing. In addition, CXR1002 is highly plasma protein bound. Furthermore, the erythrocyte/plasma partitioning coefficient (which measures the amount of drug bound to red cells compared to plasma binding) may contribute to the observed differences.

CXR1002 was also tested in a xenograft model of liver carcinoma using the cell line HepG2. In this experiment CXR1002 was dosed at 25 mg/kg in two different regimens: 2× per week and 3× per week. Although this tumour cell line is particularly sensitive to CXR1002 in vitro, the xenografted tumours showed a modest response in terms of growth inhibition. There was no obvious difference between the two different dosing regimens. The data in FIG. 8 and FIG. 9 shows the combined data from the 2 different treatment dosing regimens for tumour growth and tumour weight, respectively. The terminal plasma concentrations of CXR1002 were 437 μM for the 2× weekly regimen and 520 μM for the 3× weekly regimen.

To summarise, CXR1002 has been tested in four human tumour xenograft models, HT-29 (colon), PC3 (prostate), PANC-1 (pancreatic) and HepG2 (liver). Anti-tumour effects were detected in all models as shown in Table 6. No significant toxicity was observed, although there was evidence for minor changes in liver enzyme function, associated with a liver enlargement effect, which is probably rodent-specific. The exposure to CXR1002 in nude mice was lower than the blood levels achieved in patients at the higher doses in the CXR1002-001 phase I trial.

TABLE 6
Summary of best response in xenograft models

| | Absolute | | Terminal | |
|---|---|---|---|---|
| | Tumour Volume | | CXR1002 | |
| | as a Percentage | | plasma levels | In vitro 48 hr |
| Cell Line and | of Saline | Day of | (collected 24 hrs | IC₅₀ for |
| Tumour Model | Control* | evaluation | post last dose) | CXR1002 |
| HT-29 (Colon) | 49.22% | Day 29 | 277 μM | 341.7 ± 10.6** |
| | | | (Day 29) | |
| PC3 (Prostate) | 19.14% | Day 25 | 281 μM | 641.67 ± 14.43^{#} |
| | | | (Day 29) | |
| HepG2 (Liver) | 77.05% | Day 53 | 437 ± 127 μM (×2 | 273.33 ± 66.58^{#} |
| | | | weekly regimen) | |
| | | | 520 ± 46 μM | |
| | | | (×3 weekly | |
| | | | regimen) | |
| | | | (Days 60-64) | |
| PANC-1 | 50.13% | Day 82 | 474 ± 153 μM | >1000^{#} |
| (Pancreas) | | | (Day 88) | |

*Saline control = 100%
**Data from Table 4
^{#}Data from Table 5

### Other Relevant Pharmacology

PPARs play key roles in nutritional homeostasis, the primary effects of PPARα being in the regulation of fatty acid catabolism and those of PPARγ being in adipose differentiation and insulin-mediated regulation of glucose levels (2), (3). The hypolipidaemic effects of PPARα agonists are well characterised, while more recent studies have demonstrated the hypoglycaemic effects of PPARγ agonists (47), (48), (49), (50). While these effects may be peripheral to the anticancer effects of CXR1002, they are relevant as hypotriglyceridaemia and hypoglycaemia may be used as pharmacodynamic markers of PPAR α and γ agonism respectively.

### Example 5

### Human Clinical Data

CXR1002 monotherapy has been evaluated in a single Phase I trial in cancer patients with the primary objective of determining the maximum tolerated dose (MTD) of a weekly dosing schedule. A summary of this trial is provided in Table 7.

TABLE 7
Clinical Trial of CXR1002

| Protocol | Study | Dosing | | |
|---|---|---|---|---|
| Number | Design | Schedule | Dose Levels | Status |
| CXR1002-001 | Phase I | Weekly | single dose: 50 mg | Ongoing |
| | | dose | repeat dose: 50 mg, | (n = 43) |
| | | | 100 mg, 200 mg, | |
| | | | 300 mg, 450 mg, | |
| | | | 600 mg, 750 mg, | |
| | | | 950 mg, 1000 mg, | |
| | | | 1200 mg | |

CXR 1002 was administered in powder-filled hard gelatin capsules. One dose-strength oral capsules was used (50 mg).

The bulk active pharmaceutical ingredient will be manufactured under GMP conditions by Chimete Srl, Italy; and the capsules manufactured to cGMP by Penn Pharmaceutical Services LTD, UK.

Storage: All trial medication was held in a dry place at room temperature (15° C. to 25° C.) and protected from light.

The starting dose of CXR1002 was 50 mg administered orally as a single dose. This is approximately 0.24× the Lowest Observed Effect dose level in the monkey which is the most sensitive species that was tested.

CXR1002 was administered to patients, as a capsule by the oral route, orally as a single dose of 50 mg in the morning after an overnight fast in the first cohort of 3 patients. Prophylactic anti-emetics were not administered, and patients fasted for 1 hour after ingestion of CXR1002. PK samples, PD (fasting) samples, blood glucose, and blood triglyceride samples, were taken over a 6-week period.

These patients then underwent repeat dosing schedule with the same dose of CXR1002. The repeat dosing schedule was weekly administration of CXR1002 as a single oral dose in the morning and patients fasted for 1 hour before and after ingestion of CXR1002. Dose limiting toxicity (DLT) will be based on the toxicity assessments over the first 3-week period of the repeat dosing schedule. PK samples (single blood sample) were taken on the following basis:
Every 6 weeks during the repeat dosing phase
If dosing is interrupted or stopped, samples will be taken at intervals according to patient convenience
PK sampling for safety evaluation may take place at any time, as clinically indicated

In all dose cohorts subsequent to the initial dose cohort, all patients will be treated with weekly administration of study drug from the start of dosing. Dose escalation was performed after all patients at the preceding dose level had completed a 3-week repeat dosing period. The dose of CXR1002 was increased in successive dose cohorts until ≧Grade 2 drug-related toxicity was observed, after which dose escalation was in approximately 30% increments.

As of February, 2011, 43 patients with advanced cancers from one Phase I study have received CXR1002. The weekly dose administered ranges from 50 to 1200 mg.

The best response to CXR1002 treatment was stable disease by investigator assessment. One patient with pancreatic cancer had stable disease lasting 7 months.

Pharmacokinetic analysis of CXR1002 was carried out in the Phase I study using a validated assay. After oral administration of a single dose of CXR1002, the plasma concentration reached a Cmax at 1.5 hours in all 3 patients examined. After a single 50 mg dose the exposure in 3 patients varied between 8 and 16 μM and this was maintained at a constant level over the 6 week sampling period following the dose. The data indicates the half-life of elimination of CXR1002 could not be defined but is >6 weeks.

After weekly repeat doses of CXR1002 the plasma level increased in stepped increments. The maximal plasma level recorded to date was from a patient who had received a 1200 mg weekly dose over a 5 week period and had a plasma level of 1530 μM.

There appeared to be no gender difference in CXR1002 exposure following CXR1002 administration. The drug is eliminated extremely slowly and accumulates following a weekly dose.

Study CXR1002-001 is an open label, two centre, phase I study in patients with advanced cancer to assess the tolerability, safety and pharmacokinetics of CXR1002 administered weekly. The study synopsis is shown in Table 8.

TABLE 8
Study Synopsis for Study CXR1002-001 (n = 43)

| | Study Synopsis |
|---|---|
| Design | Open label |
| Study Period | First Patient In: 02Sep08 |
| | Last Patient Out: To be determined |
| | Study period: Approximately 3 years |
| Study Drug | CXR1002 50 mg capsule |
| Objectives | Primary: To determine the safety, toxicity and dose |
| | limiting toxicity (DLT) and maximum tolerated dose |
| | (MTD) of CXR1002 when given as single oral dose on a |
| | weekly schedule. |
| | Secondary: To describe the pharmacokinetics; to |
| | investigate the effect of CXR1002 on markers of PPAR |
| | agonist activity; to assess anti-tumour activity; to propose |
| | a safe dose for phase II. |
| Patient | Male and female patients with advanced solid tumours |
| Population | that are refractory to standard therapy or for which |
| | no standard therapy exists. |
| Number of | Planned: Up to 50 |
| Patients | Treated: 43 |
| (planned | |
| and treated) | |
| Drug | Oral |
| Administration | |
| Dosing | Single 50 mg dose |
| | Weekly dose; dose escalation starting at 50 mg |
| Dose | Up to 100% dose escalation permitted |
| Escalation | |
| Treatment | 6 weeks with pharmacokinetic sampling, thereafter as |
| Duration | long as patient receives a benefit |
| MTD | To be determined |

Forty three patients were enrolled in the study, as of February 2011. Thirty two patients were enrolled at the Beatson West of Scotland Cancer Centre, Glasgow, and eleven patients were enrolled at Aberdeen Royal Infirmary.

CXR1002 is being given orally as a weekly dose. The starting dose was a 50 mg single dose. The starting weekly repeat dose was 50 mg, with 2 patients continuing to the repeat dose schedule after receiving a single dose. Doses were escalated in groups of three patients. The dose escalation is continuing. A summary of the dose escalation is provided in Table 9.

TABLE 9
Dose Escalation Summary (n = 43)

| Dose (mg/week) | Number of Patients |
|---|---|
| 50 | 4 (1 patient received only single dose) |
| 100 | 3 |
| 200 | 3 |
| 300 | 4 |
| 450 | 3 |
| 600 | 7 |
| 750 | 3 |
| 950 | 4 |
| 1000 | 5 |
| 1200 | 6 |

A validated analytical assay consisting of non-GLP LC-MS/MS was used to quantitate CXR1002 in human plasma. Plasma samples were collected after the single 50 mg dose at the following timepoints: Pre-dose, and then 0.25, 0.5, 0.75, 1, 1.5, 2, 3, 4, 6, 24, 48, and 72 hours after administration and then once weekly at weeks 2, 3, 4, 5, and 6 (days 8, 15, 22, 29, and 36). For patients treated with the weekly repeat dose, plasma samples were collected at the following timepoints: Pre-dose and then 2, 3, 4 and 24 hours after administration for a total of 6 weeks. Thereafter a single sample was collected every 6 weeks for monitoring of exposure during long term treatment. Plasma samples were processed at site and stored at −80° C. prior to batch shipment to the analytical laboratory.

Of the 43 patients enrolled in study CXR1002-001, 24 were males and 19 were females. The majority of patients had received 2 prior therapies. Two patients had received 5 prior therapies. The tumour types of the patients are shown in Table 10.

TABLE 10
Patient demographics: Tumour type on Study CXR1002-001 (n = 43)

| Cancer Type | Number of Patients |
|---|---|
| pancreatic | 6 |
| parathyroid | 1 |
| gastric | 1 |
| renal | 2 |
| colorectal | 17 |
| breast | 1 |
| cervix | 1 |
| anaplastic thyroid | 1 |
| vulval | 1 |
| lung | 1 |
| oesophageal | 3 |
| unknown primary | 3 |
| carcinoid | 1 |
| melanoma | 2 |
| sarcoma | 1 |
| nasopharyngeal | 1 |
| carcinoma | |

Pharmacodynamic samples were also collected from patients for the measurement of pharmacodynamic markers. Samples were collected using the same time schedule as that used for the pharmacokinetic samples.

### Pharmacokinetic Sample Analysis

The following data shows for each patient the plasma levels over time. The particular weekly dose is shown, as is the gender and age of each patient. Graphical plots of the data for each patient are shown in FIGS. 10 to 27, 67 to 70 and 83 to 101.

TABLE 11
(a-an)
(a) Patient 001
Date of Birth: 07.07.1944
Dose: 50 mg
Sex: Male

| | | | Con- | | Con- |
|---|---|---|---|---|---|
| Time | Concentration | Time | centration | Time | centration |
| (hr) | (μM) | (hr) | (μM) | (hr) | (μM) |
| 0 | 0.00 | 24 | 13.81 | Day 227 | 307.9 |
| 0.25 | 0.35 | 48 | 12.76 | Day 268 | 268.0 |
| 0.5 | 1.11 | 72 | 9.70 | | |
| 0.75 | 9.17 | 192 | 8.54 | | |
| | | (Day 8) | | | |
| 1 | 14.41 | 360 | 8.63 | | |
| | | (Day 15) | | | |
| 1.5 | 25.72 | 528 | 11.58 | | |
| | | (Day 22) | | | |
| 2 | 22.48 | 696 | 10.23 | | |
| | | (Day 29) | | | |
| 3 | 20.82 | 864 | 8.89 | | |
| | | (Day 36) | | | |
| 4 | 18.19 | Day 144 | 226.2 | | |
| 6 | 14.67 | Day 179 | 250.5 | | |

(b) Patient 002
Date of Birth: 21.05.1950
Dose: 50 mg
Sex: Female

| Time | Concentration | Time | Concentration |
|---|---|---|---|
| (hr) | (μM) | (hr) | (μM) |
| 0 | 0.00 | 6 | 17.52 |
| 0.25 | 3.06 | 24 | 14.27 |
| 0.5 | 7.62 | 48 | 13.28 |
| 0.75 | 8.39 | 72 | 15.60 |
| 1 | 8.55 | 192 | 17.15 |
| | | (Day 8) | |
| 1.5 | 29.79 | 360 | 18.61 |
| | | (Day 15) | |
| 2 | 24.07 | 528 | 21.47 |
| | | (Day 22) | |
| 3 | 22.76 | 696 | 20.96 |
| | | (Day 29) | |
| 4 | 14.45 | 864 | 20.08 |
| | | (Day 36) | |

(c) Patient 003
Date of Birth: 29.12.1933
Dose: 50 mg
Sex: Male

| Time | Concentration | Time | Concentration |
|---|---|---|---|
| (hr) | (μM) | (hr) | (μM) |
| 0 | 0.14 | 6 | 16.90 |
| 0.25 | 1.08 | 24 | 19.53 |
| 0.5 | 11.30 | 48 | 18.07 |
| 0.75 | 17.05 | 72 | 18.43 |
| 1 | 20.69 | 192 | 8.60 |
| | | (Day 8) | |
| 1.5 | 24.64 | 360 | 7.20 |
| | | (Day 15) | |
| 2 | 24.49 | 528 | 6.50 |
| | | (Day 22) | |
| 3 | 21.15 | 696 | 5.00 |
| | | (Day 29) | |
| 4 | 17.98 | 864 | 6.50 |
| | | (Day 36) | |

(d) Patient 004
Date of Birth: 15.09.1954
Dose: 50 mg
Sex: Female

| | | | Con- | | Con- |
|---|---|---|---|---|---|
| Time | Concentration | Time | centration | Time | centration |
| (hr) | (μM) | (hr) | (μM) | (hr) | (μM) |
| 0 | 0.10 | 336 | 21.81 | 672 | 52.28 |
| | | (Day 15) | | (Day 29) | |
| 2 | 19.95 | 338 | 31.09 | 674 | 65.78 |
| 3 | 16.94 | 339 | 38.75 | 675 | 61.74 |
| 4 | 19.26 | 340 | 40.60 | 676 | 56.69 |
| 24 | 14.39 | 360 | 38.75 | 696 | 77.49 |
| (Day 2) | | (Day 16) | | (Day 30) | |
| 168 | 12.76 | 504 | 33.37 | 840 | 63.04 |
| (Day 8) | | (Day 22) | | (Day 36) | |
| 170 | 35.73 | 506 | 46.29 | 842 | 78.63 |
| 171 | 40.37 | 507 | 50.41 | 843 | 80.55 |
| 172 | 35.96 | 508 | 47.88 | 844 | No sample |
| 192 | 22.51 | 528 | 49.96 | 864 | 81.07 |
| (Day 9) | | (Day 23) | | (Day 37) | |

(e) Patient 005
Date of Birth: 27.02.1941
Dose: 100 mg
Sex: Male

| | Concen- | | | | Con- |
|---|---|---|---|---|---|
| Time | tration | Time | Concentration | Time | centration |
| (hr) | (μM) | (hr) | (μM) | (hr) | (μM) |
| 0 | 0.23 | 338 | 80.20 | 675 | 100.84 |
| 2 | 19.83 | 339 | 70.09 | 676 | 82.74 |
| 3 | 23.01 | 340 | 53.92 | 696 | 78.80 |
| | | | | (Day 30) | |
| 4 | 23.66 | 360 | 47.40 | 840 | 99.43 |
| | | (Day 16) | | (Day 36) | |
| 24 | 19.94 | 504 | 48.39 | 842 | 79.99 |
| (Day 2) | | (Day 22) | | | |
| 168 | 18.96 | 506 | 84.00 | 843 | 98.91 |
| (Day 8) | | | | | |
| 170 | 42.88 | 507 | 74.40 | 844 | 91.44 |
| 171 | 50.82 | 508 | 87.35 | 864 | 109.10 |
| | | | | (Day 37) | |
| 172 | 45.71 | 528 | 57.87 | Day 92 | 276.33 |
| | | (Day 23) | | | |
| 192 | 34.37 | 672 | 80.77 | | |
| (Day 9) | | (Day 29) | | | |
| 336 | 47.82 | 674 | 99.65 | | |
| (Day 15) | | | | | |

(f) Patient 006
Date of Birth: 12.04.1943
Dose: 100 mg
Sex: Male

| | Concen- | | | | Con- |
|---|---|---|---|---|---|
| Time | tration | Time | Concentration | Time | centration |
| (hr) | (μM) | (hr) | (μM) | (hr) | (μM) |
| 0 | 0.21 | 336 | 33.10 | 672 | 86.94 |
| | | (Day 15) | | (Day 29) | |
| 2 | 32.32 | 338 | 49.50 | 674 | 85.73 |
| 3 | 27.07 | 339 | 55.91 | 675 | n.s |
| 4 | 24.62 | 340 | n.s | 676 | n.s |
| 24 | 28.18 | 360 | 65.25 | 696 | 89.54 |
| (Day 2) | | (Day 16) | | (Day 30) | |
| 168 | 26.16 | 504 | 70.55 | 840 | 85.61 |
| (Day 8) | | (Day 22) | | (Day 36) | |
| 170 | 36.65 | 506 | 76.44 | 842 | 179.07 |
| 171 | 41.15 | 507 | 83.83 | 843 | 137.87 |
| 172 | 47.47 | 508 | n.s | 844 | 138.38 |
| 192 | 44.66 | 528 | 97.00 | 864 | 122.64 |
| (Day 9) | | (Day 23) | | (Day 37) | |

(g) Patient 007
Date of Birth: 06.01.1963
Dose: 100 mg
Sex: Female

| | Concen- | | | | Con- |
|---|---|---|---|---|---|
| Time | tration | Time | Concentration | Time | centration |
| (hr) | (μM) | (hr) | (μM) | (hr) | (μM) |
| 0 | 0.00 | 336 | 19.23 | 672 | |
| | | (Day 15) | | (Day 29) | |
| 2 | 30.91 | 338 | 52.84 | 674 | |
| 3 | 28.55 | 339 | 53.14 | 675 | |
| 4 | 19.26 | 340 | 55.78 | 676 | |
| 24 | 17.29 | 360 | 32.01 | 696 | |
| (Day 2) | | (Day 16) | | (Day 30) | |
| 168 | 12.23 | 504 | 39.69 | 840 | |
| (Day 8) | | (Day 22) | | (Day 36) | |
| 170 | n.s | 506 | 53.12 | 842 | |
| 171 | n.s | 507 | 65.79 | 843 | |
| 172 | n.s | 508 | 73.03 | 844 | |
| 192 | n.s | 528 | 63.42 | 864 | |
| (Day 9) | | (Day 23) | | (Day 37) | |

(h) Patient 008
Date of Birth: 21.01.1940
Dose: 200 mg
Sex: Male

| | Concen- | | | | Con- |
|---|---|---|---|---|---|
| Time | tration | Time | Concentration | Time | centration |
| (hr) | (μM) | (hr) | (μM) | (hr) | (μM) |
| 0 | 0.00 | 336 | 124.19 | 672 | 337.46 |
| | | (Day 15) | | (Day 29) | |
| 2 | 114.25 | 338 | 147.04 | 674 | 355.90 |
| 3 | 102.46 | 339 | 172.18 | 675 | 321.30 |
| 4 | 81.02 | 340 | 191.50 | 676 | 364.45 |
| 24 | 70.37 | 360 | 210.97 | 696 | 426.16 |
| (Day 2) | | (Day 16) | | (Day 30) | |
| 168 | 64.56 | 504 | 276.84 | 840 | 424.66 |
| (Day 8) | | (Day 22) | | (Day 36) | |
| 170 | 171.02 | 506 | 282.15 | 842 | 360.53 |
| 171 | 142.88 | 507 | 368.27 | 843 | 396.83 |
| 172 | 129.91 | 508 | 280.95 | 844 | 414.33 |
| 192 | 110.01 | 528 | 284.86 | 864 | 354.39 |
| (Day 9) | | (Day 23) | | (Day 37) | |

(i) Patient 009
Date of Birth: 11.03.1973
Dose: 200 mg
Sex: Female

| | Concen- | | Concen- | | Concen- |
|---|---|---|---|---|---|
| Time | tration | Time | tration | Time | tration |
| (hr) | (μM) | (hr) | (μM) | (hr) | (μM) |
| 0 | 0.00 | 338 | 161.42 | 675 | 261.46 |
| 2 | 93.43 | 339 | 167.24 | 676 | 358.20 |
| 3 | 61.21 | 340 | 152.60 | 696 | 338.09 |
| | | | | (Day 30) | |
| 4 | 71.13 | 360 | 218.26 | 840 | 399.10 |
| | | (Day 16) | | (Day 36) | |
| 24 | 64.58 | 504 | 253.19 | 842 | 345.61 |
| (Day 2) | | (Day 22) | | | |
| 168 | 60.06 | 506 | 285.24 | 843 | 373.31 |
| (Day 8) | | | | | |
| 170 | 148.65 | 507 | 295.84 | 844 | 329.34 |
| 171 | 170.29 | 508 | 362.32 | 864 | 346.17 |
| | | | | (Day 37) | |
| 172 | 138.60 | 528 | 241.69 | Day 92 | 456.38 |
| | | (Day 23) | | | |
| 192 | 136.45 | 672 | 251.16 | Week 19 | 617.56 |
| (Day 9) | | (Day 29) | | | |
| 336 | 127.41 | 674 | 471.59 | Week 25 | 500.93 |
| (Day 15) | | | | | |
| | | | | Week 32 | 540.20 |

(j) Patient 010
Date of Birth: 29.01.1959
Dose: 200 mg
Sex: Male

| | Concen- | | Concen- | | Concen- |
|---|---|---|---|---|---|
| Time | tration | Time | tration | Time | tration |
| (hr) | (μM) | (hr) | (μM) | (hr) | (μM) |
| 0 | 0.00 | 336 | 98.79 | 672 | 192.34 |
| | | (Day 15) | | (Day 29) | |
| 2 | 21.07 | 338 | 118.05 | 674 | 192.67 |
| 3 | 30.35 | 339 | 103.02 | 675 | 236.98 |
| 4 | 33.61 | 340 | 66.68 | 676 | 230.90 |
| 24 | 58.60 | 360 | 174.52 | 696 | 256.06 |
| (Day 2) | | (Day 16) | | (Day 30) | |
| 168 | 51.00 | 504 | 181.86 | 840 | 242.97 |
| (Day 8) | | (Day 22) | | (Day 36) | |
| 170 | 81.72 | 506 | 207.19 | 842 | 188.17 |
| 171 | 81.39 | 507 | 276.15 | 843 | 232.44 |
| 172 | 119.44 | 508 | 186.77 | 844 | 222.78 |
| 192 | 101.39 | 528 | 190.86 | 864 | 220.65 |
| (Day 9) | | (Day 23) | | (Day 37) | |

(k) Patient 011
Date of Birth: 15.04.1961
Dose: 300 mg
Sex: Male

| | Concen- | | Concen- | | Concen- |
|---|---|---|---|---|---|
| Time | tration | Time | tration | Time | tration |
| (hr) | (μM) | (hr) | (μM) | (hr) | (μM) |
| 0 | n.d | 336 | 131.32 | 672 | 254.90 |
| | | (Day 15) | | (Day 29) | |
| 2 | 100.88 | 338 | 159.58 | 674 | 291.09 |
| 3 | 107.29 | 339 | 160.00 | 675 | 304.27 |
| 4 | 111.65 | 340 | 146.91 | 676 | 242.69 |
| 24 | 95.87 | 360 | 237.26 | 696 | 326.13 |
| (Day 2) | | (Day 16) | | (Day 30) | |
| 168 | 57.87 | 504 | 145.38 | 840 | 269.65 |
| (Day 8) | | (Day 22) | | (Day 36) | |
| 170 | 112.25 | 506 | 203.57 | 842 | 384.08 |
| 171 | 122.41 | 507 | 288.21 | 843 | 350.35 |
| 172 | 138.33 | 508 | 243.62 | 844 | 282.27 |
| 192 | 178.42 | 528 | 241.39 | 864 | 386.77 |
| (Day 9) | | (Day 23) | | (Day 37) | |
| | | | | Week 12 | 573.70 |

(l) Patient 012
Date of Birth: 19.06.1945
Dose: 300 mg
Sex: Male

| | Concen- | | Concen- | | Concen- |
|---|---|---|---|---|---|
| Time | tration | Time | tration | Time | tration |
| (hr) | (μM) | (hr) | (μM) | (hr) | (μM) |
| 0 | n.d | 336 | 141.25 | 672 | 303.06 |
| | | (Day 15) | | (Day 29) | |
| 2 | 91.38 | 338 | 240.93 | 674 | 340.42 |
| 3 | 93.53 | 339 | 196.29 | 675 | 372.99 |
| 4 | 122.90 | 340 | 213.09 | 676 | 291.97 |
| 24 | 74.05 | 360 | 218.10 | 696 | 334.20 |
| (Day 2) | | (Day 16) | | (Day 30) | |
| 168 | 64.83 | 504 | 180.56 | 840 | |
| (Day 8) | | (Day 22) | | (Day 36) | |
| 170 | 182.32 | 506 | 277.77 | 842 | |
| 171 | 147.52 | 507 | 260.84 | 843 | |
| 172 | 126.45 | 508 | 221.25 | 844 | |
| 192 | 151.74 | 528 | 263.16 | 864 | |
| (Day 9) | | (Day 23) | | (Day 37) | |

(m) Patient 013
Date of Birth: 04.09.1957
Dose: 300 mg
Sex: Female

| | Concen- | | Concen- | | |
|---|---|---|---|---|---|
| Time | tration | Time | tration | Time | Concentration |
| (hr) | (μM) | (hr) | (μM) | (hr) | (μM) |
| 0 | n.d | 336 | | 672 | |
| | | (Day 15) | | (Day 29) | |
| 2 | 82.72 | 338 | | 674 | |
| 3 | 75.97 | 339 | | 675 | |
| 4 | 85.32 | 340 | | 676 | |
| 24 | 79.61 | 360 | | 696 | |
| (Day 2) | | (Day 16) | | (Day 30) | |
| 168 | | 504 | | 840 | |
| (Day 8) | | (Day 22) | | (Day 36) | |
| 170 | | 506 | | 842 | |
| 171 | | 507 | | 843 | |
| 172 | | 508 | | 844 | |
| 192 | | 528 | | 864 | |
| (Day 9) | | (Day 23) | | (Day 37) | |

(n) Patient 014
Date of Birth: 01.09.1937
Dose: 300 mg
Sex: Male

| | Concen- | | Concen- | | |
|---|---|---|---|---|---|
| Time | tration | Time | tration | Time | Concentration |
| (hr) | (μM) | (hr) | (μM) | (hr) | (μM) |
| 0 | n.d. | 336 | 173.32 | 672 | 354.70 |
| | | (Day 15) | | (Day 29) | |
| 2 | 131.24 | 338 | 297.35 | 674 | 389.10 |
| 3 | 120.77 | 339 | 270.41 | 675 | 399.93 |
| 4 | 100.33 | 340 | 261.42 | 676 | 411.42 |
| 24 | 101.72 | 360 | n.s | 696 | 478.38 |
| (Day 2) | | (Day 16) | | (Day 30) | |
| 168 | 79.64 | 504 | 287.79 | 840 | 425.93 |
| (Day 8) | | (Day 22) | | (Day 36) | |
| 170 | 154.12 | 506 | 335.79 | 842 | 506.31 |
| 171 | 178.41 | 507 | 373.83 | 843 | 520.08 |
| 172 | 179.97 | 508 | n.s | 844 | 562.63 |
| 192 | 178.39 | 528 | 420.49 | 864 | 487.60 |
| (Day 9) | | (Day 23) | | (Day 37) | |
| | | | | Week 12 | 889.60 |
| | | | | Week 18 | 979.62 |

(o) Patient 015
Date of Birth: 26.06.1939
Dose: 450 mg
Sex: Female

| | Concen- | | Concen- | | |
|---|---|---|---|---|---|
| Time | tration | Time | tration | Time | Concentration |
| (hr) | (μM) | (hr) | (μM) | (hr) | (μM) |
| 0 | n.d. | 336 | 286.27 | 672 | 543.57 |
| | | (Day 15) | | (Day 29) | |
| 2 | 176.20 | 338 | 463.43 | 674 | 622.36 |
| 3 | 231.36 | 339 | 393.32 | 675 | 632.84 |
| 4 | 216.70 | 340 | 424.29 | 676 | 675.19 |
| 24 | 126.15 | 360 | 411.72 | 696 | 707.80 |
| (Day 2) | | (Day 16) | | (Day 30) | |
| 168 | 137.59 | 504 | 386.72 | 840 | 575.19 |
| (Day 8) | | (Day 22) | | (Day 36) | |
| 170 | 324.96 | 506 | 578.86 | 842 | 702.79 |
| 171 | 314.27 | 507 | 548.01 | 843 | 655.16 |
| 172 | n.s. | 508 | 513.31 | 844 | 800.55 |
| 192 | 294.21 | 528 | 550.55 | 864 | 592.16 |
| (Day 9) | | (Day 23) | | (Day 37) | |
| | | | | Week 13 | 1174.82 |

(p) Patient 016
Date of Birth: 11.11.1957
Dose: 450 mg
Sex: Female

| | Concen- | | Concen- | | |
|---|---|---|---|---|---|
| Time | tration | Time | tration | Time | Concentration |
| (hr) | (μM) | (hr) | (μM) | (hr) | (μM) |
| 0 | n.d. | 336 | 286.80 | 672 | 605.12 |
| | | (Day 15) | | (Day 29) | |
| 2 | 107.59 | 338 | 417.75 | 674 | 906.59 |
| 3 | 125.90 | 339 | 454.11 | 675 | 745.49 |
| 4 | 88.14 | 340 | 442.28 | 676 | 732.34 |
| 24 | 164.05 | 360 | 470.53 | 696 | 871.19 |
| (Day 2) | | (Day 16) | | (Day 30) | |
| 168 | 181.57 | 504 | 545.74 | 840 | |
| (Day 8) | | (Day 22) | | (Day 36) | |
| 170 | 327.77 | 506 | 693.48 | 842 | |
| 171 | 255.06 | 507 | 721.48 | 843 | |
| 172 | 243.61 | 508 | 697.87 | 844 | |
| 192 | 348.41 | 528 | 692.36 | 864 | |
| (Day 9) | | (Day 23) | | (Day 37) | |

(q) Patient 017
Date of Birth: 09.12.1933
Dose: 450 mg
Sex: Female

| | Con- | | | | Con- |
|---|---|---|---|---|---|
| Time | centration | Time | Concentration | Time | centration |
| (hr) | (μM) | (hr) | (μM) | (hr) | (μM) |
| 0 | n.d. | 336 | 216.41 | 672 | 403.72 |
| | | (Day 15) | | (Day 29) | |
| 2 | 99.68 | 338 | 325.48 | 674 | 451.12 |
| 3 | 136.14 | 339 | 341.96 | 675 | 497.22 |
| 4 | 163.18 | 340 | No sample | 676 | 484.61 |
| 24 | 127.31 | 360 | 258.49 | 696 | 483.20 |
| (Day 2) | | (Day 16) | | (Day 30) | |
| 168 | 146.02 | 504 | 332.56 | 840 | 434.00 |
| (Day 8) | | (Day 22) | | (Day 36) | |
| 170 | 276.16 | 506 | 427.08 | 842 | 460.92 |
| 171 | 252.67 | 507 | 411.89 | 843 | 456.72 |
| 172 | 248.08 | 508 | No sample | 844 | 482.23 |
| 192 | 245.77 | 528 | 405.28 | 864 | 525.98 |
| (Day 9) | | (Day 23) | | (Day 37) | |

(r) Patient 018
Date of Birth: 23.05.1940
Dose: 600 mg
Sex: Female

| | Con- | | | | Con- |
|---|---|---|---|---|---|
| Time | centration | Time | Concentration | Time | centration |
| (hr) | (μM) | (hr) | (μM) | (hr) | (μM) |
| 0 | n.d. | 336 | 356.38 | 672 | |
| | | (Day 15) | | (Day 29) | |
| 2 | 338.52 | 338 | 551.28 | 674 | |
| 3 | 280.28 | 339 | 563.34 | 675 | |
| 4 | 248.26 | 340 | 590.95 | 676 | |
| 24 | 213.23 | 360 | 553.60 | 696 | |
| (Day 2) | | (Day 16) | | (Day 30) | |
| 168 | 180.97 | 504 | | 840 | |
| (Day 8) | | (Day 22) | | (Day 36) | |
| 170 | 397.91 | 506 | | 842 | |
| 171 | 406.73 | 507 | | 843 | |
| 172 | n.s. | 508 | | 844 | |
| 192 | 385.85 | 528 | | 864 | |
| (day 9) | | (Day 23) | | (Day 37) | |

(s) Patient 020
Date of Birth: 20.04.1966
Dose: 600 mg
Sex: Male

| | Con- | | | | Con- |
|---|---|---|---|---|---|
| Time | centration | Time | Concentration | Time | centration |
| (hr) | (μM) | (hr) | (μM) | (hr) | (μM) |
| 0 | n.d. | 336 | 271.93 | 672 | 540.35 |
| | | (Day 15) | | (Day 29) | |
| 2 | 207.66 | 338 | 474.01 | 674 | 547.27 |
| 3 | 182.13 | 339 | 433.87 | 675 | 603.48 |
| 4 | 179.81 | 340 | 437.8 | 676 | 651.85 |
| 24 | 413.39 | 360 | 410.21 | 696 | 615.23 |
| (Day 2) | | (Day 16) | | (Day 30) | |
| 168 | 125.29 | 504 | 440.64 | 840 | 582.17 |
| (Day 8) | | (Day 22) | | (Day 36) | |
| 170 | 327.38 | 506 | 477.40 | 842 | 700.19 |
| 171 | 309.51 | 507 | 526.45 | 843 | 764.41 |
| 172 | 278.89 | 508 | 536.35 | 844 | 770.32 |
| 192 | 268.45 | 528 | 562.88 | 864 | 702.75 |
| (day 9) | | (Day 23) | | (Day 37) | |

(t) Patient 021
Date of Birth: 28.08.1958
Dose: 600 mg
Sex: Female

| | Con- | | | | Con- |
|---|---|---|---|---|---|
| Time | centration | Time | Concentration | Time | centration |
| (hr) | (μM) | (hr) | (μM) | (hr) | (μM) |
| 0 | n.d. | 336 | 412.22 | 672 | 613.22 |
| | | (Day 15) | | (Day 29) | |
| 2 | 77.23 | 338 | 649.84 | 674 | 681.46 |
| 3 | 120.90 | 339 | 652.79 | 675 | 847.13 |
| 4 | 117.32 | 340 | 633.0 | 676 | 845.20 |
| 24 | 203.29 | 360 | 604.18 | 696 | 799.22 |
| (Day 2) | | (Day 16) | | (Day 30) | |
| 168 | 217.79 | 504 | 604.54 | 840 | 778.84 |
| (Day 8) | | (Day 22) | | (Day 36) | |
| 170 | 504.50 | 506 | 734.36 | 842 | 968.41 |
| 171 | 416.60 | 507 | 654.13 | 843 | 927.77 |
| 172 | 450.70 | 508 | 721.72 | 844 | 980.72 |
| 192 | 478.75 | 528 | 677.90 | 864 | 995.39 |
| (Day 9) | | (Day 23) | | (Day 37) | |

(u) Patient 022
Date of Birth: 11.05.1959
Dose: 600 mg
Sex: Male

| | Con- | | Con- | | |
|---|---|---|---|---|---|
| Time | centration | Time | centration | Time | Concentration |
| (hr) | (μM) | (hr) | (μM) | (hr) | (μM) |
| 0 | n.d. | 336 | 289.10 | 672 | |
| | | (Day 15) | | (Day 29) | |
| 2 | 192.81 | 338 | 433.41 | 674 | |
| 3 | 196.01 | 339 | 433.35 | 675 | |
| 4 | 198.74 | 340 | 405.3 | 676 | |
| 24 | 156.54 | 360 | 427.22 | 696 | |
| (Day 2) | | (Day 16) | | (Day 30) | |
| 168 | 143.49 | 504 | 426.00 | 840 | |
| (Day 8) | | (Day 22) | | (Day 36) | |
| 170 | 309.80 | 506 | 441.08 | 842 | |
| 171 | 269.82 | 507 | 561.63 | 843 | |
| 172 | 275.79 | 508 | 518.62 | 844 | |
| 192 | 298.41 | 528 | 595.95 | 864 | |
| (day 9) | | (Day 23) | | (Day 37) | |

(v) Patient 023
Date of Birth: 10.06.1940
Dose: 600 mg
Sex: Male

| | Con- | | Con- | | |
|---|---|---|---|---|---|
| Time | centration | Time | centration | Time | Concentration |
| (hr) | (μM) | (hr) | (μM) | (hr) | (μM) |
| 0 | n.d. | 336 | 431.55 | 672 | |
| | | (Day 15) | | (Day 29) | |
| 2 | 208.10 | 338 | 494.51 | 674 | |
| 3 | 233.12 | 339 | 613.01 | 675 | |
| 4 | 236.13 | 340 | 635.73 | 676 | |
| 24 | 221.19 | 360 | 616.06 | 696 | |
| (Day 2) | | (Day 16) | | (Day 30) | |
| 168 | 204.11 | 504 | | 840 | |
| (Day 8) | | (Day 22) | | (Day 36) | |
| 170 | 335.70 | 506 | | 842 | |
| 171 | 336.75 | 507 | | 843 | |
| 172 | 390.84 | 508 | | 844 | |
| 192 | 400.07 | 528 | | 864 | |
| (day 9) | | (Day 23) | | (Day 37) | |

(w) Patient 024
Date of Birth: 14.09.1939
Dose: 600 mg
Sex: Female

| | Con- | | Con- | | |
|---|---|---|---|---|---|
| Time | centration | Time | centration | Time | Concentration |
| (hr) | (μM) | (hr) | (μM) | (hr) | (μM) |
| 0 | n.d. | 336 | 488.31 | 672 | 788.40 |
| | | (Day 15) | | (Day 29) | |
| 2 | 91.59 | 338 | 577.60 | 674 | 693.74 |
| 3 | 187.52 | 339 | 532.72 | 675 | 779.35 |
| 4 | 282.55 | 340 | 628.20 | 676 | 780.28 |
| 24 | 257.81 | 360 | 647.35 | 696 | 810.90 |
| (Day 2) | | (Day 16) | | (Day 30) | |
| 168 | 211.59 | 504 | 691.46 | 840 | 813.92 |
| (Day 8) | | (Day 22) | | (Day 36) | |
| 170 | 435.55 | 506 | 642.15 | 842 | 897.68 |
| 171 | 440.70 | 507 | 705.87 | 843 | 903.71 |
| 172 | 439.24 | 508 | 630.45 | 844 | 908.35 |
| 192 | 418.38 | 528 | 858.92 | 864 | 966.13 |
| (Day 9) | | (Day 23) | | (Day 37) | |

(y) Patient 026
Date of Birth: 23.07.1951
Dose: 750 mg
Sex: Male

| | Con- | | Con- | | |
|---|---|---|---|---|---|
| Time | centration | Time | centration | Time | Concentration |
| (hr) | (μM) | (hr) | (μM) | (hr) | (μM) |
| 0 | n.d. | 336 | 352.35 | 672 | 593.90 |
| | | (Day 15) | | (Day 29) | |
| 2 | 95.82 | 338 | 399.42 | 674 | 605.61 |
| 3 | 124.60 | 339 | 505.65 | 675 | 655.20 |
| 4 | 149.03 | 340 | 492.30 | 676 | 697.97 |
| 24 | 200.07 | 360 | 624.63 | 696 | 732.46 |
| (Day 2) | | (Day 16) | | (Day 30) | |
| 168 | 185.64 | 504 | 478.99 | 840 | 728.08 |
| (Day 8) | | (Day 22) | | (Day 36) | |
| 170 | 270.45 | 506 | 625.39 | 842 | 823.68 |
| 171 | 301.45 | 507 | 546.33 | 843 | 775.85 |
| 172 | 339.13 | 508 | 581.68 | 844 | 821.51 |
| 192 | 397.76 | 528 | 607.97 | 864 | 811.72 |
| (Day 9) | | (Day 23) | | (Day 37) | |

(z) Patient 027
Date of Birth: 17.10.1943
Dose: 750 mg
Sex: Female

| | Con- | | Con- | | |
|---|---|---|---|---|---|
| Time | centration | Time | centration | Time | Concentration |
| (hr) | (μM) | (hr) | (μM) | (hr) | (μM) |
| 0 | n.d. | 336 | 365.66 | 672 | 605.65 |
| | | (Day 15) | | (Day 29) | |
| 2 | 109.52 | 338 | 384.07 | 674 | 768.97 |
| 3 | 240.51 | 339 | 411.73 | 675 | 811.16 |
| 4 | 224.48 | 340 | 451.09 | 676 | 756.91 |
| 24 | 199.39 | 360 | 569.22 | 696 | 803.44 |
| (Day 2) | | (Day 16) | | (Day 30) | |
| 168 | 167.86 | 504 | 476.33 | 840 | 672.92 |
| (Day 8) | | (Day 22) | | (Day 36) | |
| 170 | 235.24 | 506 | 658.10 | 842 | |
| 171 | 317.57 | 507 | 668.76 | 843 | |
| 172 | 344.46 | 508 | 671.26 | 844 | |
| 192 | 410.69 | 528 | 719.70 | 864 | |
| (Day 9) | | (Day 23) | | (Day 37) | |

(aa) Patient 028
Date of Birth: 29.07.1944
Dose: 750 mg
Sex: Male

| | Con- | | Con- | | |
|---|---|---|---|---|---|
| Time | centration | Time | centration | Time | Concentration |
| (hr) | (μM) | (hr) | (μM) | (hr) | (μM) |
| 0 | n.d. | 336 | 288.21 | 672 | 521.82 |
| | | (Day 15) | | (Day 29) | |
| 2 | 206.86 | 338 | 472.99 | 674 | 709.85 |
| 3 | 173.85 | 339 | 395.89 | 675 | 713.40 |
| 4 | 160.39 | 340 | 405.8 | 676 | 700.01 |
| 24 | 159.66 | 360 | 450.13 | 696 | 757.67 |
| (Day 2) | | (Day 16) | | (Day 30) | |
| 168 | 160.74 | 504 | 467.30 | 840 | 682.49 |
| (Day 8) | | (Day 22) | | (Day 36) | |
| 170 | 321.26 | 506 | 581.50 | 842 | 824.29 |
| 171 | 301.29 | 507 | 489.55 | 843 | 791.81 |
| 172 | 272.96 | 508 | 581.26 | 844 | 853.05 |
| 192 | 295.12 | 528 | 654.60 | 864 | 774.65 |
| (Day 9) | | (Day 23) | | (Day 37) | |

(ab) Patient 029
Date of Birth: 19.05.1946
Dose: 950 mg
Sex: Female

| | Con- | | Con- | | |
|---|---|---|---|---|---|
| Time | centration | Time | centration | Time | Concentration |
| (hr) | (μM) | (hr) | (μM) | (hr) | (μM) |
| 0 | n.d. | 336 | 438.90 | 672 | 492.32 |
| | | (Day 15) | | (Day 29) | |
| 2 | 218.65 | 338 | 703.63 | 674 | 841.18 |
| 3 | 250.85 | 339 | 777.16 | 675 | 971.71 |
| 4 | 307.26 | 340 | 673.88 | 676 | 812.35 |
| 24 | 352.58 | 360 | 896.30 | 696 | 949.34 |
| (Day 2) | | (Day 16) | | (Day 30) | |
| 168 | 288.92 | 504 | 400.09 | 840 | 777.26 |
| (Day 8) | | (Day 22) | | (Day 36) | |
| 170 | 603.87 | 506 | 896.90 | 842 | 792.81 |
| 171 | 601.54 | 507 | 741.47 | 843 | n.s. |
| 172 | 556.45 | 508 | 700.77 | 844 | n.s. |
| 192 | 606.03 | 528 | 781.22 | 864 | 1043.20 |
| (Day 9) | | (Day 23) | | (Day 37) | |

(ac) Patient 030
Date of Birth: 19.12.1944
Dose: 950 mg
Sex: Male

| | | | Con- | | Con- |
|---|---|---|---|---|---|
| Time | Concentration | Time | centration | Time | centration |
| (hr) | (μM) | (hr) | (μM) | (hr) | (μM) |
| 0 | n.d. | 336 | | 672 | |
| | | (Day 15) | | (Day 29) | |
| 2 | 116.56 | 338 | | 674 | |
| 3 | 219.04 | 339 | | 675 | |
| 4 | 332.61 | 340 | | 676 | |
| 24 | 214.26 | 360 | | 696 | |
| (Day 2) | | (Day 16) | | (Day 30) | |
| 168 | | 504 | | 840 | |
| (Day 8) | | (Day 22) | | (Day 36) | |
| 170 | | 506 | | 842 | |
| 171 | | 507 | | 843 | |
| 172 | | 508 | | 844 | |
| 192 | | 528 | | 864 | |
| (Day 9) | | (Day 23) | | (Day 37) | |

(ad) Patient 031
Date of Birth: 13.11.1952
Dose: 950 mg
Sex: Male

| | | | Con- | | Con- |
|---|---|---|---|---|---|
| Time | Concentration | Time | centration | Time | centration |
| (hr) | (μM) | (hr) | (μM) | (hr) | (μM) |
| 0 | n.d. | 336 | 554.28 | 672 | 812.71 |
| | | (Day 15) | | (Day 29) | |
| 2 | 266.82 | 338 | 668.41 | 674 | 920.63 |
| 3 | 337.51 | 339 | 715.03 | 675 | 882.04 |
| 4 | 335.80 | 340 | 697.4 | 676 | 938.32 |
| 24 | 347.52 | 360 | 799.77 | 696 | 1031.14 |
| (Day 2) | | (Day 16) | | (Day 30) | |
| 168 | 237.01 | 504 | 754.63 | 840 | |
| (Day 8) | | (Day 22) | | (Day 36) | |
| 170 | 512.93 | 506 | 794.18 | 842 | |
| 171 | 463.15 | 507 | 861.48 | 843 | |
| 172 | 426.87 | 508 | 998.35 | 844 | |
| 192 | 515.81 | 528 | 958.09 | 864 | |
| (Day 9) | | (Day 23) | | (Day 37) | |

(ae) Patient 032
Date of Birth: 17.09.1935
Dose: 950 mg
Sex: Male

| | | | Con- | | Con- |
|---|---|---|---|---|---|
| Time | Concentration | Time | centration | Time | centration |
| (hr) | (μM) | (hr) | (μM) | (hr) | (μM) |
| 0 | BLQ | 336 | | 672 | |
| | | (Day 15) | | (Day 29) | |
| 2 | 291.69 | 338 | | 674 | |
| 3 | 191.19 | 339 | | 675 | |
| 4 | 231.38 | 340 | | 676 | |
| 24 | 160.60 | 360 | | 696 | |
| (Day 2) | | (Day 16) | | (Day 30) | |
| 168 | 192.28 | 504 | | 840 | |
| (Day 8) | | (Day 22) | | (Day 36) | |
| 170 | 439.06 | 506 | | 842 | |
| 171 | 478.03 | 507 | | 843 | |
| 172 | 516.70 | 508 | | 844 | |
| 192 | 495.07 | 528 | | 864 | |
| (Day 9) | | (Day 23) | | (Day 37) | |

(af) Patient 033
Date of Birth: 19.08.1936
Dose: 1200 mg
Sex: Male

| | Concen- | | Concen- | | |
|---|---|---|---|---|---|
| Time | tration | Time | tration | Time | Concentration |
| (hr) | (μM) | (hr) | (μM) | (hr) | (μM) |
| 0 | 0.00 | 336 | 637.00 | 672 | 1019.28 |
| | | (Day 15) | | (Day 29) | |
| 2 | 441.43 | 338 | 925.60 | 674 | 1231.36 |
| 3 | 395.64 | 339 | 840.74 | 675 | 1112.56 |
| 4 | 373.92 | 340 | 816.70 | 676 | 1096.93 |
| 24 | 371.41 | 360 | 915.01 | 696 | 1105.61 |
| (Day 2) | | (Day 16) | | (Day 30) | |
| 168 | 317.41 | 504 | 713.28 | 840 | 1040.57 |
| (Day 8) | | (Day 22) | | (Day 36) | |
| 170 | 734.84 | 506 | 1147.58 | 842 | 1114.49 |
| 171 | 657.48 | 507 | 1061.41 | 843 | 1226.74 |
| 172 | 637.11 | 508 | 1007.50 | 844 | 1204.86 |
| 192 | 590.63 | 528 | 1172.58 | 864 | 1317.84 |
| (Day 9) | | (Day 23) | | (Day 37) | |

(ag) Patient 034
Date of Birth: 15.04.1948
Dose: 1200 mg
Sex: Female

| | Concen- | | Concen- | | |
|---|---|---|---|---|---|
| Time | tration | Time | tration | Time | Concentration |
| (hr) | (μM) | (hr) | (μM) | (hr) | (μM) |
| 0 | n.d | 336 | 644.35 | 672 | 1079.09 |
| | | (Day 15) | | (Day 29) | |
| 2 | 440.36 | 338 | 1026.11 | 674 | 1308.93 |
| 3 | 401.05 | 339 | 1115.82 | 675 | 1448.79 |
| 4 | 559.64 | 340 | 954.51 | 676 | 1383.21 |
| 24 | 409.45 | 360 | 1024.33 | 696 | 1307.12 |
| (Day 2) | | (Day 16) | | (Day 30) | |
| 168 | 350.51 | 504 | 923.50 | 840 | |
| (Day 8) | | (Day 22) | | (Day 36) | |
| 170 | 893.14 | 506 | 1354.14 | 842 | |
| 171 | 770.66 | 507 | 1217.57 | 843 | |
| 172 | 729.99 | 508 | 1440.82 | 844 | |
| 192 | 721.75 | 528 | 1363.80 | 864 | |
| (Day 9) | | (Day 23) | | (Day 37) | |

(ah) Patient 035
Date of Birth: 28.08.1958
Dose: 1200 mg
Sex: Male

| | Concen- | | Concen- | | |
|---|---|---|---|---|---|
| Time | tration | Time | tration | Time | Concentration |
| (hr) | (μM) | (hr) | (μM) | (hr) | (μM) |
| 0 | n.d | 336 | 476.33 | 672 | |
| | | (Day 15) | | (Day 29) | |
| 2 | 298.06 | 338 | 570.40 | 674 | |
| 3 | 196.36 | 339 | 621.24 | 675 | |
| 4 | 316.74 | 340 | 649.3 | 676 | |
| 24 | 261.19 | 360 | 704.40 | 696 | |
| (Day 2) | | (Day 16) | | (Day 30) | |
| 168 | 254.36 | 504 | 599.62 | 840 | |
| (Day 8) | | (Day 22) | | (Day 36) | |
| 170 | 459.98 | 506 | 774.20 | 842 | |
| 171 | 478.99 | 507 | 925.12 | 843 | |
| 172 | 592.29 | 508 | 1022.64 | 844 | |
| 192 | 542.94 | 528 | 1172.95 | 864 | |
| (Day 9) | | (Day 23) | | (Day 37) | |

(al) Patient 040
Date of Birth: 20.06.1952
Dose: 1000 mg
Sex: Male

| | Con- | | | | Con- |
|---|---|---|---|---|---|
| Time | centration | Time | Concentration | Time | centration |
| (hr) | (μM) | (hr) | (μM) | (hr) | (μM) |
| 0 | n.d. | 336 | 305.35 | 672 | 489.94 |
| | | (Day 15) | | (Day 29) | |
| 2 | 187.66 | 338 | 487.42 | 674 | 558.12 |
| 3 | 167.62 | 339 | 426.74 | 675 | 532.65 |
| 4 | 189.71 | 340 | 476.48 | 676 | 607.14 |
| 24 | 148.06 | 360 | 484.91 | 696 | 697.26 |
| (Day 2) | | (Day 16) | | (Day 30) | |
| 168 | 119.66 | 504 | 353.16 | 840 | 573.56 |
| (Day 8) | | (Day 22) | | (Day 36) | |
| 170 | 367.81 | 506 | 501.95 | 842 | 625.73 |
| 171 | 343.25 | 507 | 554.18 | 843 | 747.73 |
| 172 | 325.68 | 508 | 515.30 | 844 | No sample |
| 192 | 272.17 | 528 | 524.78 | 864 | 826.44 |
| (Day 9) | | (Day 23) | | (Day 37) | |

(am) Patient 041
Date of Birth: 23.05.1945
Dose: 1000 mg
Sex: Male

| | Con- | | | | Con- |
|---|---|---|---|---|---|
| Time | centration | Time | Concentration | Time | centration |
| (hr) | (μM) | (hr) | (μM) | (hr) | (μM) |
| 0 | n.d. | 336 | 294.39 | 672 | 670.13 |
| | | (Day 15) | | (Day 29) | |
| 2 | 222.94 | 338 | 411.42 | 674 | 546.09 |
| 3 | 232.54 | 339 | 485.07 | 675 | 739.71 |
| 4 | 207.41 | 340 | 478.48 | 676 | 802.50 |
| 24 | 175.01 | 360 | 508.44 | 696 | 785.67 |
| (Day 2) | | (Day 16) | | (Day 30) | |
| 168 | 171.34 | 504 | 558.23 | 840 | 714.83 |
| (Day 8) | | (Day 22) | | (Day 36) | |
| 170 | 342.33 | 506 | 674.45 | 842 | 737.74 |
| 171 | 412.52 | 507 | 748.03 | 843 | 1006.29 |
| 172 | 357.46 | 508 | 676.27 | 844 | No sample |
| 192 | 395.56 | 528 | 703.40 | 864 | 1209.31 |
| (Day 9) | | (Day 23) | | (Day 37) | |

(an) Patient 042
Date of Birth: 21.02.1947
Dose: 1000 mg
Sex: Female

| | Con- | | | | Con- |
|---|---|---|---|---|---|
| Time | centration | Time | Concentration | Time | centration |
| (hr) | (μM) | (hr) | (μM) | (hr) | (μM) |
| 0 | n.d. | 336 | 585.96 | 672 | 1036.40 |
| | | (Day 15) | | (Day 29) | |
| 2 | 271.56 | 338 | 645.49 | 674 | 1178.80 |
| 3 | 358.73 | 339 | 675.08 | 675 | 1191.64 |
| 4 | 281.49 | 340 | 636.03 | 676 | No sample |
| 24 | 238.71 | 360 | 650.70 | 696 | 1281.13 |
| (Day 2) | | (Day 16) | | (Day 30) | |
| 168 | 250.60 | 504 | 764.91 | 840 | 1248.6 |
| (Day 8) | | (Day 22) | | (Day 36) | |
| 170 | 391.37 | 506 | 871.78 | 842 | 1140.5 |
| 171 | 461.28 | 507 | 951.85 | 843 | 1177.0 |
| 172 | 435.82 | 508 | 986.97 | 844 | 1158.6 |
| 192 | 501.59 | 528 | 1231.51 | 864 | 1251.9 |
| (Day 9) | | (Day 23) | | (Day 37) | |

(ai) Patient 036
Date of Birth: 23.03.1946
Dose: 1200 mg
Sex: Female

| | Concen- | | Concen- | | |
|---|---|---|---|---|---|
| Time | tration | Time | tration | Time | Concentration |
| (hr) | (μM) | (hr) | (μM) | (hr) | (μM) |
| 0 | n.d. | 336 | 556.91 | 672 | No sample |
| | | (Day 15) | | (Day 29) | |
| 2 | 442.58 | 338 | 840.43 | 674 | No sample |
| 3 | 431.11 | 339 | 865.85 | 675 | No sample |
| 4 | 360.54 | 340 | 930.0 | 676 | No sample |
| 24 | 708.42 | 360 | 657.31 | 696 | No sample |
| (Day 2) | | (Day 16) | | (Day 30) | |
| 168 | 285.95 | 504 | 968.95 | 840 | 947.50 |
| (Day 8) | | (Day 22) | | (Day 36) | |
| 170 | 679.68 | 506 | 1143.19 | 842 | 1247.49 |
| 171 | 678.01 | 507 | 1131.52 | 843 | 1293.03 |
| 172 | 665.00 | 508 | 1117.46 | 844 | 1234.17 |
| 192 | 295.33 | 528 | No sample | 864 | 1063.33 |
| (Day 9) | | (Day 23) | | (Day 37) | |

(aj) Patient 037
Date of Birth: 19.04.1958
Dose: 1200 mg
Sex: Female

| | Con- | | Concen- | | |
|---|---|---|---|---|---|
| Time | centration | Time | tration | Time | Concentration |
| (hr) | (μM) | (hr) | (μM) | (hr) | (μM) |
| 0 | n.d. | 336 | 779.78 | 672 | 1316.96 |
| | | (Day 15) | | (Day 29) | |
| 2 | 255.87 | 338 | 1053.68 | 674 | 1457.82 |
| 3 | 302.40 | 339 | 1044.19 | 675 | 1456.41 |
| 4 | 278.36 | 340 | 1025.26 | 676 | 1451.35 |
| 24 | 418.44 | 360 | 1135.41 | 696 | 1530.33 |
| (Day 2) | | (Day 16) | | (Day 30) | |
| 168 | 422.38 | 504 | 1104.32 | 840 | |
| (Day 8) | | (Day 22) | | (Day 36) | |
| 170 | 633.83 | 506 | 1199.98 | 842 | |
| 171 | 659.95 | 507 | 1223.32 | 843 | |
| 172 | 711.66 | 508 | 1393.91 | 844 | |
| 192 | 841.24 | 528 | 1387.00 | 864 | |
| (Day 9) | | (Day 23) | | (Day 37) | |

(ak) Patient 038
Date of Birth: 06.10.1957
Dose: 1200 mg
Sex: Female

| | Concen- | | Con- | | |
|---|---|---|---|---|---|
| Time | tration | Time | centration | Time | Concentration |
| (hr) | (μM) | (hr) | (μM) | (hr) | (μM) |
| 0 | n.d. | 336 | 461.60 | 672 | 681.90 |
| | | (Day 15) | | (Day 29) | |
| 2 | 312.62 | 338 | 808.36 | 674 | 931.50 |
| 3 | 314.43 | 339 | 682.38 | 675 | 884.53 |
| 4 | 297.98 | 340 | 672.23 | 676 | 708.47 |
| 24 | 241.95 | 360 | 749.49 | 696 | 802.27 |
| (Day 2) | | (Day 16) | | (Day 30) | |
| 168 | 268.04 | 504 | 566.42 | 840 | 687.10 |
| (Day 8) | | (Day 22) | | (Day 36) | |
| 170 | 502.09 | 506 | 658.21 | 842 | 895.36 |
| 171 | 480.58 | 507 | 787.75 | 843 | 954.75 |
| 172 | 538.47 | 508 | 698.94 | 844 | 913.67 |
| 192 | 492.87 | 528 | 746.12 | 864 | 958.10 |
| (Day 9) | | (Day 23) | | (Day 37) | |

n.s—No sample;

### Pharmacokinetic Summary

The half life of CXR1002 is extremely long and could not be defined during the period of evaluation (6 weeks) (FIG. 10). CXR1002 accumulates in the blood following each weekly dose. This is exemplified in FIG. 11, which shows accumulating plasma levels after 6 weekly 50 mg doses in patient 01-004. There is greater exposure with increasing dose of CXR1002 and with increasing duration of treatment (FIG. 12). The maximal blood level reached was 617 μM.

### Efficacy

The best response to CXR1002 treatment was stable disease lasting 7 months. Four patients had stable disease ≧4 months (range 20 to 35 weeks) (Table 12). Of these, 1 patient diagnosed with pancreatic cancer had radiographic evidence of tumour shrinkage which did not meet the criteria of partial response.

TABLE 12
Patients with Stable Disease (SD) >4 months on Study CXR1002-001

| | | | On-Study | |
|---|---|---|---|---|
| Dose | | Best | Duration | Number Prior |
| (mg/week) | Tumour Type | response | (weeks) | Treatments |
| 50 | pancreatic | SD | 35 | 2 |
| 100 | colorectal | SD | 20 | 3 |
| 200 | cervical | SD | 22* | 2 |
| 200 | anaplastic | SD | 22* | 3 |
| | thyroid | | | |

*Patient remains on study

### Example 6

### Combinations of CXR1002 with Other Drugs

The aim of this study was to combine CXR1002 with other agents to ascertain whether an enhanced response to the combination of drugs was observed.

The results as presented are from a single assay in which the cell lines listed in Table 13 below were exposed to CXR1002 or the test items listed in Table 14 or the test items in combination with CXR1002.

FIG. 28 shows a tabulated summary of the results taken from the individual graphs of the cytotoxicity assays on individual cell lines 2 (black curve—test item alone; blue curve—test item+a single dose of CXR1002). Green indicates that the cells were more sensitive to a combination of test item and CXR1002 than to the test item alone. Yellow indicates that there was no apparent change in sensitivity and therefore no further analyses is suggested. Red indicates a possible adverse effect of the combination of drug with CXR1002. The full data is shown in FIGS. 29-60.

### Methods

The cell lines were purchased from The American Type Culture Collection (ATCC) via LGC Promochem (London, UK), the European Collection of Cell Cultures (ECACC) via Sigma-Aldrich, UK, or the Health Science Research Resources Bank of the Japan Health Science Foundation (JHSF): (Refer to Table 13). Cell line H was supplied by the Biomedical Research Centre, Ninewells Hospital, Dundee.

TABLE 13
Cell lines purchased from commercial suppliers and stored at CXR
Biosciences:

| Cell Line | Tissue | Supplier | Product Code |
|---|---|---|---|
| OUMS-27 | Sarcoma | JHSF | IFO50488 |
| SW1353 | Sarcoma | ATCC | HTB-94 |
| PANC1 | Pancreas | ATCC | CRL-1469 |
| BxPc3 | Pancreas | ATCC | CRL-1687 |
| HPAFII | Pancreas | ATCC | CRL-1997 |
| Capan2 | Pancreas | ATCC | HTB-80 |
| SK-OV3 | Ovary | ATCC | HTB-77 |
| TOV-21G | Ovary | ATCC | CRL-11730 |
| OV-90 | Ovary | ATCC | CRL-11732 |
| OVCAR3 | Ovary | ATCC | HTB-161 |
| PC3 | Prostate | ECACC | 90112714 |
| MDA-MB-157 | Breast | ECACC | 92020422 |
| CACO2 | Colon | ECACC | 86010202 |
| HepG2 | Liver | ECACC | 85011430 |

TABLE 14
Test Item Supplier Details

| Test Item | Supplier | Catalogue Ref. |
|---|---|---|
| Doxorubicin hydrochloride | Sigma | D1515 |
| Gemcitabine | Sequoia | SRP01265g |
| Cisplatin | Sigma | P4394 |
| Docetaxel | Fluka | 01885 |
| 5-FU | Sigma | F6627 |
| Roscovitine | Sigma | R7772 |
| DPQ | Sigma | D5314 |
| Geldanamycin | Apollo scientific | BIG2461 |
| Rapamycin | Apollo scientific | BIR8101 |
| LY294002 | Sigma | L9908 |
| U0126 | Merck | 662005 |

Test compounds were dissolved in DMSO to make stock solutions of an appropriate concentration. The stock solutions were further diluted in DMSO to produce additional stock solutions as necessary. The amount of DMSO added to the medium was 1% of the final volume.

Cells were plated at the optimal plating density for that cell line in 96-well plates and allowed to attach overnight. The next day, the medium was removed and replaced with fresh medium containing the dose ranges of test items. The cells were exposed to 5-FU, cisplatin, docetaxel, doxorubicin, geldanamycin, gemcitabine, rapamycin or roscovitine in Roswell Park Memorial Institute (RPMI) medium containing 10% Foetal Calf Serum (FCS) and 2 mM Glutamine at 37° C. and 5% CO₂ for 48 hours. The concentrations of CXR1002 or other agents to which the cells were exposed were as previously determined or as suggested by relevant literature (see Table 14 below). There were 3 replicates for each test item concentration.

TABLE 15
Final concentrations of compounds in tissue culture medium.

| | |
|---|---|
| Compound | Final Concentration μM |

| | | | | |
|---|---|---|---|---|
| 5-Fluorouracil | 1 | 10 | 100 | 300 |
| Cisplatin | 1 | 10 | 100 | 300 |
| CXR1002 | 100 | 300 | 500 | 1000 |
| Docetaxel | 0.1 | 1 | 10 | 100 |
| Doxorubicin | 0.01 | 0.1 | 1 | 10 |
| Geldanamycin | 0.0001 | 0.001 | 0.1 | 1 |
| Gemcitabine | 1 | 10 | 100 | 300 |
| Rapamycin | 0.0001 | 0.001 | 0.1 | 1 |
| Roscovitine | 1 | 3 | 10 | 50 |

From the results of the single compound assays, appropriate doses ranges were determined for use in the combinatorial assays with CXR 1002. These are shown in Table 16:

TABLE 16
Final concentrations of compounds for cytotoxicity assays in combination with CXR1002.

| Cell Line | [1002]μM | [Doxorubicin]μM | | | | [Gemcitabine]μM | | | | [Cisplatin]μM | | | |
|---|---|---|---|---|---|---|---|---|---|---|---|---|---|
| H | 300 | 0.001 | 0.01 | 0.1 | 1 | 0.01 | 0.1 | 1 | 100 | 0.1 | 1 | 10 | 30 |
| OUMS-27 | 300 | 0.01 | 0.1 | 0.5 | 5 | 0.01 | 0.1 | 1 | 100 | 1 | 3 | 5 | 10 |
| SW1353 | 300 | 0.01 | 0.1 | 0.2 | 0.5 | 1 | 10 | 100 | 300 | 10 | 20 | 30 | 50 |
| PANC1 | 300 | 0.01 | 0.1 | 0.5 | 1 | 0.01 | 0.1 | 1 | 100 | 1 | 10 | 30 | 50 |
| BxPc3 | 150 | 0.01 | 0.1 | 0.5 | 1 | 0.01 | 0.1 | 1 | 100 | 1 | 10 | 20 | 30 |
| HPAFII | 300 | 0.01 | 0.1 | 0.5 | 5 | 0.01 | 0.1 | 1 | 100 | 10 | 20 | 50 | 100 |
| Capan2 | 300 | 0.1 | 1 | 3 | 5 | 0.1 | 1 | 10 | 100 | 10 | 100 | 150 | 200 |
| SK-OV3 | 300 | 0.001 | 0.01 | 0.1 | 1 | 0.001 | 0.01 | 0.1 | 1 | 1 | 10 | 30 | 50 |
| TOV-21G | 300 | 0.01 | 0.05 | 0.1 | 0.25 | 0.01 | 0.1 | 1 | 50 | 1 | 10 | 15 | 30 |
| OV-90 | 300 | 0.1 | 0.3 | 1 | 10 | 0.1 | 1 | 10 | 100 | 10 | 30 | 100 | 300 |
| OVCAR-3 | 300 | 0.01 | 0.03 | 0.1 | 0.3 | 0.01 | 0.1 | 1 | 50 | 0.1 | 1 | 10 | 20 |
| PC3 | 300 | 0.01 | 0.1 | 1 | 10 | 0.1 | 1 | 10 | 100 | 1 | 10 | 100 | 300 |
| CACO2 | 300 | 0.1 | 0.5 | 1 | 5 | 0.1 | 1 | 10 | 100 | 10 | 20 | 30 | 50 |
| MDA-MB-157 | 300 | 0.1 | 0.3 | 0.5 | 2 | 1 | 10 | 100 | 300 | 10 | 100 | 200 | 300 |
| HepG2 | 100 | 0.01 | 0.1 | 0.2 | 0.5 | 0.01 | 0.1 | 1 | 10 | 10 | 20 | 30 | 50 |

| Cell Line | [1002]μM | [Docetaxel]μM | | | | [5FU]μM | | | | [Roscovitine]μM | | | |
|---|---|---|---|---|---|---|---|---|---|---|---|---|---|
| H | 300 | 0.01 | 0.1 | 1 | 10 | 1 | 10 | 100 | 300 | 3 | 10 | 20 | 30 |
| OUMS-27 | 300 | 0.01 | 0.1 | 1 | 10 | 1 | 10 | 100 | 300 | 3 | 10 | 20 | 30 |
| SW1353 | 300 | 0.001 | 0.01 | 0.1 | 1 | 1 | 10 | 100 | 300 | 1 | 5 | 10 | 50 |
| PANC1 | 300 | 0.001 | 0.01 | 0.1 | 1 | 1 | 10 | 100 | 300 | 1 | 5 | 10 | 50 |
| BxPc3 | 150 | 0.001 | 0.01 | 0.1 | 1 | 1 | 10 | 100 | 300 | 1 | 5 | 10 | 20 |
| HPAFII | 300 | 0.01 | 0.1 | 1 | 100 | 1 | 10 | 100 | 300 | 1 | 5 | 10 | 50 |
| Capan2 | 300 | 0.1 | 1 | 10 | 100 | 1 | 10 | 100 | 300 | 3 | 10 | 30 | 50 |
| SK-OV3 | 300 | 0.001 | 0.01 | 0.1 | 1 | 1 | 10 | 100 | 300 | 3 | 10 | 30 | 50 |
| TOV-21G | 300 | 0.01 | 0.1 | 1 | 10 | 1 | 10 | 100 | 300 | 1 | 5 | 10 | 20 |
| OV-90 | 300 | 0.01 | 0.1 | 1 | 100 | 1 | 10 | 100 | 300 | 3 | 10 | 30 | 50 |
| OVCAR-3 | 300 | 0.01 | 0.1 | 1 | 10 | 0.1 | 1 | 10 | 100 | 1 | 3 | 10 | 20 |
| PC3 | 300 | 0.1 | 1 | 10 | 100 | 1 | 10 | 100 | 300 | 1 | 3 | 10 | 50 |
| CACO2 | 300 | 0.01 | 0.1 | 1 | 10 | 1 | 10 | 100 | 300 | 1 | 3 | 10 | 30 |
| MDA-MB-157 | 300 | 0.001 | 0.01 | 0.1 | 1 | 1 | 10 | 100 | 300 | 1 | 3 | 10 | 30 |
| HepG2 | 100 | 0.001 | 0.01 | 0.1 | 1 | 1 | 10 | 100 | 300 | 1 | 3 | 10 | 30 |

| | [1002]μM | [Geldanamycin]μM | | | | [Rapamycin]μM | | | |
|---|---|---|---|---|---|---|---|---|---|
| H | 300 | 0.001 | 0.01 | 0.1 | 0.5 | 0.001 | 0.1 | 1 | 10 |
| OUMS-27 | 300 | 0.001 | 0.01 | 0.1 | 0.5 | 0.001 | 0.1 | 1 | 10 |
| SW1353 | 300 | 0.001 | 0.01 | 0.1 | 1 | 0.001 | 0.1 | 1 | 10 |
| PANC1 | 300 | 0.1 | 1 | 5 | 10 | 0.001 | 0.1 | 1 | 10 |
| BxPc3 | 150 | 0.001 | 0.01 | 0.1 | 0.5 | 0.0001 | 0.001 | 0.01 | 0.1 |
| HPAFII | 300 | 0.1 | 1 | 5 | 10 | 0.001 | 0.1 | 1 | 10 |
| Capan2 | 300 | 0.1 | 1 | 5 | 10 | 0.001 | 0.1 | 1 | 10 |
| SK-OV3 | 300 | 0.1 | 1 | 5 | 10 | 0.001 | 0.1 | 1 | 10 |
| TOV-21G | 300 | 0.001 | 0.01 | 0.1 | 1 | 0.0001 | 0.001 | 0.01 | 0.1 |
| OV-90 | 300 | 0.001 | 0.01 | 0.1 | 1 | 0.001 | 0.1 | 1 | 10 |
| OVCAR-3 | 300 | 0.001 | 0.01 | 0.1 | 1 | 0.0001 | 0.001 | 0.01 | 0.1 |
| PC3 | 300 | 0.1 | 1 | 5 | 10 | 0.0001 | 0.001 | 0.01 | 0.1 |
| CACO2 | 300 | 0.001 | 0.01 | 0.1 | 1 | 0.0001 | 0.001 | 0.01 | 0.1 |
| MDA-MB-157 | 300 | 0.1 | 1 | 5 | 10 | 0.0001 | 0.001 | 0.01 | 0.1 |
| HepG2 | 100 | 0.001 | 0.01 | 0.1 | 0.5 | 0.001 | 0.1 | 1 | 10 |

Notes:
HepG2 and BxPc3 cells are more sensitive to treatment with CXR1002 than the other lines used in these assays. As a result the single dose of CXR1002 used for HepG2 cells in the combination assays as 100 μM and for BxPc3 cells this was 150 μM.

Following exposure to Test Items, the CellTitre-Glo Luminescent Cell Viability Assay to measure ATP content was performed according to the manufacturer's detailed instructions (Promega Corporation, Technical Bulletin No. 288, and Cell notes, Issue 10, 2004).

The results of the ATP depletion assay were corrected for background luminescence and expressed as a percentage of the vehicle control value using Microsoft Excel software. Point-to-point spline analysis was performed and the results graphed in GraphPad Prism as cell viability (percentage of vehicle control) versus Test Item concentration.

### Conclusions

Doxorubicin, gemcitabine, geldanamycin and roscovitine were shown to increase the sensitivity of a number of the cell lines. Rapamycin increased sensitivity in the four ovarian lines tested and in HepG2 cells. Interestingly from the clinical perspective, when MDA-MB-157 (breast) cells were treated with the combination of 5-FU, a drug used in the treatment of breast cancer, and CXR1002, there was an apparent increase in sensitivity.

TABLE 17
Summary of Conclusions

| | | Drugs for repeat cytoxicity assays in |
|---|---|---|
| Cell Line | Tissue | combination with CXR1002 |
| Panc1 | Pancreas | Doxorubicin, Gemcitabine, |
| | | Geldanamycin |
| BxPc3 | Pancreas | Gemcitabine |
| Capan2 | Pancreas | Roscovitine |
| H | Chondrosarcoma | Gemcitabine |
| SK-OV3 | Ovary | Rapamycin |
| TOV-21G | Ovary | Gemcitabine, 5-FU |
| OV-90 | Ovary | Doxorubicin, Geldanamycin, |
| | | Rapamycin, Roscovitine |
| OVCAR-3 | Ovary | Gemcitabine |
| PC3 | Prostate | Doxorubicin, Geldanamycin, |
| | | Roscovitine |
| MDA-MB-157 | Breast | 5-FU |
| HepG2 | Liver | Gemcitabine, Geldanamycin, |
| | | Rapamycin, Roscovitine |

### Example 7

### Further Combination Data

The objective of this study is to combine CXR1002 with known anti-cancer agents, both investigational and marketed drugs, in an effort to achieve enhanced tumour cell killing ie. to potentiate mode of action.

In a 48 hour cytotoxicity assay the following compounds were tested at fixed concentrations, derived from a review of the literature, in the presence of CXR1002 (0-1 mM):

1. MAP kinase inhibitor (MEK1/2) (compound name, UO126)
2. AKT/PI3K inhibitor (compound name, LY294002)
3. PARP inhibitor (compound name, DPQ)

TABLE 18
Cell signalling inhibitors used in this study

| | | Concentration | Concentration | | | |
|---|---|---|---|---|---|---|
| Chemical | Indication | (literature) | used in this study | Duration | Mechanism | Ref. |
| U0126 | Breast | i) 6, 12.5, | 10 μM | Up to 10 h, | MEK1/2 | Mol. Cancer |
| | cancer cell | or 50 μM | | activity may | inhibitor | Ther., |
| | lines | ii) IC₅₀: | | decline after | | 303-309, |
| | | 10-20 μM | | longer incubation | | (2002) |
| LY294002 | Pancreatic | 10-75 μM | 12.5 μM | | Akt/PI3K | J. Exp. |
| | cell lines | IC₅₀: 50 μM, | | | inhibitor | & Clin. |
| | (+cisplatin) | 10-25 μM | | | | Res., |
| | | At 50 μM no | | | | (2008) |
| | | toxic effect | | | | |
| DPQ | | 20-30 μM | 20 μM | | PARP | Anticancer |
| | | | | | inhibitor | Drug |
| | | | | | | Designs., |
| | | | | | | 107 (1991) |

### Method

The cell lines were purchased from The American Type Culture Collection (ATCC) via LGC Promochem (London, UK), the European Collection of Cell Cultures (ECACC) via Sigma-Aldrich, UK, or the Health Science Research Resources Bank of the Japan Health Science Foundation (JHSF): (Refer to Table 19). Sarcoma cell line H was supplied by the Biomedical Research Centre, Ninewells Hospital, Dundee.

TABLE 19
Cell lines purchased from commercial suppliers and stored at CXR
Biosciences:

| Cell Line | Tissue | Supplier | Product Code |
|---|---|---|---|
| OUMS-27 | Sarcoma | JHSF | IFO50488 |
| SW1353 | Sarcoma | ATCC | HTB-94 |
| PANC-1 | Pancreas | ATCC | CRL-1469 |
| BxPc3 | Pancreas | ATCC | CRL-1687 |
| HPAFII | Pancreas | ATCC | CRL-1997 |
| Capan2 | Pancreas | ATCC | HTB-80 |
| SK-OV3 | Ovary | ATCC | HTB-77 |
| TOV-21G | Ovary | ATCC | CRL-11730 |
| OV-90 | Ovary | ATCC | CRL-11732 |
| OVCAR3 | Ovary | ATCC | HTB-161 |
| PC3 | Prostate | ECACC | 90112714 |
| MDA-MB-157 | Breast | ECACC | 92020422 |
| CACO2 | Colon | ECACC | 86010202 |
| HepG2 | Liver | ECACC | 85011430 |

TABLE 20
Test Item Supplier Details

| Test Item | Supplier | Catalogue Ref. |
|---|---|---|
| U0126 | Sigma | U0126 |
| LY294002 | Sigma | L9908 |
| DPQ | Sigma | D5314 |

### Cell Culture

Test compounds were dissolved in DMSO to make stock solutions of an appropriate concentration. The stock solutions were further diluted in DMSO to produce additional stock solutions as necessary. The concentrations of the original stock solutions and the additional stock solutions will be recorded in the appropriate CXR Study folder and in the Study Report. The final amount of DMSO added to the medium was 1% of the final volume.

Cells were plated at the optimal plating density for that cell line in 96-well plates and allowed to attach overnight. The next day, cells were pre-treated with the inhibitors UO126 or LY294002 (see Tables 18 & 20) for 2 hrs, the medium was removed and replaced with fresh medium containing the appropriate dose of the test item. After 2 hrs, CXR1002 (concentration range 0-1 mM) together with the appropriate inhibitor was then added. Cells that were to be treated with DPQ received no pre-treatment. Cells were exposed to these compounds in Roswell Park Memorial Institute (RPMI) medium containing 10% Foetal Calf Serum (FCS) and 2 mM Glutamine at 37° C. and 5% CO₂ for 48 hours. There were 3 replicates for each test item concentration.

Following exposure to Test Items, the CellTitre-Glo Luminescent Cell Viability Assay to measure ATP content will be performed according to the manufacturer's detailed instructions (Promega Corporation, Technical Bulletin No. 288, and Cell notes, Issue 10, 2004).

The results of the ATP depletion assay were corrected for background luminescence and expressed as a percentage of the vehicle control value using Microsoft Excel software. The results were graphed as ATP content (percentage of appropriate control) versus Test Item concentration (CXR1002).

### Results

### UO126 (FIG. 61-64)

Use of the CXR1002/UO126 combination compared to CXR1002 alone revealed increased sensitivity in the following cell lines:

TABLE 21

| | | IC₅₀ | |
|---|---|---|---|
| Cell line | Tissue | CXR1002 (μM) | CXR1002 & U0126 (μM) |

| | | | |
|---|---|---|---|
| H | Sarcoma | 817 | 453 |
| OUMS-27 | Sarcoma | 821 | 410 |
| PANC-1 | Pancreas | >1000 | 897 |
| BxPc3 | Pancreas | 381 | 247 |
| TOV-21G | Ovarian | 615 | 389 |
| OV-90 | Ovarian | 714 | 329 |
| CaCO2 | Colon | 704 | 285 |

It is clear from this data that concomitant inhibition of MEK1/2 may enhance the efficacy of CXR1002 and that the effect may be selective to certain cell lines and therefore tumour types.

### LY294002 (FIG. 61-62)

LY294002 is a potent inhibitor of phosphoinositide 3-kinases. When used in conjunction with CXR1002 increased efficacy was noted in a select number of cell lines most notably the sarcoma cell line H.

### DPQ (FIG. 65-66)

Poly(ADP-ribose) polymerase-1 (PARP-1) is a nuclear enzyme involved in DNA repair, replication and cell cycle. However, its overactivation leads to nicotinamide adenine dinucleotide and ATP depletion and cell death. Use of the PARP inhibitor, DPQ, in conjunction with CXR1002 led to increased sensitivity in the following cell lines: HPAFII and Capan2 (pancreas) and SW1353 (sarcoma). Again, the synergistic response with the two drugs was selective.

### Conclusion

It is clear from this preliminary data that certain combinations of CXR1002 and the inhibitors UO126 or LY294002 or DPQ result in an increased level of cytotoxicity when compared to the use of CXR1002 alone. The exact mechanisms underpinning these observations remain to be elucidated. However, with regard to kinase pathways, the pathways (MAPK/PI3K/Akt) are known to form the core intracellular signalling routers in the stimulation of growth factors. Their expression, particularly that of PI3K/Akt, or that of their phosphorylated (activated) forms has been reported as a significant prognosis marker in sarcoma (Tomita, Y (2006)), gastric cancer (Cinti, C (2008)), pancreatic cancer (Chada, K S (2006)) and breast cancer (Park, S S (2007)). Therefore, inactivation of the PI3K/Akt or MAPK pathways should be effective as a specific chemotherapy against malignant tumours because of lower expression of activated forms in the surrounding tissues. In addition, these pathways play an essential role as survival signal pathways when cancer cells are exposed to a cellular stress. CXR1002 may cause cellular stress e.g. oxidative stress and therefore may activate certain stress-related responses. Therefore, use of inhibitors of these pathways might be expected to enhance the degree of cytotoxicity in cancer cell lines exposed to CXR 1002 and perhaps even in vivo.

Poly(ADP-ribose)polymerase (PARP) or poly(ADP-ribose)synthase (PARS) has an essential role in facilitating DNA repair, controlling RNA transcription, mediating cell death, and regulating immune response. In various cancer models, PARP inhibitors have been shown to potentiate radiation and chemotherapy by increasing apoptosis of cancer cells, limiting tumour growth, decreasing metastasis, and prolonging the survival of tumour-bearing animals. Again, it appears that use of PARP inhibitors in conjunction with CXR1002 potentiates cytotoxicity.

### Example 8

### ER Stress Effects of CXR1002

Investigation into the ER stress effects of CXR1007 were conducted by looking at whether CXR1002 induces expression of ER stress-regulated proteins and then splicing of XBPI MRNA upon CXR1002 induced ER stress.

### Induction of Expression of ER Stress-Regulated Protein

Panc-1 (pancreatic tumour) cells were treated with vehicle control (lane 1), with 500 μM of CXR1002 for 4 h (lane 2), with 500 μM of CXR1002 for 1 day (lane 3), with 500 μM of CXR1002 for 2 days (lane 4), with 500 μM of CXR1002 for 3 days and with 500 μM of CXR1002 for 4 days.

Western blots were performed on the protein extracts (equivalent protein concentrations were loaded onto the gels). The antibodies used were for known ER stress regulated protein Bip/GRP78, CHOP/DDIT3, IRE1alpha, TRB3 (Tribbles3), cleaved PARP (marker of apoptosis), and tubulin (loading control) (see FIG. 71).

This showed that CXR1002 altered expression of ER stress-regulated proteins.

Splicing of XBP1 mRNA as an Indicator of ER Stress Inclusion

FIG. 72 shows the results of RT-PCR analysis of XBP1 mRNA splicing using RNA templates from CXR1002 treated cells. XBP1-u: unspliced form of XBPI; XBPI-s: spliced form of XBP1.

Panel (A) of FIG. 72 shows Panc-1 cells that were treated with CXR1002 for different time courses. 1. Control; 2. 500 μM/1 day; 3. 500 μM/2 days; 4. μM/3 days; 5. 500 μM/4 days; 6. μM/1 day; 7. 740 μM/2 days.

Panel (B) of FIG. 72 shows HepG2 cells that were treated with 300 μM of CXR1002 for different time courses 1. Control/1 day; 2. Control/2 days; 3. Control/4 days; 4. Tunicamycin for 24 h; 5. Tunicamycin for 6 h; 6. 300 μM/1 day; 7. 300 μM/2 days; 8. 300 μM/3 days.

Tunicamycin, 10 mg/mL. This is a control compound known to induce ER stress and XBP-1 splicing.

The RT-PCR analysis shows that XBP-1 splicing varies from predominately unspliced to spliced after treatment with CXR1002. XBP-1 is known to be spliced when ER stress is induced.

### Example 9

PIM kinase activity after CXR1002 exposure. PIM kinase inhibition has been investigated for each of PIM-1, PIM-2 and PIM-3 kinase molecules.

### PIM 1 (h)

The PIM-1 assay is performed using the Upstate IC₅₀ Profiler Express™ service. In a final reaction volume of 25 μl, human recombinant PIM-1 (5-10mU) is incubated with 8 mM MOPS pH 7.0, 0.2 mM EDTA, 100 μM KKRNRTLTV, 10 mM MgAcetate and [γ-³³P-ATP] (specific activity approx. 500 cpm/pmol, concentration as required). After incubation for 40 minutes at room temperature, the reaction is stopped by the addition of 3% phosphoric acid solution. 10 μl of the reaction is then spotted onto a P30 filtermat and washed three times for 5 minutes in 75 mM phosphoric acid and once in methanol prior to drying and scintillation counting.

### PIM-2 (h)

The PIM2 assay is performed using the Upstate IC₅₀ Profiler Express™ service. In a final reaction volume of 25 μl, human recombinant PIM-2 (5-10 mU) is incubated with 8 mM MOPS pH 7.0, 0.2 mM EDTA, 300 μM RSRHSSYPAGT, 10 mM MgAcetate and [?-³³P-ATP] (specific activity approx. 500 cpm/pmol, concentration as required). After incubation for 40 minutes at room temperature, the reaction is stopped by the addition of 3% phosphoric acid solution. 10 μl of the reaction is then spotted onto a P30 filtermat and washed three times for 5 minutes in 75 mM phosphoric acid and once in menthanol prior to drying and scintillation counting.

### PIM-3 (h)

The PIM-3 assay is performed using the Upstate IC₅₀ Profiler Express™ service. In a final reaction volume of 25 μl, human recombinant PIM-3 (5-10 mU) is incubated with 8 mM MOPS pH 7.0, 0.2 mM EDTA, 300 μM RSRHSSYPAGT, 10 mM MgAcetate and [?-³³P-ATP] (specific activity approx. 500 cpm/pmol, concentration as required). After incubation for 40 minutes at room temperature, the reaction is stopped by the addition of 3% phosphoric acid solution. 10 μl of the reaction is then spotted onto a P30 filtermat and washed three times for 5 minutes in 75 mM phosphoric acid and once in menthanol prior to drying and scintillation counting.

### Results

In each of the three PIM kinase assays, CXR 1002 shows inhibition of the kinase molecules.

| Kinase | EC₅₀ |
|---|---|
| PIM 1 | 40 |
| PIM2 | 170 |
| PIM3 | 240 |

### Example 10

CXR1002 pharmacokinetics. PK sampling (repeat dose) in CXR1002 clinical trial

The methodology of this clinical trial is detailed in Example 5.

Plasma was collected at regular intervals (2, 3, 4 and 24 hrs post dose) every week from fasted patients (fasted minimum of 1 hour pre and post dose) administered weekly doses of CXR1002

### Treatment (Cohort, Patients and Weekly Dose):

### Cohort 2: Patient 004 (50 mg)

### Cohort 3: Patients 005-007 (100 mg)

### Cohort 4: Patients 008-010 (200 mg)

### Cohort 5: Patients 011-014 (300 mg)

### Cohort 6: Patients 015-017 (450 mg)

### Cohort 7: Patients 018-024 (600 mg)

### Cohort 8: Patients 025-028 (750 mg)

### *Cohort 9: Patients 029-032 (950 mg)

### Cohort 10: Patients 033-037 (1200 mg)

(*pt. 031 was not dosed)

FIGS. 74-78 show the results of the repeat dosing in terms of CXR2002 plasma levels.

Urine was collected over a 24 hour duration post each weekly dose and CXR1002 levels were measured in the total sample. FIG. 79 shows the urinary excretion (μg) of CXR1002 in 6 patients at 6 time points. FIG. 80 shows that the urinary excretion of CXR1002 is reflected in the pharmacokinetic profile of patient 29 with high levels of urinary excretion.

### Results of Repeat Dose Pharmacokinetics:

As shown in FIGS. 74-78, CXR1002 plasma concentration was cumulative and increased with both dose and duration of dosing. There was demonstrable dose equivalence (FIG. 75). As shown in FIG. 79, urinary excretion of CXR1002 increases with multiple doses and the pharmokinetic profile of CXR1002 changes to reflect urinary excretion (FIG. 80).

### Example 11

### CXR1002 Effects on LDL and HDL

For detailed methodology, see Example 5.

### Sample Selection and Rationale

Patients 004-036 were included in the analysis (*patients 34, 35 & 38 were not available for analysis. CXR1002 was administered (at dose increments; n=3-6) daily for a 6-week period. Plasma was collected and analysed for PK & PD effects. Data was initially grouped by dose and then re-grouped by PK (peak plasma exposure on wk 6). PD data: Plasma samples=baseline vs. wk 6 (peak plasma). Comparable graphs were plotted whether grouped by dose or drug exposure.

### Data Analysis:

Individual patient raw data was captured and represented in graphs as % change from baseline (screening). Individual patient data (% baseline) was plotted in Prism and data was grouped according to PEAK plasma [CXR1002] at wk 6. Data represents either mean±SEM values or median, range+individual data points.

FIGS. 81 and 82 show the effect (% baseline) of 6 weeks of CXR1002 treatment on plasma High-density lipoprotein cholesterol (HDL-C) and Low-density lipoprotein cholesterol (LDL-C) levels respectively for patients grouped by peak plasma exposure.

The data suggests an effect of CXR1002 on LDL (i.e. lowering effect) but not HDL (i.e. CXR1002 lowers ‘bad’ cholesterol but ‘good’ cholesterol remains unchanged). This effect is entirely predicted from the animal data and suggests a possible use in patients with conditions such as high cholesterol and hyperlipidemia.

### REFERENCES

1. Abdellatif, A. G and Preat, V. 3, s.l.: Toxicology & Applied Pharmacology, 1991, Vol. 111. 530-537. The modulation of rat-liver carcinogenesis by perfluorooctanoic acid, a peroxisome proliferator.
2. Abdellatif, A. G, et al. 11, s.l.: Carcinogenesis, 1990, Vol. 11. 1899-1902. Peroxisome proliferation and modulation of rat liver carcinogenesis by 2,4-dichlorophenoxyacetic acid, 2,4,5-trichlorrophenoxyacetic acid, perfluorooctanoic acid and nafenopin.
3. Adam, M et al. Cancer Res 2006 66(7):3828-35. Targeting PIM kinases impairs survival of hematopoietic cells transformed by kinase inhibitor-sensitive and kinase inhibitor-resistant forms of Fms-like tyrosine kinase 3 and BCR/ABL.
4. Alexander, B. H, et al. s.l.: Occupational & Environmental Medicine, 2003, Vol. 60. 722-729. Mortality of employees of a perfluorooctanesulphonyl fluoride manufacturing facility.
5. Alexander, B. H. s.l.: USEPA Public Docket Ar-226-1030a018, 2001. Mortality study of workers employed at the 3M Cottge Grove facility.
6. Amaravadi, R and Thompson. C B. J Clin Invest 2005 115(10):2618-24. The survival kinases Akt and PIM as potential pharmacological targets.
7. Andersen, M. E, et al. 1, s.l.: Toxicological Sciences, 2008, Vol. 102.3-14. Perfluoroalkyl acids and related chemistries—Toxicokinetics and modes of action.
8. Beier, U H et al. Int J Oncol 2007 30(6):1381-7. Overexpression of PIM-1 in head and neck squamous cell carcinomas.
9. Biegel, L. B, et al. 1, s.l.: Toxicological Sciences, 2001, Vol. 60.44-55. Mechanisms of extrahepatic tumour induction by peroxisome proliferators in male CD rats,
10. Biegel, L. B, et al. s.l.: Toxicology & Applied Pharmacology, 1995, Vol. 134. 18-25. Effects of ammonium perfluorooctanoate on Leydig cell function: In vitro, in vivo, and ex vivo studies.
11. Bjork, J. A and Wallace, K. B. 1, s.l.: Toxicological Science, 2009, Vol. 111. 89-99. Structure-activity relationships and human relevance for perfluoralkyl acid-induced transcriptional activation of peroxisome proliferation in liver cell cultures.
12. Brault, L et al. Haematologica 2010 Feb. 9 epub. PIM serine/threonine kinases in pathogenesis and therapy of haematological malignancies and solid cancers.
13. Butenhoff, et al. s.l.: Toxicology, 2003, Vol. 196. 95-116. The reproductive toxicology of ammonium perfluorooctanoate (APFO) in the rat.
14. Butenhoff, J, et al. 1, s.l.: Toxicological Sciences, 2002, Vol. 69.244-257. Toxicity of ammonium perfluorooctanoate in male cynomolgus monkeys after oral dosing for 6 months.
15. Cattley, R. C, et al. s.l.: Regulatory & Toxicological Pharmacology, 1998, Vol. 27. 47-60. Do peroxisome proliferating compounds pose a hepatocarcinogenic hazard to humans.
16. Chambers, K. T, Weber, S. M and Corbett, J. A. s.l.: American Journal of Physiology, Endocrinology & Metabolism, 2007, Vol. 292. E1052-E1061. PGD2-stimulated beta-cell apoptosis is associated with prolonged UPR activation.
17. Chiang, W F et al. Int J Oral Maxillofac Surg 2006 35(8):740-5. Up-regulation of a serine-threonine kinase proto-oncogene PIM-1 in oral squamous cell carcinoma.
18. Choi, J Y; et al. J Otolaryngol Head Neck Surg 2010 39(1):28-34. Clinical significance of the expression of galectin-3 and PIM-1 in laryngeal squamous cell carcinoma.
19. Chen, W W et al. Mol Cancer Res 2005 3(8):443-51. PIM family kinases enhance tumour growth of prostate cancer cells.
20. Chen, J et al. Am J Pathol 2009, 175(10):400-11. Hypoxia-mediated up-regulation of PIM-1 contributes to solid tumour formation.
21. Chen, J L et al. Blood 2008, 111(3):1677-85. PIM-1 and PIM-2 kinases are required for efficient pre-B-cell transformation by v-Abl oncogene.
22. Cheung, C, et al. s.l.: Cancer Research, 2004, Vol. 64. 3849-3854. Diminished hepatocellular proliferation in mice humanized for the nuclear receptor peroxisome proliferator-activated receptor alpha.
23. Cheung, H. H, et al. 12, s.l.: Experimental Cell Research, 2006, Vol. 312. 2347-2357. Involvement of caspase-2 and caspase-9 in endoplasmic reticulum stress-induced apoptosis: a role for the IAPs.
24. Cibull, T L et al. J. Clin. Pathol. 2006 59(3): 285-8. Overexpression of PIM-1 during progression of prostatic adenocarcinoma.
25. Cohen, A M et al. Leuk Lymphoma 2004 45(5):951-5. Increased expression of hPIM-2 gene in human chronic lymphocytic leukemia and non-Hodgkin lymphoma.
26. Clegg, E. D, et al. 1, s.l.: Reproductive Toxicology, 1997, Vol. 11.107-121. Leydig cell hyperplasia and adenoma formation: mechanisms and relevance to humans.
27. Cook, J. C, et al. s.l.: Critical Reviews in Toxicology, 1999, Vol. 29.169-261. Rodent leydig cell tumourigenesis: A review of the physiology, pathology, mechanisms, and relevance to humans.
28. Cook, J. C, et al. s.l.: Toxicology & Applied Pharmacology, 1992, Vol. 113. 209-217. Induction of Leydig cell adenomas by ammonium perfluorooctanoate: A possible endocrine related mechanism.
29. Corton, J, et al. s.l.: Biochimie, 1997, Vol. 79. 151-162. Peroxisome proliferators alter the expression of estrogen-metabolising enzymes.
30. Corton, J. C., Anderson, S. P and Stauber, A. s.l.: Annual Review of Pharmacology and Toxicology, 2000, Vol. 40. 491-518. Central role of peroxisome proliferator-activated receptors in the actions of peroxisome proliferators.
31. Dai, H, et al. Prostate 2005, 65(3):276-86. PIM-2 upregulation: biological implications associated with disease progression and perineueral invasion in prostate cancer.
32. DeWitt, J. C, et al. 1, s.l.: Critical Reviews in Toxicology, 2009, Vol. 39.76-94. Immunotoxicity of perfluorooctanoic acid and perfluorooctane sulfonate and the role of peroxisome proliferator-activated receptor alpha.
33. Eagon, P. K, et al. s.l.: International Journal Cancer Research, 1994, Vol. 58. 736-743. Di(2-ethylhexyl)phthalate-induced changes in liver estrogen metabolism and hyperplasia.
34. Ehresman, D. G and Olsen, G. s.l.: Society of Toxicology Annual Meeting, 2005, Vol. Abstract No. 1236. 253. Evaluation of the half life (T1/2) of elimination of perfluorooctanoate (PFOA) from human serum.
35. Elcombe, C. R and Elcombe, B. M. s.l.: The Toxicologist, 2007, Vol. 46th Annual Meeting. Abstract No. 867. Characterization of the hepatomegaly induced by ammonium perfluorooctanoic acid (APFO) in rats.
36. Fan, L. Q, Cattley, R. O and Corton, J. C. s.l.: Journal of Endocrinology, 1998, Vol. 158. 237-246. Tissue specific induction of 17-beta-hydroxysteroid dehydrogenase type IV by peroxisome proliferator chemicals is dependent on the peroxisome proliferator-activated receptor alpha.
37. Fernandez Freire, P, et al. 5, s.l.: Toxicology In Vitro, 2008, Vol. 22. 1228-1233. In vitro assessment of the cytotoxic and mutagenic potential of perfluorooctanoic acid.
38. Fuji, C et al. Int J Cancer 2005 114(2):209-18. Expression of serine/threonine kinase PIM-3 in hepatocellular carcinoma development and its role in proliferation of human hepatoma cell lines.
39. Gavin, C. E, et al. s.l.: Toxicologist, 1997, Vol. 36. 1180. Species differences in expression of pancreatic cholecystokinin-A receptors.
40. Gavin, C. E, Martin, N. P and Schlosser, M. J. s.l.: Toxicologist, 1996, Vol. 30. 334. Absence of specific CCK-A binding sites on human pancreatic membranes.
41. Gibson, S. J and Johnson, J. D. s.l.: Riker Laboratories Inc., Subsidiary of 3M, 1979. Absorption of FC-143-14C in rats after a single oral dose.
42. Gilliland, F. D and Mandel, J. S. 5, s.l.: American Journal of Industrial Medicine, 1996, Vol. 29. 560-568. Serum perfluorooctanoic acid and hepatic enzymes, lipoproteins, and cholesterol: A study of occupationally exposed men.
43. Gilliland, F. D and Mandel, J. S. s.l.: Journal of Occupational & Environmental Medicine, 1993, Vol. 35. 950-954. Mortality among employees of a perfluorooctanoic acid production plant.
44. Gong, J; et al. J Surg Res 2009 153(1):17-22. Serine/threonine kinase PIM-2 promotes liver tumourigenesis induction through mediating survival and preventing apoptosis of liver cell.
45. Griffith, F. D and Long, J. E. 8, s.l.: American Industrial Hygiene Association Journal, 1980, Vol. 41. 576-583. Animal toxicology studies with ammonium perfluorooctanoate.
46. Hammerman, P S et al. Blood 2005 105(11):4477-83. PIM and Akt oncogenes are independent regulators of hematopoietic cell growth and survival.
47. Hanhijarvi, H, et al. s.l.: Pharmacology & Toxicology, 1987, Vol. 61.66-68. Elimination and toxicity of perfluorooctanoic acid during subchronic administration in the wistar rat.
48. Hanhijarvi, H, Ophaug, R. H and Singer, L. 1, s.l.: Proceedings of the Society for Experimental Biology and Medicine, 1982, Vol. 171. 50-55. The sex-related difference in perfluorooctanoate excretion in the rat.
49. Harada, K, et al. 2, s.l.: Environmental Research, 2005, Vol. 99.253-261. Renal clearance of perfluorooctane sulfonate and perfluorooctanoate in humans and their species-specific excretion.
50. Harding, H. P and Ron, D. s.l.: Diabetes, 2002, Vol. 51. S455-461. Endoplasmic reticulum stress and the development of diabetes.
51. He H C, et al. Chin Med J (Engl). 2007 Sep. 5; 120(17):1491-3. Detection of PIM-1 mRNA in prostate cancer diagnosis.
52. Healy, S. S, et al. s.l.: European Journal of Pharmacology, 2009, Vol. October 14 epub. Targeting the endoplasmic reticulum-stress response as an anticancer strategy-epub 10. 1016/j.ejphar, 2009. 06.064.
53. Hertz, R, Bisharashieban, J and Bartana, J. s.l.: Journal of Biological Chemistry, 1995, Vol. 270. 13470-13475. Mode of action of peroxisome proliferators as hypolipidemic drugs—Suppression of apolipoprotein CIII.
54. Hogan, C; et al. J Biol Chem 2008, 283(26):18012-18023. Elevated levels of oncogenic protein kinase PIM-1 induce the p53 pathway in cultured cells and correlate with increased MDM2 in mantle cell lymphoma.
55. Holzer, J, et al. 5, s.l.: International Journal of Hygiene & Environmental Health, 2009, Vol. 212. 499-504. One year follow-up of perfluorinated compounds in plasma of German residents from Arnsberg formerly exposed to PFOA-contaminated drinking water.
56. Hu, X. Z and Hu, D. C. s.l.: Archives Toxicology, 2009, Vol. May 27 epub. Effects of perfluorooctanoate and perfluorooctanesulfonate exposure on hepatoma HepG2 cells.
57. J, Berger and Moller, D. E. s.l.: Annual Review of Medicine, 2002, Vol. 53. 409-435. The mechanisms of action of PPARs.
58. Johnson, J. D, Gibson, S. J and Ober, R. E. 6, s.l.: Fundamental and Applied Toxicology, 1984, Vol. 4. 972-976. Cholestyramine-enhanced fecal elimination of C-14 in rats after administration of ammonium C-14 perfluorooctanoate or potassium C-14 perfluorooctanesulfonate.
59. Kennedy, G. L, et al. 4, s.l.: Critical Reviews in Toxicology, 2004, Vol. 34. 351-384. The toxicology of perfluorooctanoate.
60. Kennedy, G. L. s.l.: Toxicology Letters, 1987, Vol. 39. 295-300. Increase in mouse liver weight following feeding of ammonium perfluorooctanoate and related fluorochemicals.
61. Kersten, S, B, Desvergne and Wahli, W. s.l.: Nature, Vol. 405.421-424. Roles of PPARs in health and disease.
62. Kim, R, et al. s.l.: Apoptosis, 2006, Vol. 11. 5-13. Role of the unfolded protein response in cell death.
63. Kim, K T et al. Blood 2005, 105(4):1759-67. PIM-1 is upregulated by constitutively activated FLT3 and plays a role in Flt3-mediated cell survival.
64. Klaunig, J. E, et al. s.l.: Critical Reviews in Toxicology, 2003, Vol. 33. 655-780. PPAR alpha agonist induced rodent tumours: Modes of action and human relevance.
65. Kleszczynski, K and Skladanowski, A. C. 3, s.l.: Toxicology Applied Pharmacology, 2009, Toxicol. Appl. Pharmacol. 234(3), Vol. 234, pp. 300-305. 300-305. Mechanism of cytotoxic action of perfluorinated acids. I. Alteration in plasma membrane potential and intracellular pH level.
66. Koeffler, H. P. s.l.: Clinical Cancer Research, 2003, Vol. 9. 1-9. Peroxisome proliferator-activated receptor gamma and cancers.
67. Kudo, N, et al. 3, s.l.: Chemico Biological Interactions, 2002, Vol. 139. 301-316. Sex-hormone regulated renal transport of perfluorooctanoic acid.
68. Kuslikis, B. I, Vanden Heuvel, J. P and Peterson, R. E. 1, s.l.: Journal of Biochemical Toxicology, 1992, Vol. 7. 25-29. Lack of evidence for perfluorodecanoyl- or perfluorooctanoyl-coenzyme A formation in male and female rats.
69. Lee, A. H and Glimcher, L. H. s.l.: Cellular and Molecular Life Sciences, 2009, Vol. 66. 2835-2850. Intersection of the unfolded protein response and hepatic lipid metabolism.
70. Lee, A. H, et al. s.l.: Science, 2008, Vol. 320. 1492-1496. Regulation of hepatic lipogenesis by the transcription factor XBP1.
71. Li, Y Y et al. Cancer Res 2006 66(13):6741-7. PIM-3, a proto-oncogene with serine/threonine kinase activity, is aberrantly expressed in human pancreatic cancer and phosphorylates bad to block bad-mediated apoptosis in human pancreatic cancer cell lines.
72. Li Y Y, et al. Cancer Sci. 2009 March; 100(3):396-404. Epub 2008 Dec. 16. Essential contribution of Ets-1 to constitutive PIM-3 expression in human pancreatic cancer cells.
73. Lin, Y W et al. Blood 2010, 115(4):824-33. A small molecule inhibitor of PIM protein kinases blocks the growth of precursor T-cell lymphoblastic leukemia/lymphoma.
74. Liu, R. C. M, et al. s.l.: Fundamental & Applied Toxicology, 1996, Vol. 30. 102-108. Effect of the peroxisome proliferator, ammonium perfluorooctanoate (APFO), on hepatic aromatase activity in adult male Crl:CDBR (CD) rats.
75. Liu, R. C. M, Hahn, C and Hurtt, M. E. s.l.: Fundamental & Applied Toxicology, 1996, Vol. 30. 220-228. The direct effect of hepatic peroxisome proliferators on rat Leydig cell function in vitro.
76. Marciniak, S. J and Ron, D. s.l.: Physiological Reviews, 2006, Vol. 86. 1133-1149. Endoplasmic reticulum stress signalling in disease.
77. Moenner, M et al: Cancer Res, 2007, vol 67. 10631-10634. Integrated Endoplasmic Reticulum Stress Responses in Cancer.
78. Morimura, K, et al. s.l.: Carcinogenesis, 2006, Vol. 27. 1074-1080. Differential susceptibility of mice humanized for peroxisome proliferator-activated receptor to WY14,643-induced liver tumourigenesis.
79. Mumenthaler, S M et al. Mol Cancer Ther. 2009 8(10):2882-93. Pharmacologic inhibition of PIM kinases alters prostate cancer cell growth and resensitizes chemoresistant cells to taxanes.
80. Ordonez, N G. 3, s.l.: Advances in Anatomic Pathology, 2001, Vol. 8. 144-159. Pancreatic acinar cell carcinoma.
81. Nakagawa, H, et al. s.l.: Basic Clinical Pharmacology & Toxicology, 2009, Vol. April 3 epub. Human organic anion transporter hOAT4 is a transporter of perfluorooctanoic acid.
82. Nga, M E et al. Int J Exp Pathol 2010, 91(1):34-43. PIM-1 kinase expression in adipocytic neoplasms: diagnostic and biological implications.
83. Obourn, J. D, et al. s.l.: Toxicology & Applied Pharmacology, 1997, Vol. 145. 425-436. Mechanisms for the pancreatic oncogenic effects of the peroxisome proliferator Wyeth-14,643.
84. Ohmori, K, et al. s.l.: Toxicology, 2003, Vol. 184. 135-140. Comparison of the toxicokinetics between perfluorocarboxylic acids with different carbon chain length.
85. Olsen, G. W and Burris, J. M. 4, s.l.: Drug and Chemical Toxicology, 2000, Vol. 23. 603-620. Plasma cholecystokinin and hepatic enzymes, cholesterol and lipoproteins in ammonium perfluorooctanoate production workers.
86. Olsen, G. W, et al. 7, s.l.: Journal of Occupational and Environmental Medicine, 1998, Vol. 40. 614-622. An epidemiologic investigation of reproductive hormones in men with occupational exposure to perfluorooctanoic acid.
87. Olsen, G. W, et al. s.l.: Journal of Occupational & Environmental Medicine, 2003, Vol. 45. 260-270. Epidemiological assessment of worker serum perfluorooctanesulfonate (PFOA) and perfluorooctanoate (PFOA) concentrations and medical surveillance examinations.
88. Pandol, S. J. Eds: M. Sleisenger & J. S Fortran, s.l.: Gastointestinal and liver diseases, 1998, Vol. 1. 771-782. Pancreatic physiology and secretory testing.
89. Perkins, R. G and Butenhoff, J. L. 4, s.l.: Drug and Chemical Toxicology, 2004, Vol. 27.361-378. 13-week dietary toxicity study of ammonium perfluorooctanoate (APFO) in male rats.
90. Permadi, H, et al. 6, s.l.: Biochemical Pharmacology, 1992, Vol. 44. 1183-1191. Effects of perfluoro fatty acids on xenobiotic-metabolizing enzymes, enzymes which detoxify reactive forms of oxygen and lipid peroxidation in mouse liver.
91. Popivanova, B K et al. Cancer Sci 2007 98(3):321-8. Proto-oncogene, PIM-3 with serine/threonine kinase activity, is aberrantly expressed in human colon cancer cells and can prevent Bad-mediated apoptosis.
92. Reddy, J. K and Rao, M. S. s.l.: Journal of the National Cancer Institute, 1977, Vol. 59. 1645-1650. Malignant tumours in rats fed nafenopin, a hepatic peroxisome prolferator.
93. Reiser-Erkan, C et al. Cancer Biol Ther 2008, 7(9):1352-9. Hypoxia-inducible proto-oncogene PIM-1 is a prognostic marker in pancreatic ductal adenocarcinoma.
94. Ren, H, et al. 3-4, s.l.: Reproductive Toxicology, 2009, Vol. 27.266-277. Evidence for the involvement of xenobiotic-responsive nuclear receptors in transcriptional effects upon perfluoroalkyl acid exposure in diverse species.
95. Riker. Aug. 29, 1987, s.l.: USEPA Public Docket AR-226-0437, 1987, Vol. Experiment Number 0281 CR0012. Two year oral (diet) toxicity/carcinogenicity study of fluorochemical FC-143 in rats.
96. Roh, M et al. PLoS One 2008, 3(7):32572. A role for polyploidy in the tumourigenicity of PIM-1 expressing human prostate and mammary epithelial cells.
97. Rosen, M. B, et al. 1, s.l.: Toxicological Sciences, 2008, Vol. 103.46-56. Toxicogenomic dissection of the perfluorooctanoic acid transcript profile in mouse liver: Evidence for the involvement of nuclear receptors PPARalpha and CAR.
98. Sakr, C. J, et al. 10, s.l.: Journal of Occupational & Environmental Medicine, 2007, Vol. 49. 1086-1096. Cross-sectional study of lipids and liver enzymes related to serum biomarker of exposure (ammonium perfluororooctanoate or APFO) as part of a general health survey in a cohort of occupationally exposed workers.
99. Sakr, C. J, et al. s.l.: Occupational & Environmental Medicine, 2009, Vol. June 23 epub. Ischemic heart disease mortality among workers with occupational exposure to ammonium perfluorooctanoate.
100. Schroder, M. 6, s.l.: Cell & Molecular Life Sciences, 2008, Vol. 65. 862-894. Endoplasmic reticulum stress responses.
101. Shah, N et al. P Eur J Cancer 2008 44(15):2144-51. Potential roles for the PIM1 kinase in human cancer—a molecular and therapeutic appraisal.
102. Shi, Y. H, Hon, M and Evans, R. M. s.l.: Proceedings of the National Academy of Sciences of the USA, 2002, Vol. 99. 2613-2618. The peroxisome proliferator-activated receptor delta, an integrator of transcriptional repression and nuclear receptor signaling.
103. Staels, B and Auwerx, J. s.l.: Current Pharmaceutical Design, 1997, Vol. 3. 1-14. Role of PPAR in the pharmacological regulation of lipoprotein metabolism by fibrates and thiazolidinediones.
104. Strasser, A and Puthalakath, H: Cell Death and Differentiation, 2008, vol 15. 223-225. Fold up or perish: unfolded protein response and chemotherapy.
105. Szegezdi, E, et al. 9, s.l.: EMBO Journal, 2006, Vol. 7.880-885. Mediators of endoplasmic reticulum stress-induced apoptosis.
106. Takacs, M. L and Abbott, B. D. 1, s.l.: Toxicological Sciences, 2007, Vol. 95. 108-117. Activation of mouse and human peroxisome proliferator-activated receptors (alpha, beta/delta, gamma) by perfluorooctanoic acid and perfluorooctance sulfonate.
107. Ubel, F. A, Sorenson, S. D and E. E, Roach. 8, s.l.: American Industrial Hygiene Association Journal, 1980, Vol. 41. 584-589. Health-status of plant workers exposed to fluorochemicals—a preliminary report.
108. Upham, B. L, et al. 4, s.l.: Environmental Health Perspectives, 2009, Vol. 117. 545-551. Structure-activity-dependent regulation of cell communication by perfluorinated fatty acids using in vivo and in vitro model systems.
109. USEPA. s.l.: Office of Pollution Prevention and Toxics Risk Assessment Division, 2005. US Environmental Protection Agnecy, Draft Hazard Assessment of Perfluorooctanoic acid and its salts.
110. Vanden Heuval, J. P, et al. 2, s.l.: Journal of Biochemical Toxicology, 1991, Vol. 6. 83-92. Tissue distribution, metabolism, and elimination of perfluorooctanoic acid in male and female rats.
111. Vanden Heuvel, J. P, et al. s.l.: Toxicological Sciences, 2006, Vol. 92. 476-486. Differential activation of nuclear receptors by perfluorinated fatty acid analogs and natural fatty acids: A comparison of human, mouse, and rat peroxisome proliferator-activated receptor-alpha, -beta, and -gamma, LXR-beta, and RXR-alpha.
112. Vazquez, M, Silvestre, J. S and Prous, J. R. s.l.: Methods and Findings in Experimental and Clinical Pharmacology, 2002, Vol. 24. 515-523. Experimental approaches to study PPAR gamma agonists as antidiabteic drugs.
113. Vosper, H, et al. 47, s.l.: The Journal of Biological Chemistry, 2001, Vol. 276. 44258-44265. The peroxisome proliferator-activated receptor delta promotes lipid accumulation in human macrophages.
114. Vu-Dac, N, et al. s.l.: Journal of Clinical Investigation, 1995, Vol. 96. 741-750. Fibrates increase human apolipoprotein AIII expression through activation of the peroxisome prolferation-activated receptor.
115. Warnecke-Eberz U, et al. Anticancer Res. 2009 November; 29(11):4451-5. Prognostic impact of protein overexpression of the proto-oncogene PIM-1 in gastric cancer.
116. Weber, S. M, et al. s.l.: American Journal of Physiology, Endocrinology & Metabolism, 2004, Vol. 287. E1171-E1177. PPARgamma ligands induce ER stress in pancreatic beta-cells: ER stress activation results in attenuation of cytokine signaling.
117. Wolf, C et al: Toxicological Sciences, 2008, vol 106.162-171. Activation of Mouse and Human Peroxisome Proliferator—Activated Receptor Alpha by Perfluoralkyl Acids of Different Functional Groups and Chain Lengths.
118. Wu, L. L, et al. 31, s.l.: BMC Structural Biology, 2009, Vol. 9.1-7. Interaction of perfluorooctanoic acid with human serum albumin.
119. Wu, Y et al. Oncogene 2010, Jan. 18 epub. Accelerated heaptocellular carcinoma development in mice expressing the PIM-3 transgene selectively in the liver.
120. Xu Y, et al. J Surg Oncol. 2005 Dec. 15; 92(4):326-30. Overexpression of PIM-1 is a potential biomarker in prostate carcinoma.
121. Yang, C, et al. 3-4, s.l.: Reproductive Toxicology, 2009, Vol. 27. 299-306. Differential effects of peripubertal exposure to perfluorooctanoic acid on mammary gland development in C57/BI/6 and Balb/c mouse strains.
122. Yang, Q and Xie, Y: Depierre, J. W. 2, s.l.: Clinical and Experimental Immunology, 2000, Vol. 122. 219-226. Effects of peroxisome proliferators on the thymus and spleen of mice.
123. Yang, Q, et al. s.l.: Biochemical Pharmacology, 2001, Vol. 62. 1133-1140. Further evidence for the involvement of inhibition of cell proliferation and development in thymic and splenic atrophy induced by the peroxisome proliferator PFOA in mice.
124. Yang, Q, et al. s.l.: Biochemical Pharmacology, 2002, Vol. 63. 1893-1900. Involvement of the peroxisome proliferator-activated receptor alpha in the immunomodulation caused by peroxisome proliferators in mice.
125. Yang, Q, et al. s.l.: International Immunopharmacology, 2002, Vol. 2. 389-397. Potent suppression of the adaptive immune response in mice upon dietary exposure to the potent peroxisome proliferator, perfluorooctanoic acid.
126. Zang, C, et al. 8, s.l.: Molecular Cancer Therapy, 2009, Vol. 8, 2296-2307. Induction of endoplasmic reticulum stress response by TZD18, a novel dual ligand for peroxisome proliferator-activated receptor alpha/gamma, in human breast cancer cells.
127. Zhang, K: Kaufman, R. J. s.l.: Nature, 2008, Vol. 454. 455-462. From endoplasmic-reticulum stress to the inflammatory response.
128. Zheng, H C et al. J Cancer Res Clin Oncol 2008 134(4):481-8. Aberrant PIM-3 expression is involved in gastric adenoma-adenocarcinoma sequence and cancer progression.
129. Zheng H C, et al. J Cancer Res Clin Oncol. 2008, 134(4):481-8. Epub 2007 Sep. 18. Aberrant PIM-3 expression is involved in gastric adenoma-adenocarcinoma sequence and cancer progression.

1. A composition comprising between 10 mg and 2000 mg of an active ingredient per dosage unit, wherein the active ingredient is perfluorooctanoic acid (PFOA) or a salt, derivative or variant thereof.

2. The composition of claim 1, wherein the PFOA is ammonium perfluorooctanoic acid (APFO).

3.-6. (canceled)

7. The composition of claim 1, wherein the composition further comprises a pharmaceutically acceptable excipient, diluent, carrier or filler.

8.-10. (canceled)

11. A method of treating cancer comprising administering to a patient in need thereof an effective amount of a composition as defined in claim 1, in a single dosage at a frequency of twice per week or less.

12. The method of claim 11, wherein the effective amount is between 1 mg/kg and 7 mg/kg

13. (canceled)

14. The method of claim 11, wherein the single dosage is administered at a frequency of once per six weeks or less.

15. The method of claim 11, wherein the single dosage is between 50 mg and 1200 mg and is administered at a frequency of once per week or less.

16.-19. (canceled)

20. The method of claim 11, wherein the cancer pancreatic cancer, ovarian cancer, breast cancer, prostate cancer, liver cancer, chondrosarcoma, lung cancer, head and neck cancer, colon cancer, sarcoma, leukaemia, lymphoma, kidney cancer, thyroid cancer or brain cancers, including glioblastoma.

21. (canceled)

22. The composition of claim 1, further comprising a further chemotherapeutic agent.

23. The composition of claim 22, wherein the further chemotherapeutic is selected from Doxorubicin, Gemcitabine, Roscovitine, Rapamycin, 5-FU, PARP inhibitors, kinase inhibitors, including PIM kinase inhibitors and MAP kinase inhibitors, Hsp90 inhibitors, including Geldanamycin, proteasome inhibitors, including Bortezomib, HDAC inhibitors or prodrugs thereof.

24. The composition of claim 22, wherein the further chemotherapeutic is present in an individually effective dose.

25. The composition of claim 22, wherein the further chemotherapeutic is present in a lower than individually effective dose.

26.-29. (canceled)

30. A therapeutic system for the treatment of cancer comprising a combination of components which are (i) a composition as defined in claim 1; and (ii) a further chemotherapeutic agent, wherein components (i) and (ii) are provided for the use in the treatment of cancer and components (i) and (ii) are administered in combination with one another.

31. The therapeutic system of claim 30, wherein administration of component (i) precedes administration of component (ii).

32. The therapeutic system of claim 30, wherein administration of component (ii) precedes administration of component (i).

33. The therapeutic system of claim 30, wherein administration of component (i) occurs at the same time as administration of component (ii).

34. The therapeutic system of claim 30, wherein the further chemotherapeutic is Doxorubicin, Gemcitabine, Geldanamycin, Roscovitine, Rapamycin, 5-FU, PARP inhibitors, kinase inhibitors, including MAP kinase inhibitors, or prodrugs thereof, and wherein the cancer is selected from pancreatic cancer, ovarian cancer, breast cancer, prostate cancer, liver cancer, chondrosarcoma, lung cancer, head and neck cancer, colon cancer, sarcoma, leukaemia, lymphoma, kidney cancer, thyroid cancer and brain cancers such as glioblastoma.

35.-41. (canceled)

42. A kit of parts comprising:
(i) a composition as defined in claim 1; and
(ii) a further chemotherapeutic agent.

43. The kit of claim 42 further comprising:
(iii) means of administering (i) and (ii) to a patient, wherein the administration may be at the same time or in succession.

44. The kit of claim 42, wherein the further chemotherapeutic agent is Doxorubicin, Gemcitabine, Roscovitine, Rapamycin, 5-FU, PARP inhibitors, kinase inhibitors, including PIM kinase inhibitors and MAP kinase inhibitors, Hsp90 inbhibitors, including Geldanamycin, proteasome inhibitors, including Bortezomib, HDAC inhibitors or prodrugs thereof.

45.-48. (canceled)
